(19) United States
(12) Patent Application Publication
(10) Pub. No.: US 2026/0035369 A1
(43) Pub. Date: Feb. 5, 2026

(54) VINCAMINE DERIVATIVES, PREPARATION METHOD THEREFOR, AND USE THEREOF

(71) Applicant: SOUTHWEST UNIVERSITY, Chongqing (CN)

(72) Inventors: Chong LI, Chongqing (CN); Xufei BIAN, Chongqing (CN); Yang YU, Chongqing (CN)

(21) Appl. No.: 19/353,706

(22) Filed: Oct. 9, 2025

Related U.S. Application Data
(63) Continuation of application No. PCT/CN2023/113685, filed on Aug. 18, 2023.

(30) Foreign Application Priority Data
Jun. 8, 2023 (CN) 202310676275.6

(51) Int. Cl. C07D 471/22; A61K 9/51; C07D 491/22

(52) U.S. Cl. CPC C07D 471/22; A61K 9/5123; A61K 9/5153; C07D 491/22

(57) ABSTRACT

The present application relates to a vincamine derivative, a preparation method therefor, and use thereof, and belongs to the technical field of preparation of vincamine derivatives. The vincamine derivative has the following advantages: (i) Modification of the tail chain increases the lipid solubility of vincamine compounds without affecting the cerebral blood flow regulation of vincamine itself, thereby helping the carried drug penetrate the blood-brain barrier, exert a brain-protective effect, and improve cerebral microcirculatory disorders. (ii) The tertiary amine group in the parent nucleus structure of the vincamine derivative is ionizable under acidic conditions, which enables efficient delivery and lysosomal escape of nucleic acid drugs through charge adsorption, thereby improving intracellular transport. (iii) The vincamine derivative inherits various pharmacological activities inherent to vincamine, and has high safety. Therefore, the vincamine derivative has good application prospects in brain-targeted delivery of drugs for treating brain diseases.

### CROSS-REFERENCE TO RELATED APPLICATIONS

This application is a continuation of International Application No. PCT/CN2023/113685, filed on Aug. 18, 2023, which claims priority to Chinese Patent Application No. 202310676275.6, filed on Jun. 8, 2023, both of which are hereby incorporated by reference in their entireties.

### TECHNICAL FIELD

The present application belongs to the technical field of preparation of vincamine derivatives, and relates to a vincamine derivative, a preparation method therefor, and use thereof.

### BACKGROUND

The treatment of central nervous system diseases, such as neurodegenerative diseases, brain tumors, brain infections, and stroke, is severely hampered by the blood-brain barrier, which prevents most small molecule drugs and macromolecules (such as polypeptides, nucleic acid drugs, and protein drugs) from entering the brain. Considerable research has been conducted to improve the efficiency of drug delivery into the brain, including direct administration into the central nervous system, disruption of the blood-brain barrier, and receptor-mediated delivery methods. However, direct administration into the central nervous system is invasive and may lead to infection and tissue damage. It is also limited by the diffusion distance and the rapid efflux of drugs from the central nervous system within hours. Techniques such as osmotic disruption, biochemical disruption, and ultrasound-mediated disruption of the blood-brain barrier may effectively introduce drugs into the brain; however, these transient blood-brain barrier openings also allow leakage of plasma proteins into the brain, leading to neurotoxicity, vasculopathy, and chronic neuropathological changes in the brain. Therefore, methods for the safe and effective delivery of substances that cannot penetrate the blood-brain barrier, (especially gene and nucleic acid therapies) to the central nervous system via intravenous injection still require improvement. Currently, lipid nanoparticles (LNPs) primarily composed of ionizable lipids are predominantly used to encapsulate nucleic acid drugs. However, due to their high positive charge, these lipid nanoparticles are primarily distributed to the liver upon administration. Although an LNP platform technology called SORT (Selective Organ Targeting) achieves non-hepatic delivery of nucleic acid drugs to the spleen and lungs by modulating the surface charges of the formulations, the challenge of delivering nucleic acid drugs to the brain remains unsolved.

In addition, due to steric hindrance considerations during synthesis and preparation, most ionizable lipid molecules are typically designed with simple linear tertiary amine groups to achieve pH sensitivity and endosomal escape, rather than employing cyclic tertiary amine headgroups. Only a few reports have indicated that a single cyclic tertiary amine headgroup can also interact effectively with nucleic acids; for example, the piperazine-containing lipid C12-200 can simultaneously deliver five liver-targeted siRNAs into cells. Moreover, while synthetic lipids promote RNA encapsulation and cellular uptake, they do not always have ideal biological activity. Indeed, potential cytotoxicity and immunogenicity are major drawbacks associated with cationic lipid materials, as they can activate innate immunity via the complement system and Toll-like receptors, and may also induce the production of pro-inflammatory cytokines, reactive oxygen species (ROS), and the like, particularly when long-term repeated administration is necessary. Therefore, in clinical applications of LNPs, pre-administration of glucocorticoids and antihistamines is often required.

Natural product libraries provide a rich source of compounds and play a key role in drug discovery. Small molecule natural products, in particular, often exhibit great scaffold diversity and contain various pharmacophores, making them valuable lead compounds that have significantly contributed to drug development. In recent years, some small molecule natural products have also facilitated advances in drug delivery strategies. Alkaloids are a class of nitrogen-containing alkaline organic compounds with significant biological activity. As one of the earliest classes of natural products, numerous alkaloid-based drugs have been discovered and approved by the US Food and Drug Administration (FDA). Representative drugs among indole alkaloids include vinpocetine and vincamine, which belong to the vincamine alkaloid class. These compounds exhibit a variety of pharmacological effects beneficial to the brain, cardiovascular system, blood circulation and other systems. For example, they promote the uptake and utilization of glucose and oxygen in the brain, increase ATP levels, and reduce lactic acid generation during ischemia and hypoxia; prevent excitotoxic death of brain cells; alleviate hypoxic brain injury and protect neurons; enhance the functions of dopaminergic, serotonergic and norepinephrinergic neurons; prevent ischemic damage to the brain, liver, muscle tissue and other organs; scavenge free radicals and inhibit lipid peroxidation; alleviate aging-related brain dysfunction; and improve blood lipoproteins profiles. The significant anti-inflammatory and antioxidant effects of these alkaloids are anticipated to mitigate the potential toxicity associated with conventional cationic/ionizable lipid materials. Moreover, the indole heterocycles in the vincamine alkaloids contain protonatable amine groups, which are expected to enable electrostatic with nucleic acid drugs, facilitating their encapsulation and delivery into cells. However, due to the short half-life and low bioavailability of drugs such as vinpocetine, frequent administration is required for the long-term treatment of chronic diseases such as cerebrovascular disease. Studies have shown that targeted delivery systems can be employed to prolong the circulation time of vinpocetine in the body and enhance its penetration across the blood-brain barrier, thereby allowing more drug to reach the target site. Based on the pentacyclic fused skeleton of vinpocetine, intermediates and final products from its synthesis were selected for structural modification and fatty chain derivatization. It was found that some modified vincamine derivative lipidosomes exhibit brain-targeting properties.

Therefore, vincamine-derived lipids can serve as ionizable lipids for constructing safe and efficient brain-targeted delivery system for nucleic acid drugs.

### SUMMARY

In view of this, a first objective of the present application is to provide a vincamine derivative. A second objective is to provide a preparation method therefor. A third objective is to provide use of the vincamine derivative in brain-targeted delivery of drugs for treating brain diseases.

To achieve the foregoing objectives, the present application provides the following technical solutions.

1. A vincamine derivative, having a structure represented by any one of the following formulas

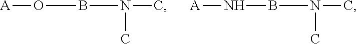

### or A-O—NH—C,

wherein A is a group derived from vincamine or a derivative thereof having an indole-fused ring system by removal of a hydroxyl group;
B is selected from the group consisting of —(CH₂)ₙ—, —(CH₂)ₙ—S—S—(CH₂)ₙ—, —(CH₂)ₙ-TK-(CH₂)ₙ— or —(CH₂)ₙ—S-Mal-(PEG)ₘ-NHS—, wherein n and m are independently positive integers; and
C is selected from the group consisting of a linear alkyl group having 10 to 18 carbon atoms, a linear alkenyl group having 10 to 18 carbon atoms, a hydroxyl-substituted linear alkyl group having 10 to 18 carbon atoms, a group of formula CₓH₂ₓ₊₁OCO(CH₂)_{y}—, a group of formula (CₐH₂ₐ₊₁)₂N(CH₂)_{b}—, a group of formula (C_{d}H_{2d+1}OCH₂CH₂)₂NCO(CH₂)ₑ—, a group of formula (C_{d}H_{2d+1}COOCH₂CH₂)₂NCO(CH₂)ₑ—, DSPE-PEG-, PEG-, a group derived from doxorubicin by removal of an amino group, a group derived from paclitaxel by carboxylation at the 2′-OH site, and an amino acid chain, wherein x, a, b, d, and e are positive integers.

Preferably, A is selected from structural formulas A1 to A12:

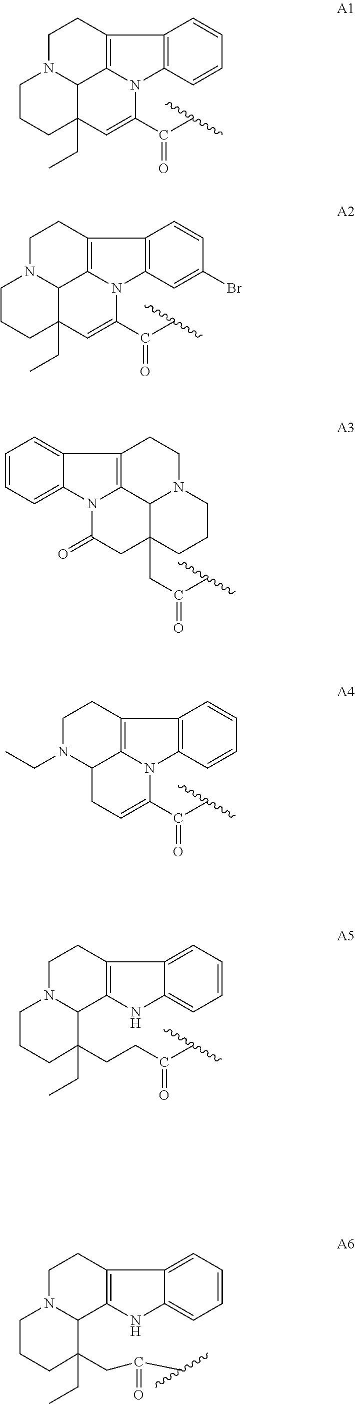 [SMILES: *C(=O)C1=CC2(CC)CCCN3CCc4c(n1c1ccccc41)C32 ; *C(=O)C1=CC2(CC)CCCN3CCc4c(n1c1cc(Br)ccc41)C32 ; *C(=O)CC12CCCN3CCc4c(n(c5ccccc45)C(=O)C1)C32 ; *C(=O)C1=CCC2c3c(c4ccccc4n31)CCN2CC ; *C(=O)CCC1(CC)CCCN2CCc3c([nH]c4ccccc34)C21 ; *C(=O)CC1(CC)CCCN2CCc3c([nH]c4ccccc34)C21]

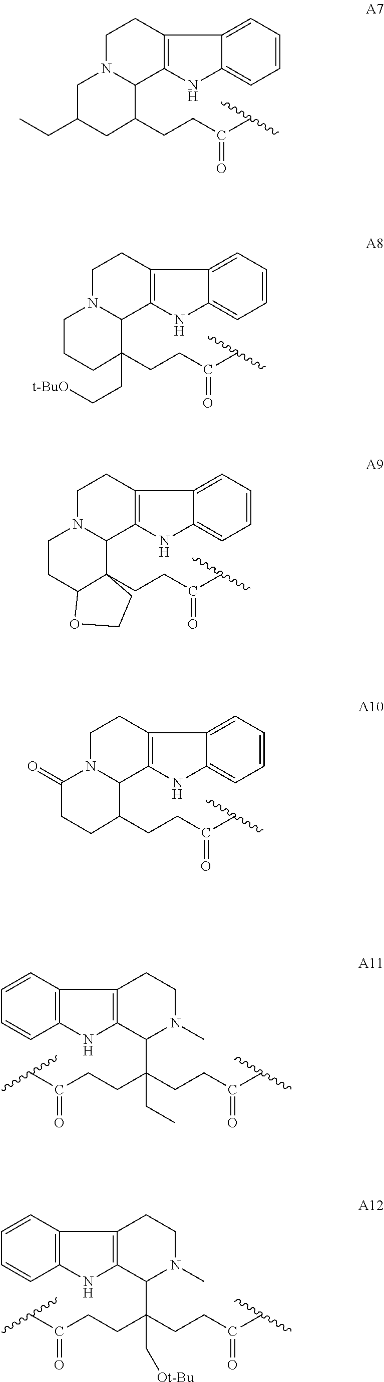 [SMILES: *C(=O)CCC1CC(CC)CN2CCc3c([nH]c4ccccc34)C12 ; *C(=O)CCC1(CCOC(C)(C)C)CCCN2CCc3c([nH]c4ccccc34)C21 ; *C(=O)CCC12CCOC1CCN1CCc3c([nH]c4ccccc34)C12 ; *C(=O)CCC1CCC(=O)N2CCc3c([nH]c4ccccc34)C12 ; *C(=O)CCC(CC)(CCC(*)=O)C1c2[nH]c3ccccc3c2CCN1C ; *C(=O)CCC(CCC(*)=O)(COC(C)(C)C)C1c2[nH]c3ccccc3c2CCN1C]

Preferably, B is selected from B1 to B5, wherein B1 is —(CH₂)₂—, B2 is —(CH₂)₆—, B3 is —(CH₂)₂—S—S—(CH₂)₂—, B4 is —(CH₂)₂—S—C(CH₃)₂—S—(CH₂)₂—, and B5 is —(CH₂)₂—S-Mal-PEG-NHS—.

Preferably, C or C′ is selected from structural formulas C1 to C14:

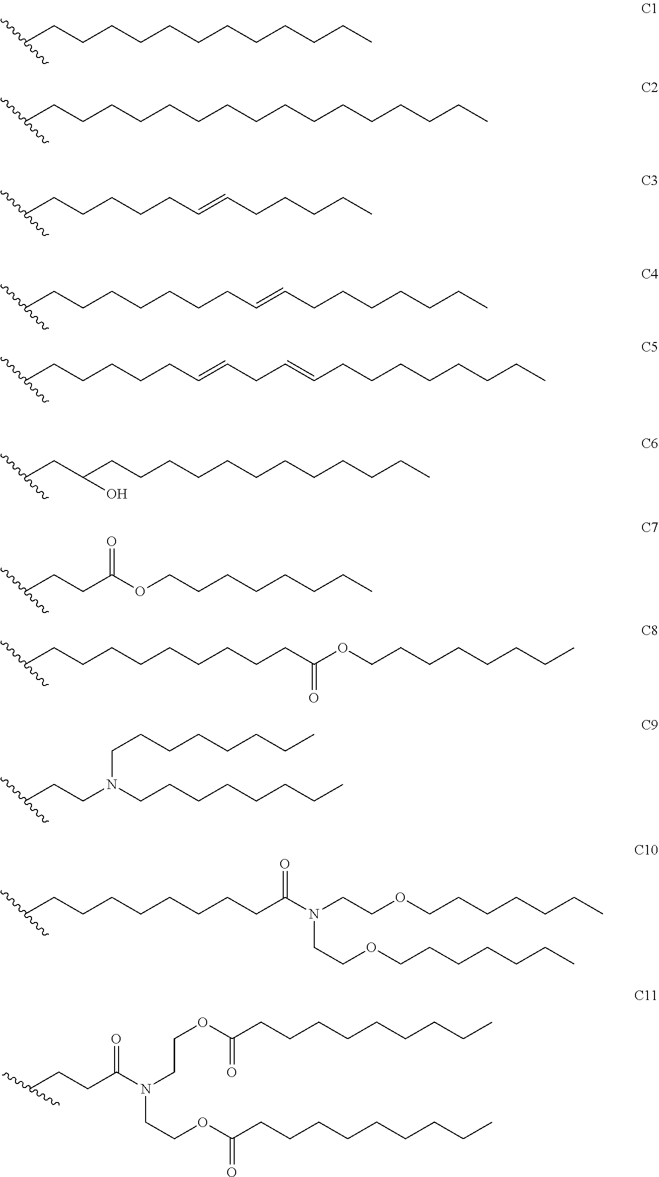

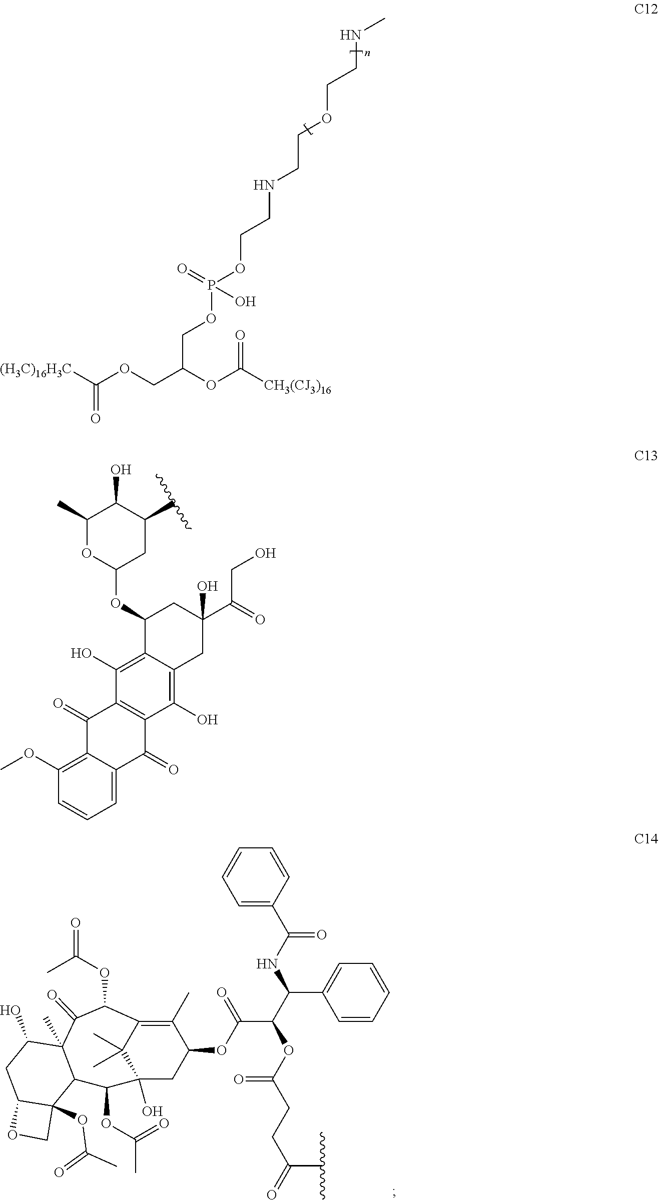

C also includes C15, wherein C15 is bovine serum albumin (BSA) having the amino acid sequence of SEQ ID NO: 1.

Preferably, the vincamine derivative is selected from compound 1 to 38, having the following structures:

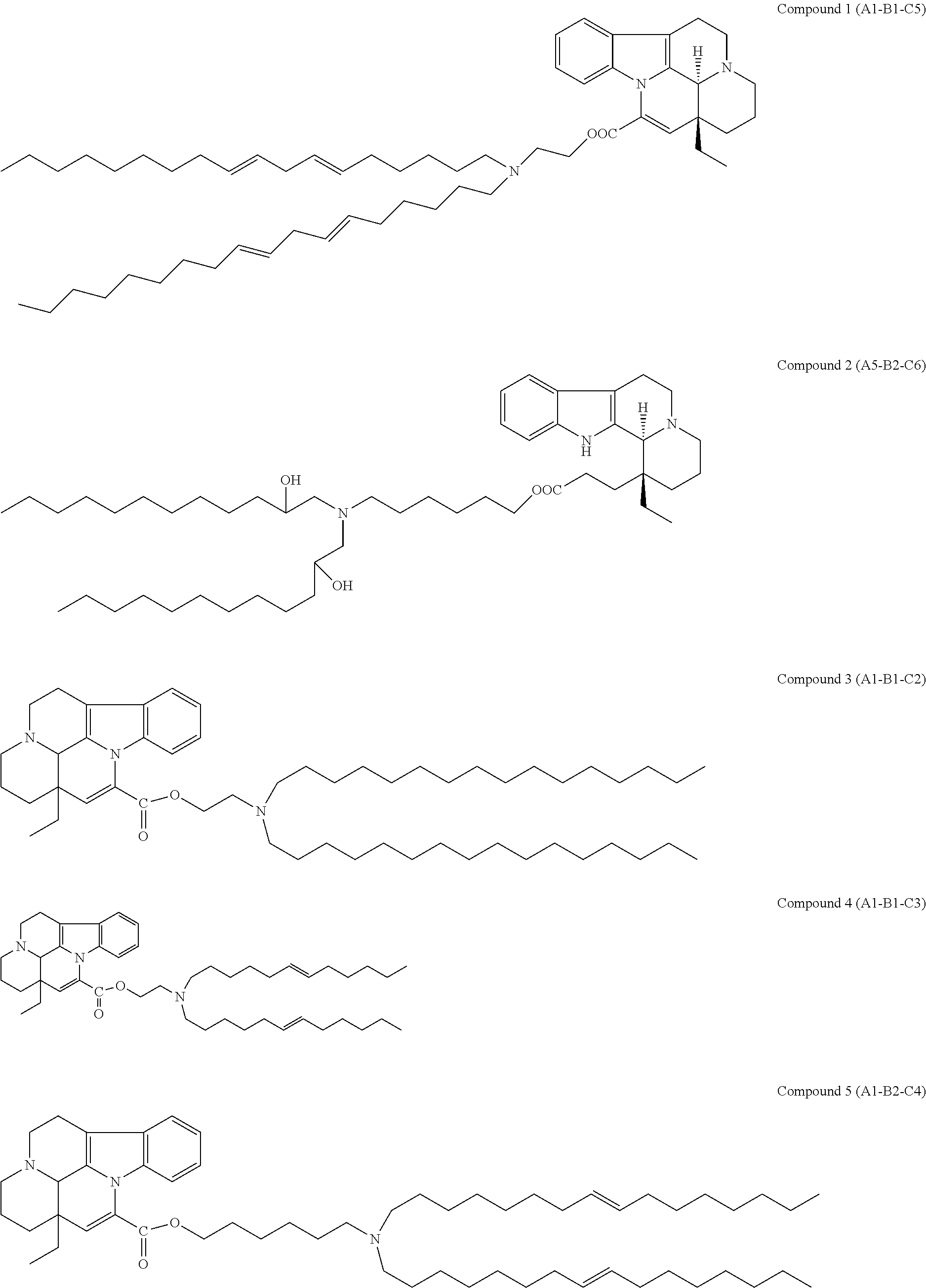

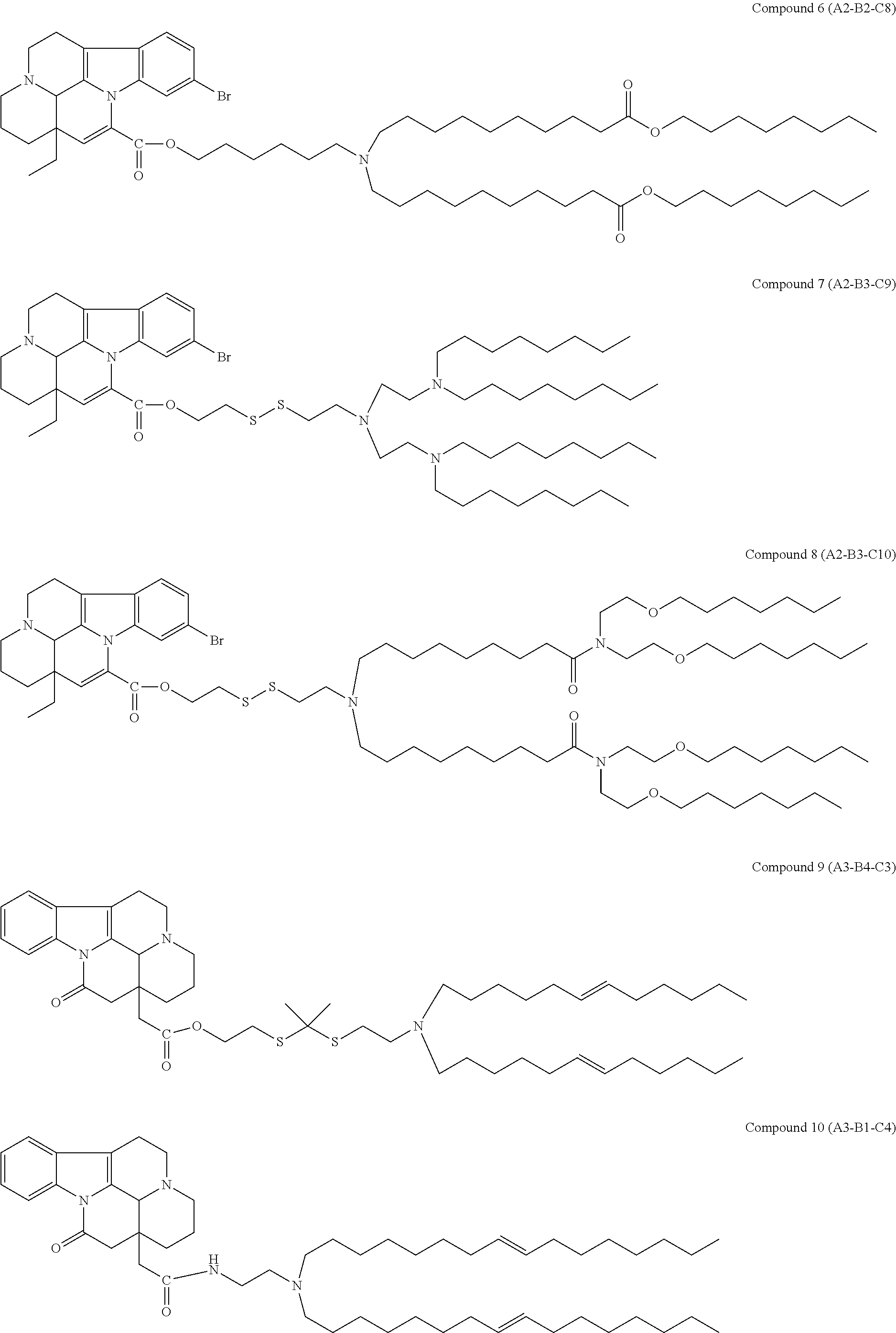

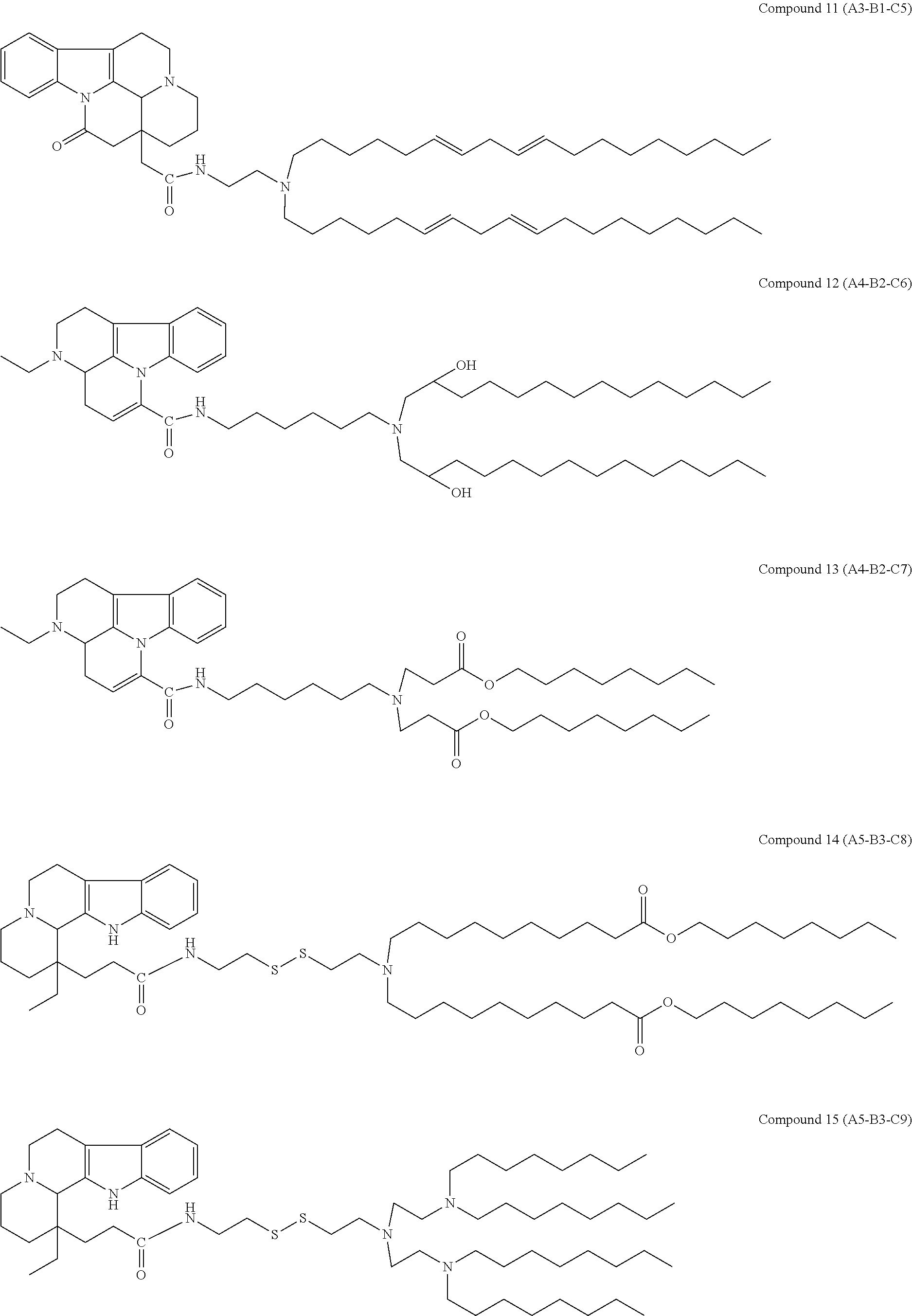

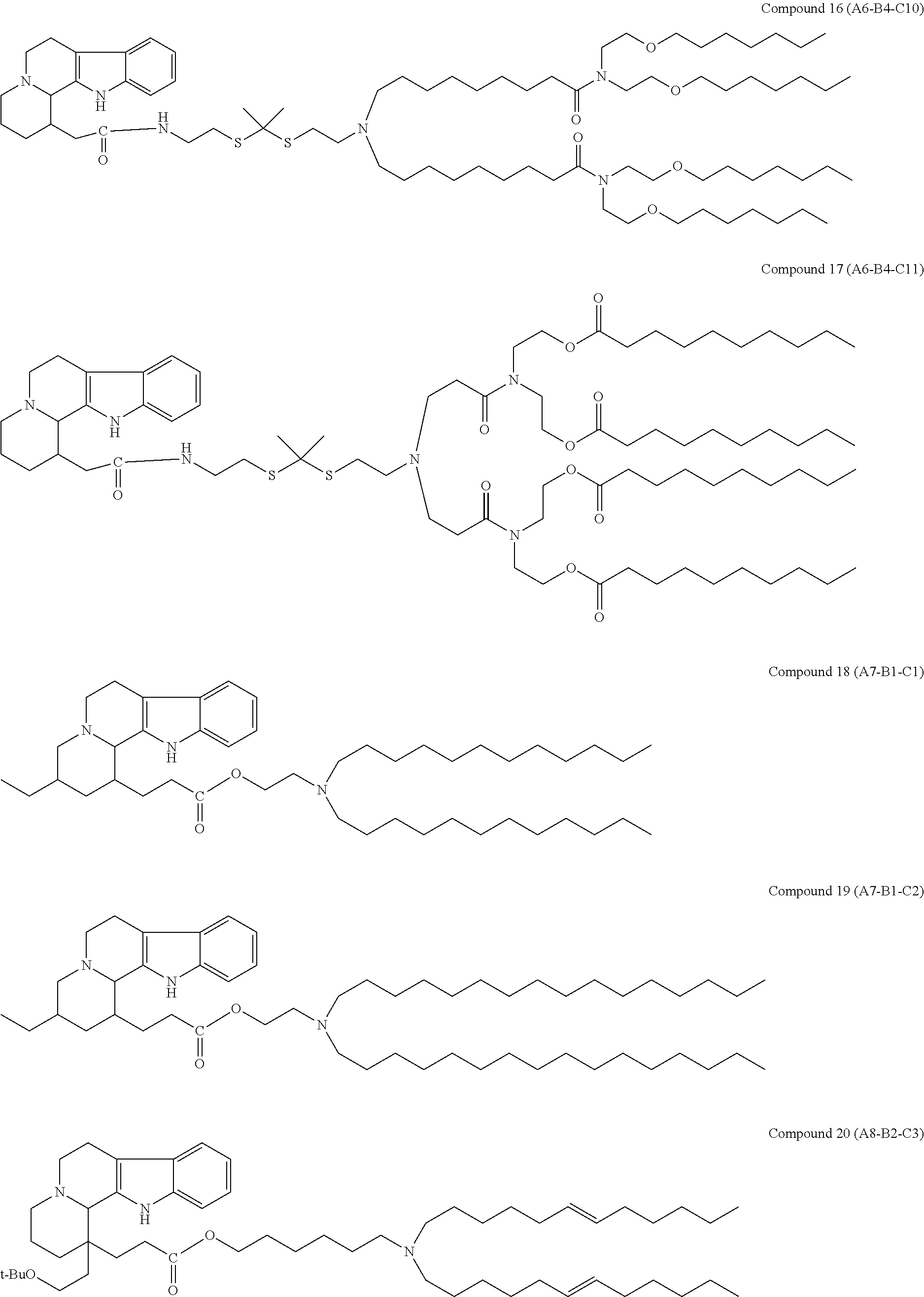

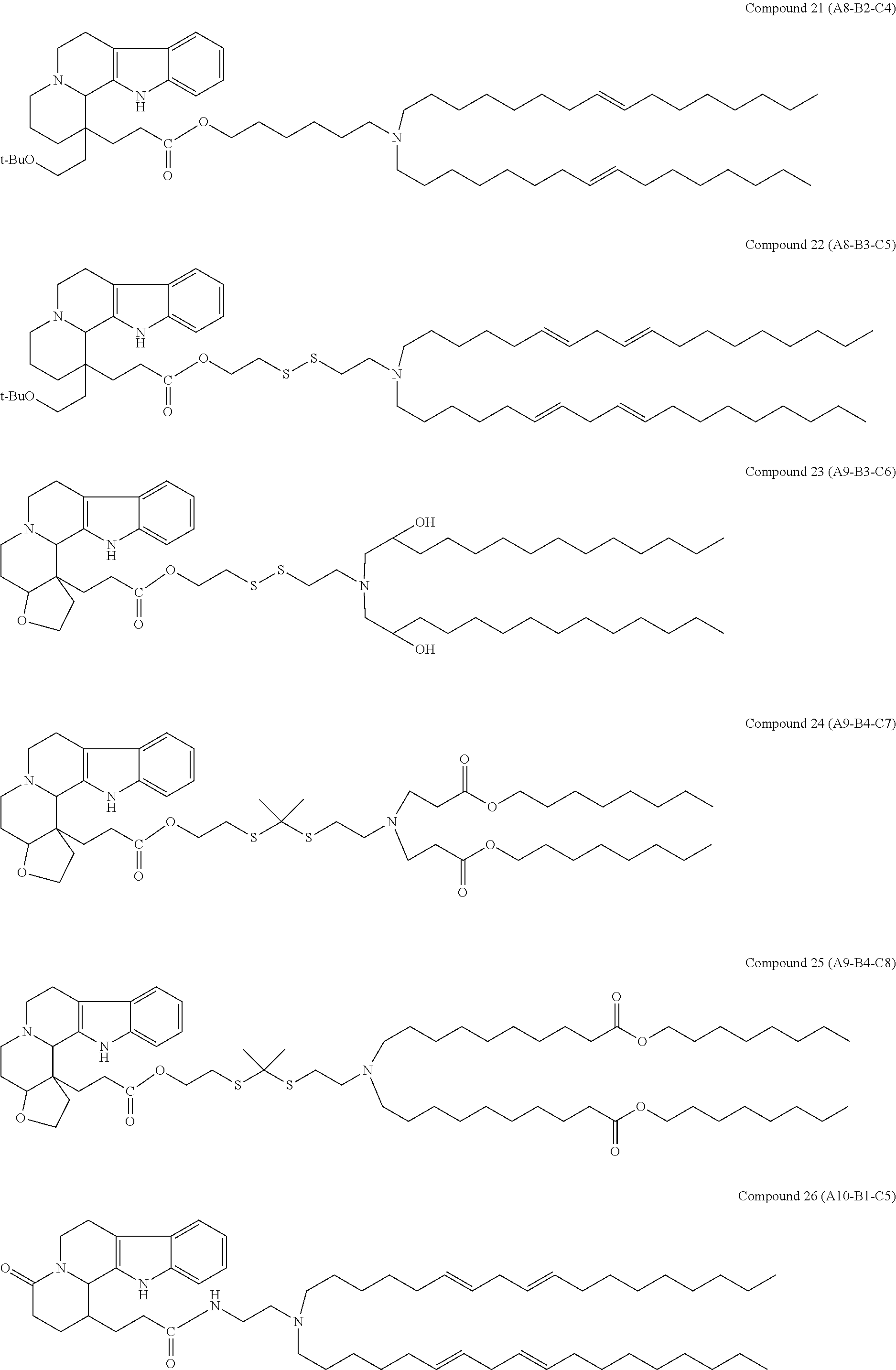

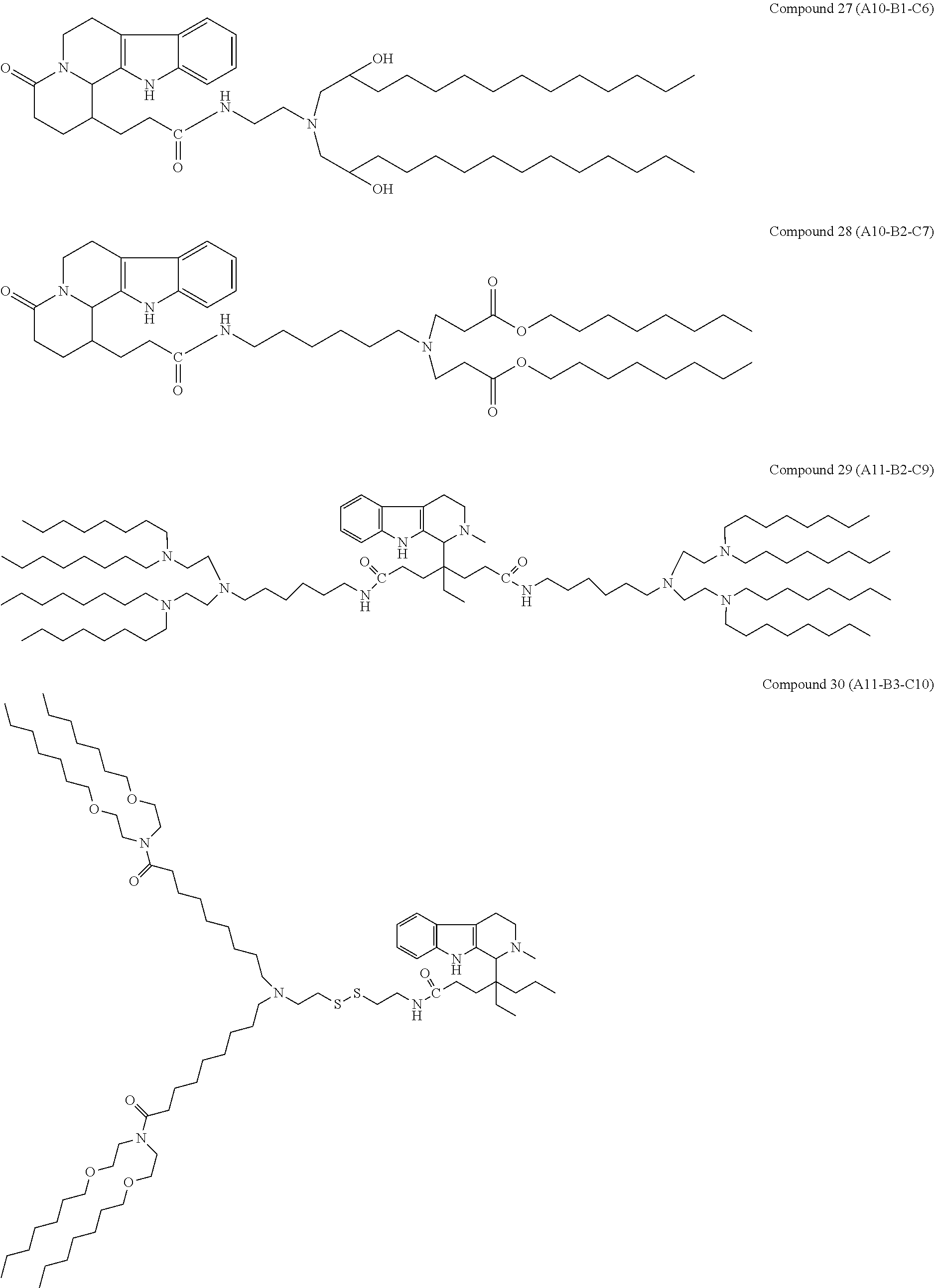

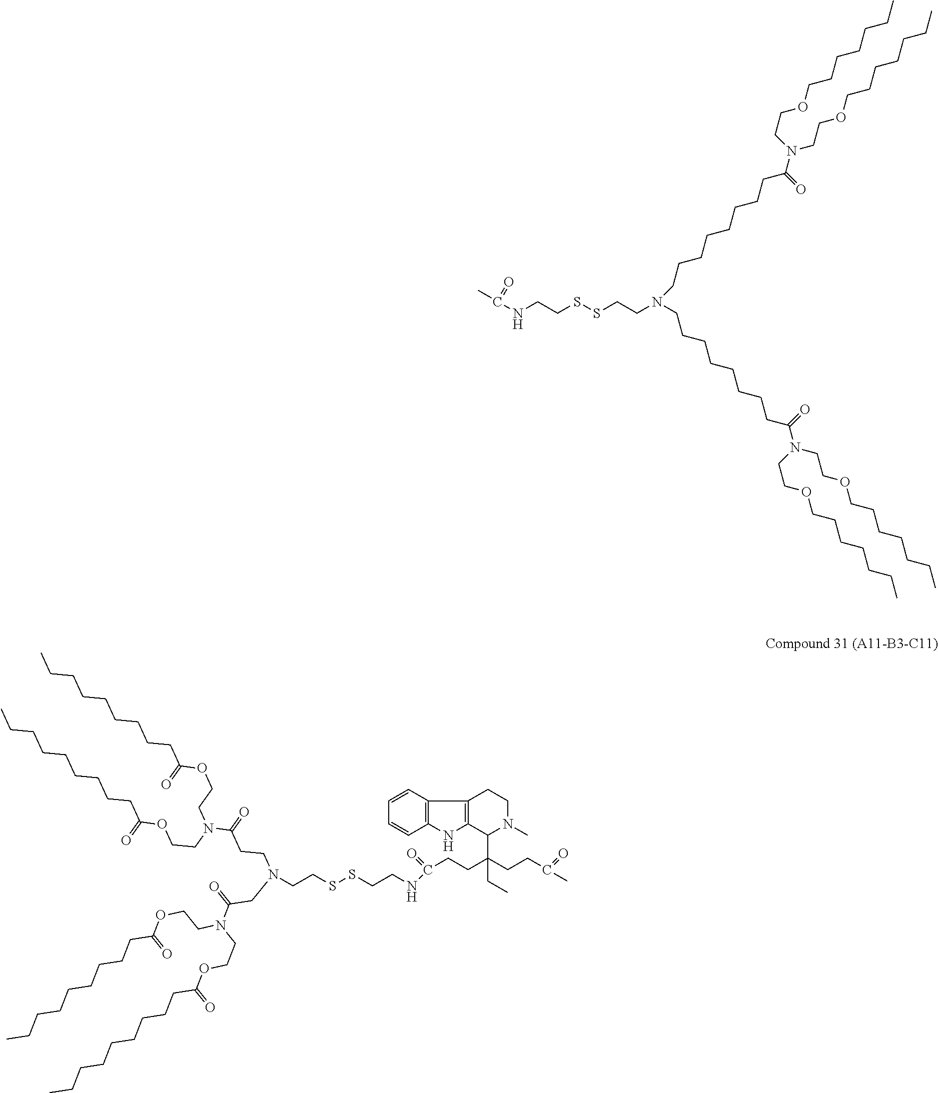

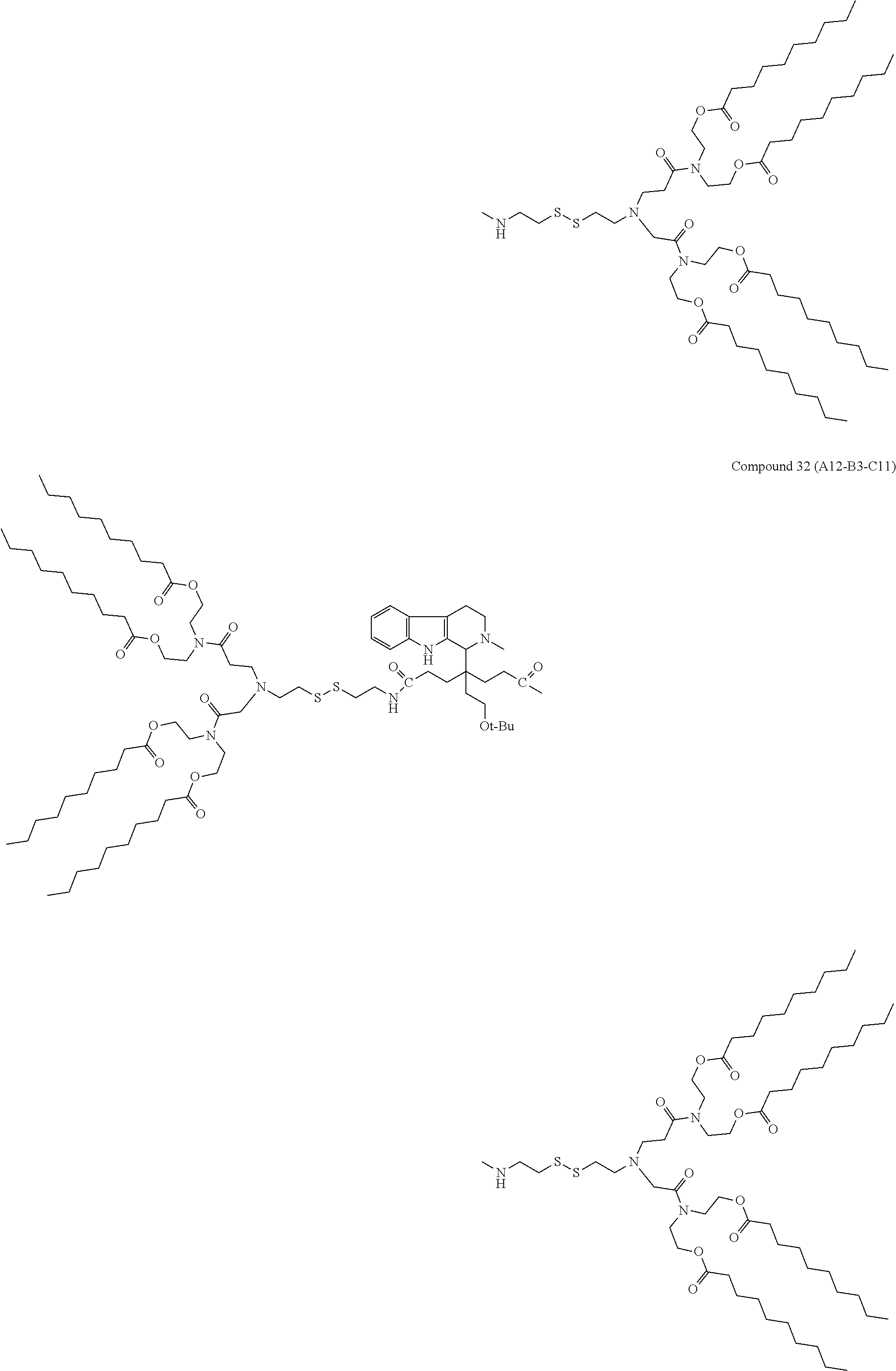

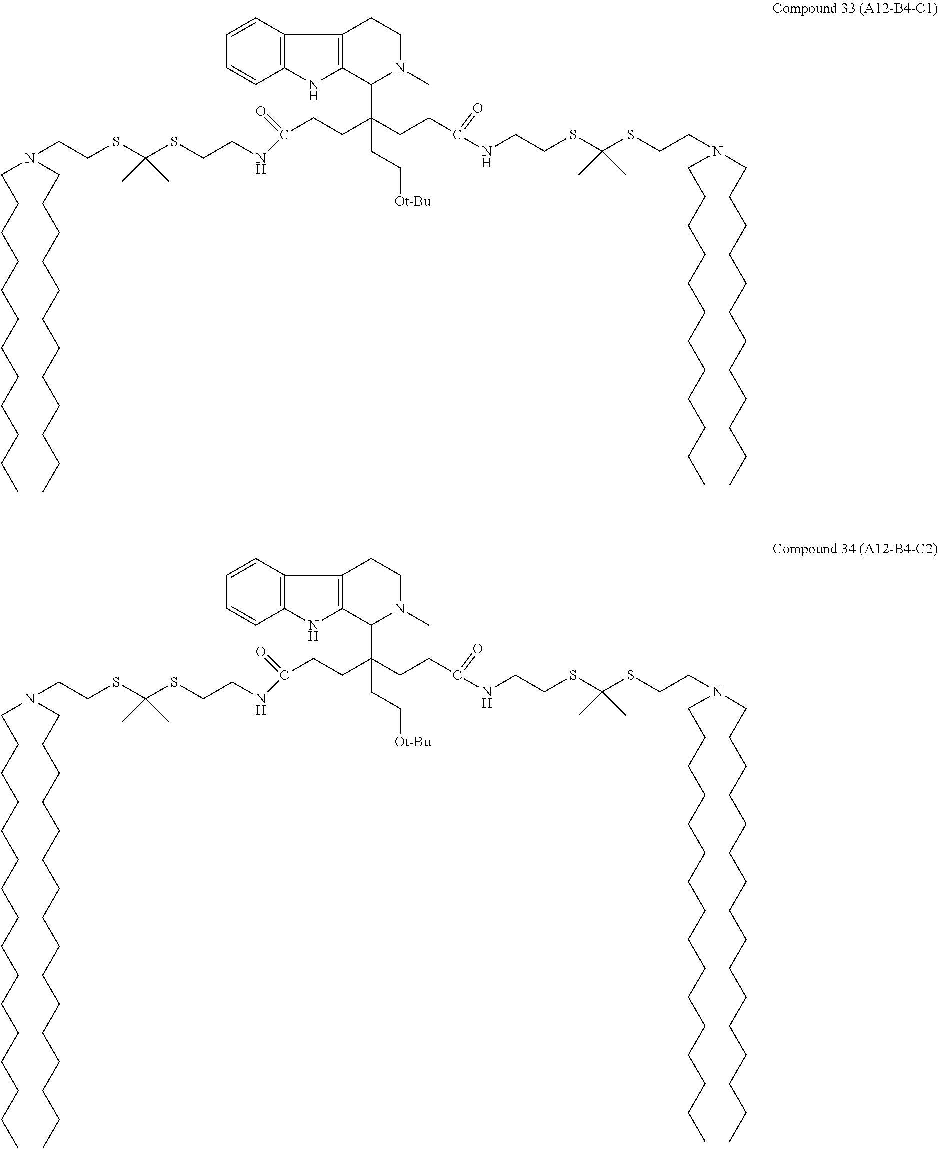

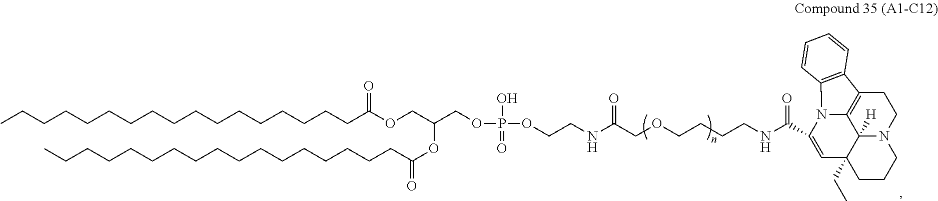

wherein n is a positive integer

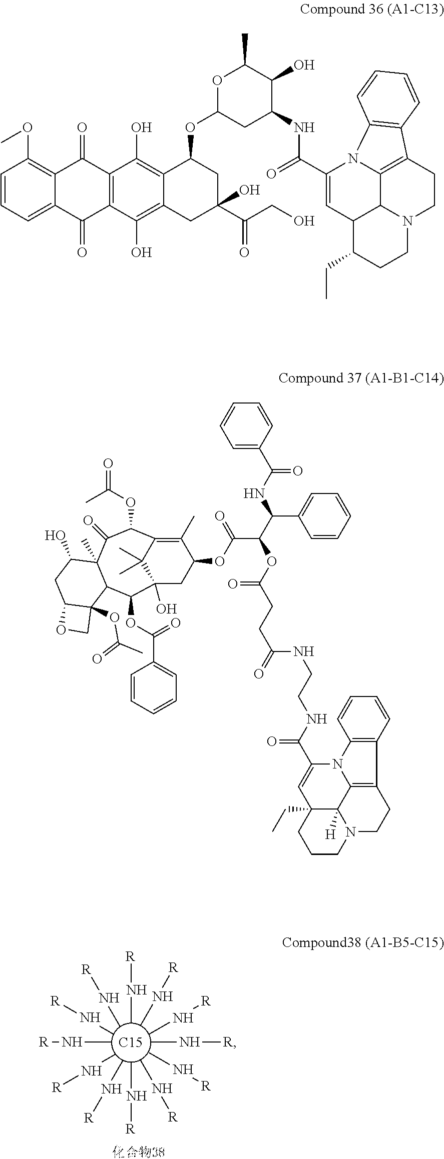

wherein the C15 is bovine serum albumin, R is

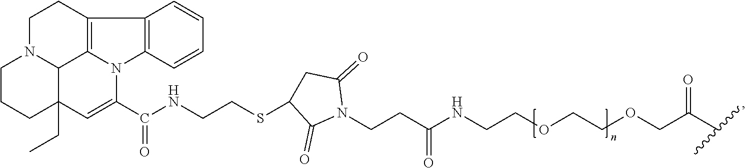

and n is a positive integer.

2. A method for preparing the vincamine derivative, wherein the method includes any one of Method I, Method II, Method III, and Method IV, when the vincamine derivative has a structure represented by formula

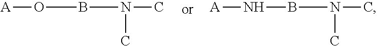

Method I includes: when A is a group with the structural formula A1, B is a group with the structural formula B1, and C is a group with the structural formula C14, dissolving a compound having the structural formula A1-OH, a compound having the structural formula H₂N-B1-NH₂, and benzotriazol-1-yl-oxy-tris(pyrrolidino)phosphonium hexafluorophosphate (PyBOP) in an organic solvent in a molar ratio of 0.17:0.27:0.28, stirring for 12-48 hours at 20-40° C.; subjecting the reaction product to rotary evaporation, redissolution, extraction and drying; concentrating under reduced pressure; and triturating with an organic solvent to obtain an intermediate compound I; dissolving a compound having the structural formula C14-OH, intermediate compound I, and benzotriazol-1-yl-oxy-tris(pyrrolidino)phosphonium hexafluorophosphate in an organic solvent in a molar ratio of 0.31:0.052:0.047; adjusting the pH to 7-8; stirring at 20-40° C. for 1-12 hours; subjecting the reaction product to rotary evaporation, redissolution, extraction, and drying; and concentrating under reduced pressure to obtain a vincamine derivative having the structural formula A1-NH-B1-NH-C14;

Method II includes: when A is a group with the structural formula A1, B is a group of with the structural formula B5, and C is bovine serum albumin (BSA) having an amino acid sequence of SEQ ID NO: 1, dissolving a compound having the structural formula A1-OH, cystamine, and benzotriazol-1-yl-oxy-tris(pyrrolidino)phosphonium hexafluorophosphate in an organic solvent in a molar ratio of 0.15:0.23:0.23; adjusting the pH to 7-8 using an organic base or acid; stirring and reacting at 20-40° C. for 1-12 hours, triturating the reaction product with an organic solvent; drying under vacuum to obtain a crude product; dissolving the crude product in an organic solvent; adding an aqueous solution of tris(2-carboxyethyl)phosphine hydrochloride (TCEP) (the molar ratio of the compound A1-OH to TCEP hydrochloride is 0.15:0.17); stirring at 20-40° C. for 1-12 hours, concentrating under reduced pressure; adding water to precipitate the product; and drying to obtain an intermediate compound II; dissolving intermediate compound II and NHS-PEGₙ-Mal in an organic solvent in a molar ratio of 0.02:0.01; stirring the mixture at 20-40° C. for 1-12 hours; and adding this mixture to a PBS buffer solution of the bovine serum albumin (BSA) (amino acid sequence SEQ ID NO: 1), stirring at 20-40° C. for 1-12 hours; dialyzing against pure water; and freeze-drying to obtain a vincamine derivative having the structural formula (A1-NH-B5-NH)₁₁-C15;

Method III: a preparation method for vincamine derivatives with the other structural formulas includes the following steps:
(1) dissolving a compound having the structural formula C—Br, a compound having the structural formula HO—B—NH₂ or H₂N—B—NH₂, potassium carbonate, and potassium iodide in an organic solvent in a molar ratio of 1.21:0.552:2.43:0.552; heating to 45-65° C. for 12-48 hours; cooling and filtering the reaction mixture; extracting the filtrate with n-hexane; and purifying by silica gel chromatography to obtain an intermediate compound having the structural formula

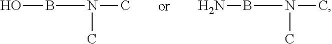

wherein B is selected from —(CH₂)ₙ—, —(CH₂)ₙ—S—S—(CH₂)ₙ—, or —(CH₂)ₙ-TK-(CH₂)ₙ—, and C is a group as defined above except for a hydroxyl-substituted linear alkyl group having 10 to 18 carbon atoms;
alternatively, dissolving an epoxy-substituted linear alkane group having 10 to 18 carbon atoms and a compound having the structural formula HO—B—NH₂ or H₂N—B—NH₂ in an organic solvent in a molar ratio of 1.26:0.57; heating to 55-75° C. for 12-48 hours; concentrating the reaction mixture under reduced pressure; and purifying by silica gel chromatography to obtain an intermediate compound having the structural formula

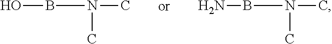

wherein C is a hydroxyl-substituted linear alkyl group having 10 to 18 carbon atoms;
(2) dissolving a compound having the structural formula A-OH, the intermediate compound having the structural formula

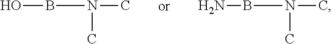

1-ethyl-3-(3-dimethylaminopropyl)carbodiimide hydrochloride (EDC·HCl) and 4-dimethylaminopyridine (DMAP) in an organic solvent in a molar ratio of 0.15:1.59:0.183:0.0081; heating to 20-40° C. for 24-96 hours; diluting the reaction mixture and extracting; drying the organic phase; concentrating under reduced pressure; and purifying by silica gel chromatography to obtain a vincamine derivative having the structural formula

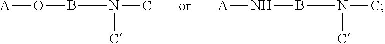

A method for preparing a vincamine derivative having the structural formula A-O—NH—C is as follows:
when A is a group A1 and C is a group C12 (DSPE-PEG-NH₂), dissolve a compound having the structural formula A1-OH, a compound having the structural formula C12-NH₂, and benzotriazol-1-yl-oxy-tris(pyrrolidino)phosphonium hexafluorophosphate in an organic solvent; stir at 20-40° C. for 24-96 hours; dialyze against pure water; and freeze-drying to obtain a vincamine derivative having the structural formula A1-O—NH-C12; and

when A is a group A1 and C is a group C13(DOX-NH₂), dissolve a compound having the structural formula A1-OH, a compound having the structural formula C13-NH₂ and benzotriazol-1-yl-oxy-tris(pyrrolidino)phosphonium hexafluorophosphate in an organic solvent; adjust the pH to 7-8; stir at 20-40° C. for 1-12 hours, subject the reaction product to rotary evaporation, redissolution, extraction and drying; concentrate under reduced pressure; and triturate with an organic solvent to obtain a vincamine derivative having the structural formula A1-O—NH-C13.

Preferably, the eluent used for silica gel chromatography in step (1) is a mixture of methanol and dichloromethane in a volume ratio ranging from 10:90 to 90:10, and the eluent used in step (2) is a mixture of acetone and n-hexane in a volume ratio ranging from 20:80 to 80:20.

Preferably, the organic solvent is selected from acetonitrile, ethanol, methanol, N,N-dimethylformamide, N,N-diisopropylethylamine, ethyl acetate, methyl tert-butyl ether, triethylamine, pyridine, or dichloromethane.

3. Use of the vincamine derivative in the brain-targeted delivery of drugs for treating brain diseases.

The present application has the following beneficial effects. The present application discloses a vincamine derivative and a preparation method thereof. The vincamine derivative has the following advantages: (i) Modification of the tail chain increases the lipophilicity of the vincamine compounds without compromising their inherent cerebral blood flow regulation ability, thereby facilitating the penetration of the carried drug across the blood-brain barrier, exerting a neuroprotective effect, and improving cerebral microcirculatory disorders. (ii) The tertiary amine group in the core structure of the vincamine derivative is ionizable under acidic conditions, enabling efficient delivery of nucleic acid drugs and lysosomal escape via charge-mediated adsorption, thus enhancing intracellular transport. (iii) The vincamine derivative retains the various pharmacological activities inherent to vincamine and exhibits high safety. Therefore, the vincamine derivative disclosed herein show good application prospects in the brain-targeted delivery of drugs for treating brain diseases.

Additional advantages, objects, and features of the present application are described in part in the following description and, in part, become apparent to those skilled in the art based on an examination of the following or may be learned from the practice of the present application. The objectives and other advantages of the present application are achieved and obtained through the following description.

### BRIEF DESCRIPTION OF THE DRAWINGS

To make the objectives, technical solutions and advantages of the present application more clear, the present application is described in detail below with reference to the accompanying drawings, in which:

FIG. 1 is a 1H NMR spectrum of compound 1 in Example 1;

FIG. 2 is a 1H NMR spectrum of compound 2 in Example 2;

FIG. 3 is a transmission electron micrograph of lipid nanoparticles (LNP) (compound 2 (A5-B2-C6)-14);

FIG. 4 shows pKa values of lipid nanoparticles prepared from different vincamine derivatives determined by TNS fluorescence method;

FIG. 5 shows the FAM fluorescence intensity in different administration groups (lipid nanoparticles prepared using different vincamine derivatives) observed under a fluorescence microscope;

FIG. 6 shows the cell viability calculated from the absorbance at 570 nm measured by a microplate reader for wells treated with lipid nanoparticles formed from different vincamine derivatives;

FIG. 7 shows the effects of lipid nanoparticles formed from different vincamine derivatives on cerebral microvascular blood flow in mice determined by a laser speckle imager;

FIG. 8 shows the N/P ratio of siRNA encapsulated in lipid nanoparticles formed from a vincamine derivative (A1-B1-C5) determined by gel retardation experiment;

FIG. 9 shows the endosomal escape effect of siRNA-encapsulated lipid nanoparticles formed from compound 1 (A1-B1-C5), compound 2 (A5-B2-C6) and compound 31 (A11-B3-C11) in Table 2, taken by confocal microscopy;

FIG. 10 shows the effect of lipid nanoparticles formed from compound 2 (A5-B2-C6) on cellular ROS levels detected by single cell analyzer;

FIG. 11 is a mass spectra of NH2-PEG2000-DSPE (a) and compound 35 prepared in Example 6 (b);

FIG. 12 is a mass spectrum of compound 36 prepared in Example 7;

FIG. 13 is a mass spectrum of compound 37 prepared in Example 7;

FIG. 14 shows the TLC verification result of compound 38 prepared in Example 8;

FIG. 15 is a standard curve obtained by measuring the absorbance after the reaction of L-leucine (0-10 mM) with OPA reagent; and

FIG. 16 shows the fluorescence images collected by the VISQUE in vivo Smart-LF System at predetermined time points (0.5, 1, and 2 hours) after compound 31, compound 35, compound 36, compound 37, and compound 38 (A1-B5-C15) are injected into mice.

### DESCRIPTION OF EMBODIMENTS

The following describes the embodiments of the present application through specific examples. Those skilled in the art may easily understand other advantages and effects of the present application from the contents disclosed in this specification. The present application may also be implemented or applied through other different specific implementations, and the details in this specification may also be modified or changed in various ways based on different viewpoints and applications without departing from the spirit of the present application. It should be noted that the illustrations provided in the following embodiments are only used to schematically illustrate the basic concept of the present application. The following embodiments and features therein may be combined with each other unless there is any conflict.

In the following examples, the related groups in the structural formulas and the structural formulas of the compounds involved are shown below:
A is any one of structural formulas A1 to A12:

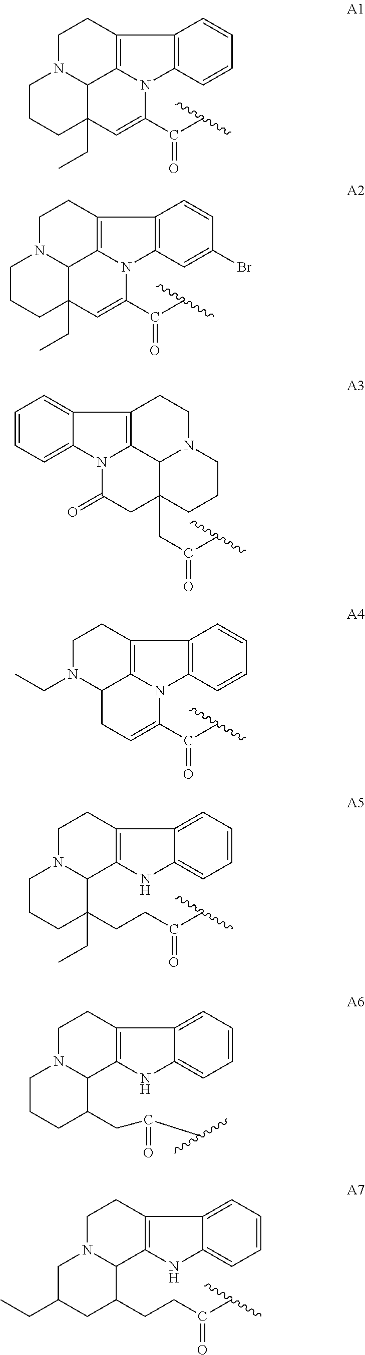

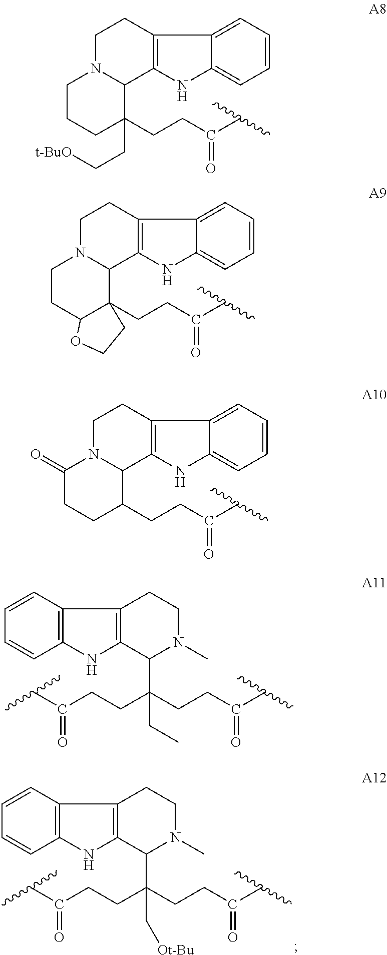

B is any one of B1 to B5, wherein B1 is —(CH₂)₂—, B2 is —(CH₂)₆—, B3 is —(CH₂)₂—S—S—(CH₂)₂—, B4 is —(CH₂)₂—S—C(CH₃)₂—S—(CH₂)₂—, and B5 is —(CH₂)₂—S-Mal-PEG-NHS—;
C or C′ is any one of structural formulas C1 to C14:

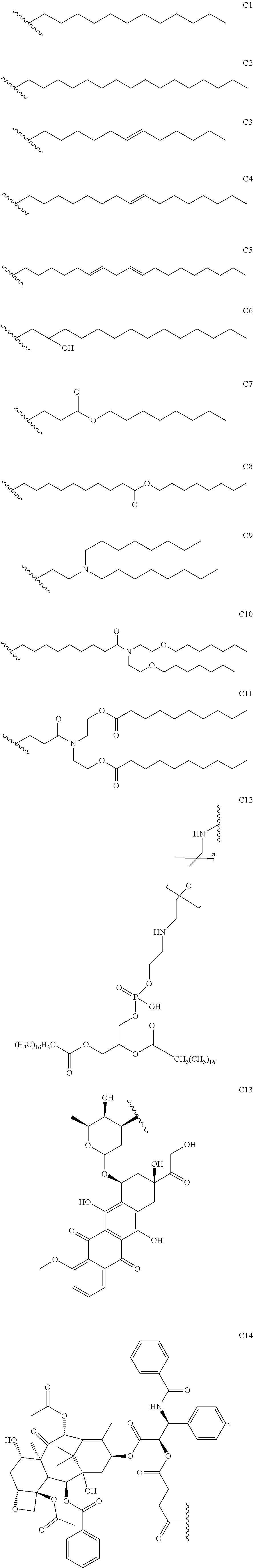

In addition, C also includes C15, wherein C15 is bovine serum albumin (BSA);

Compound 1 to compound 38 have the following structural formulas:

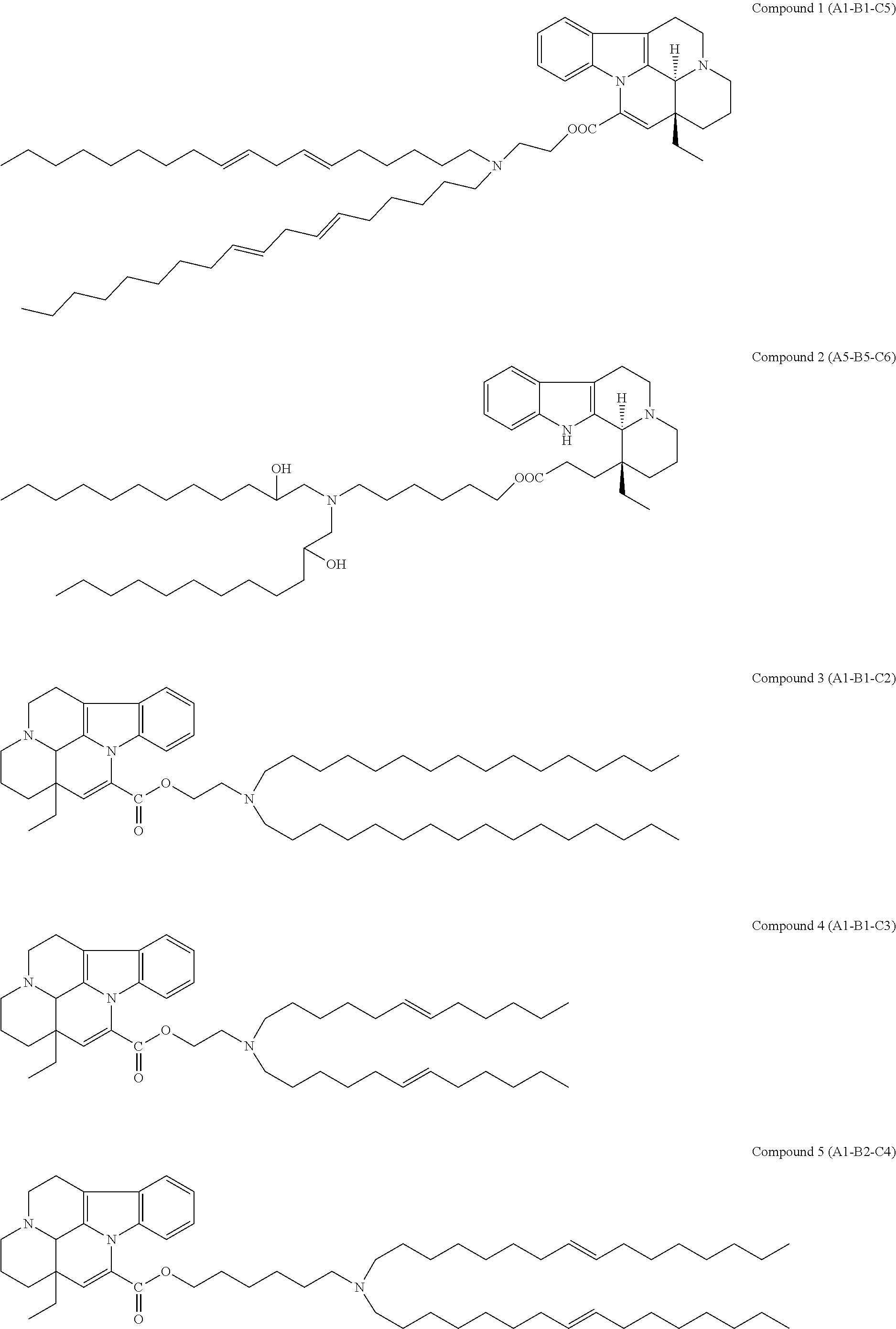

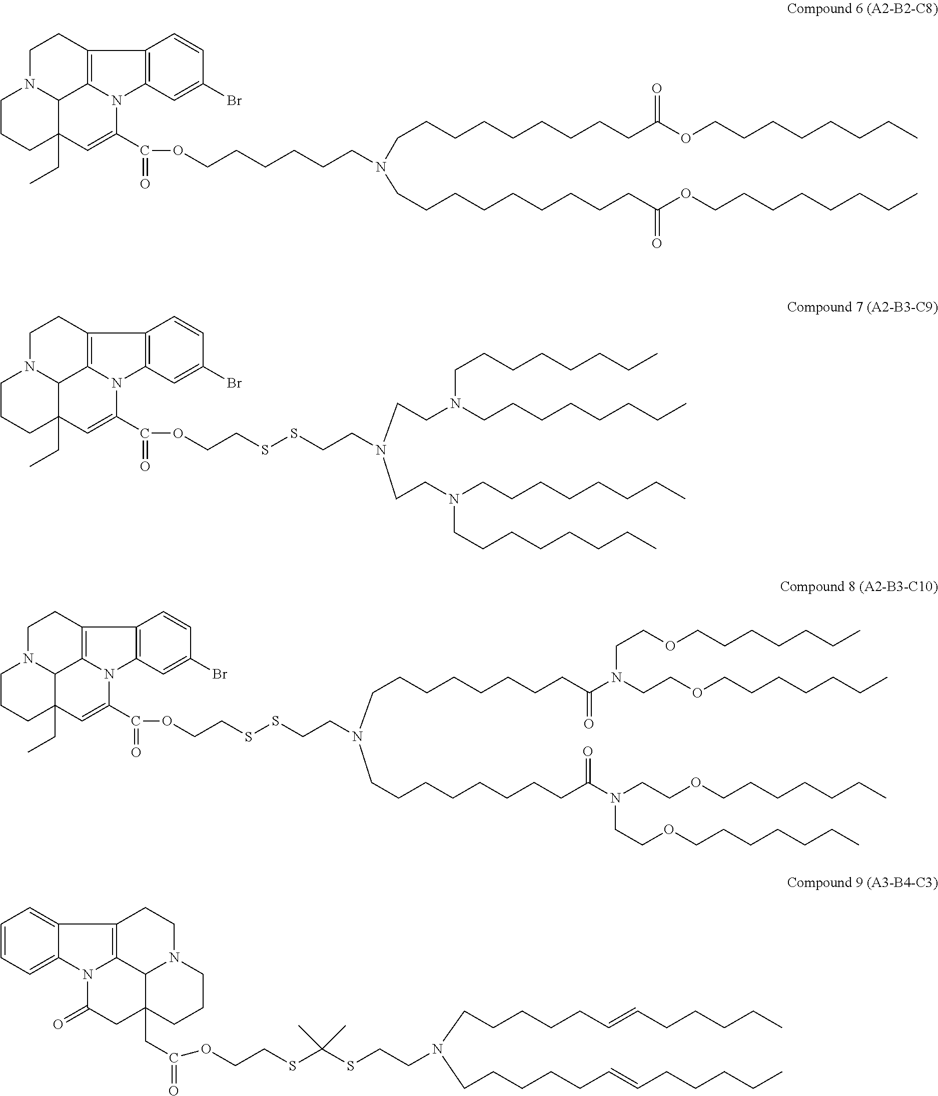

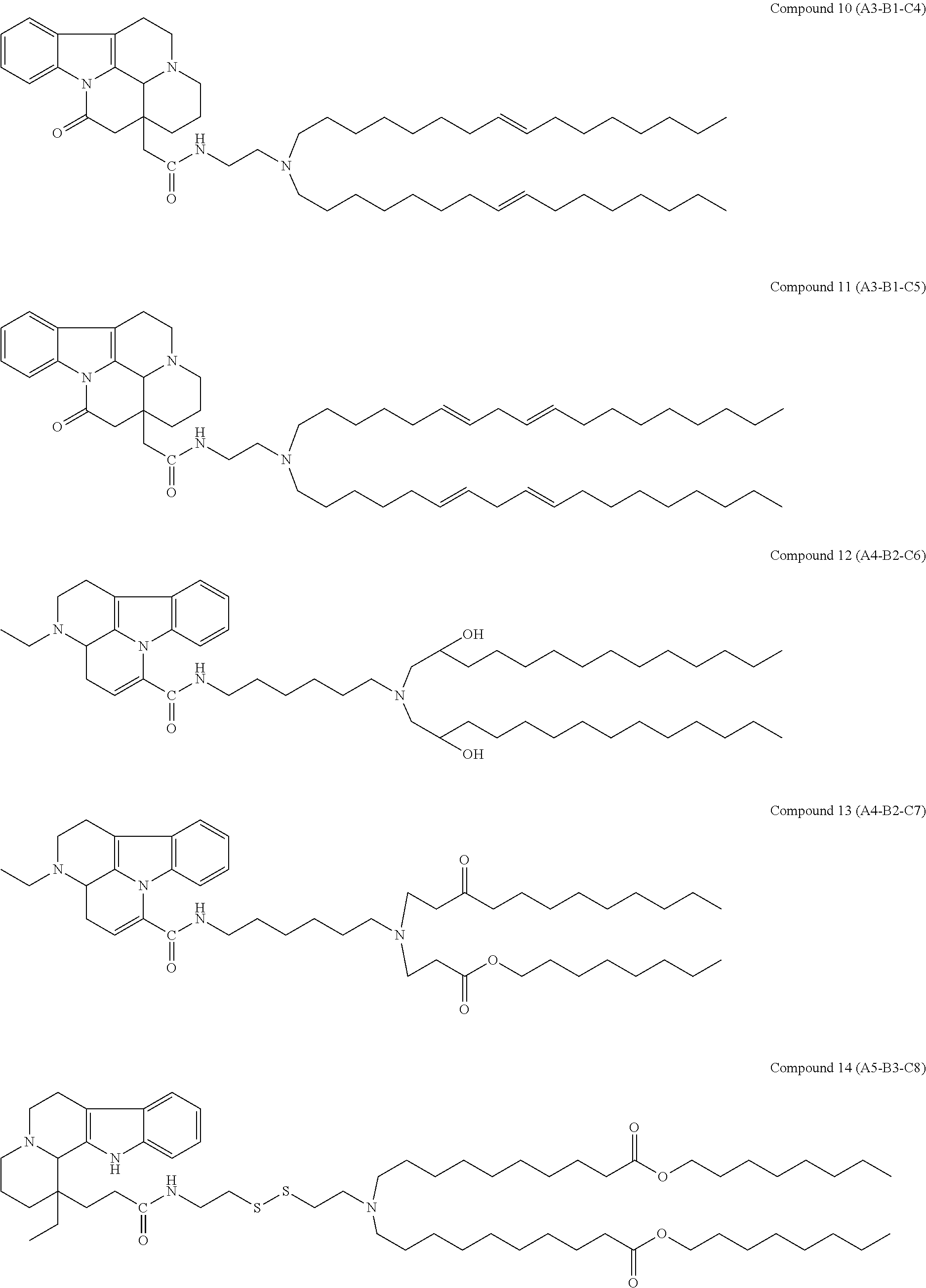

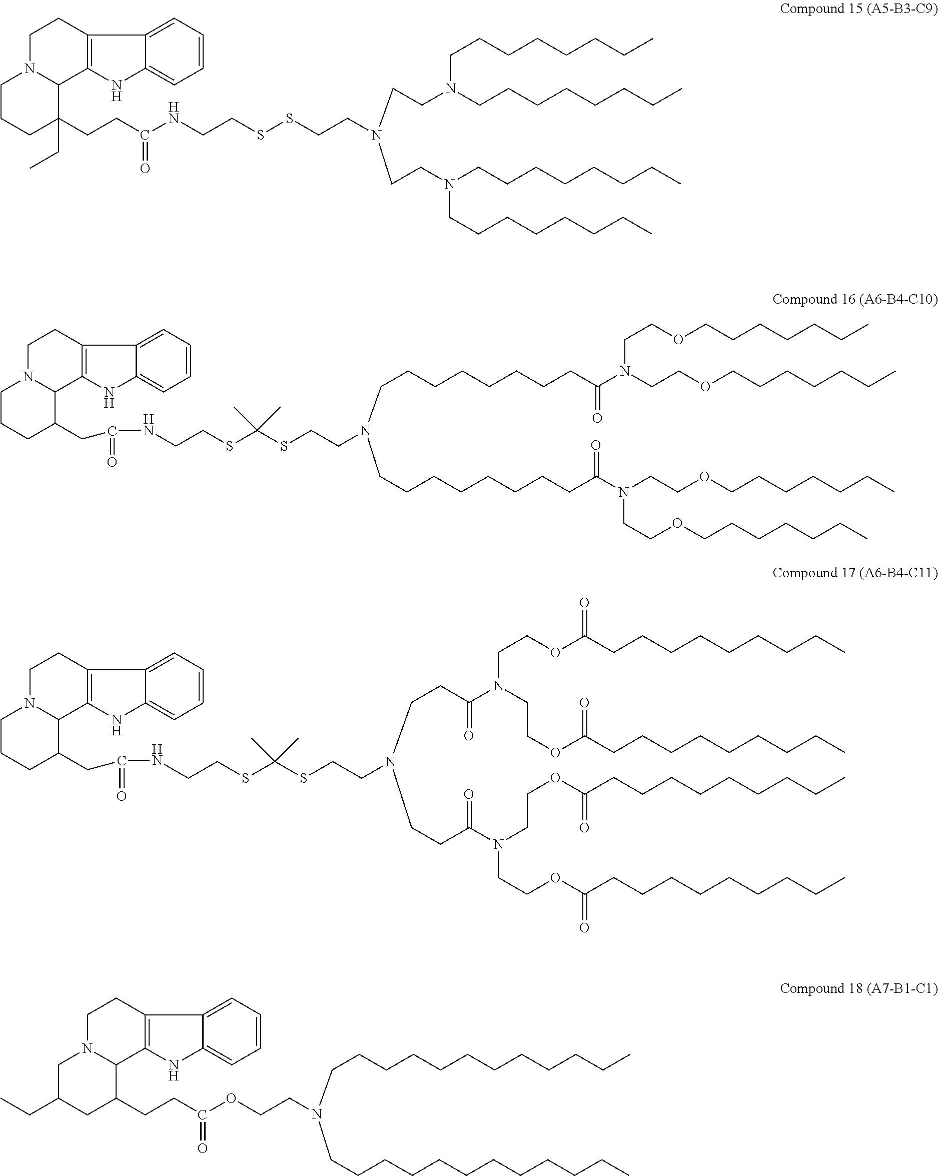

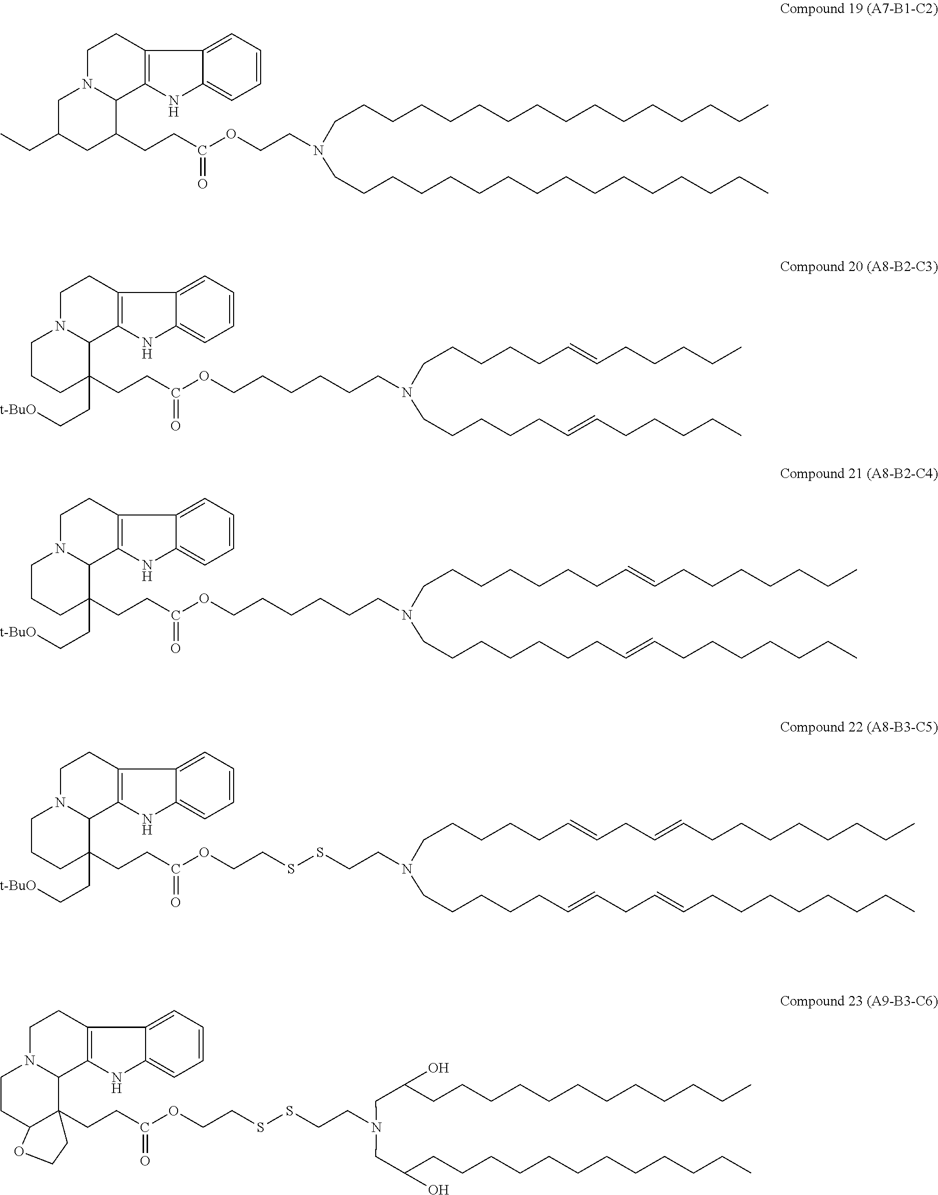

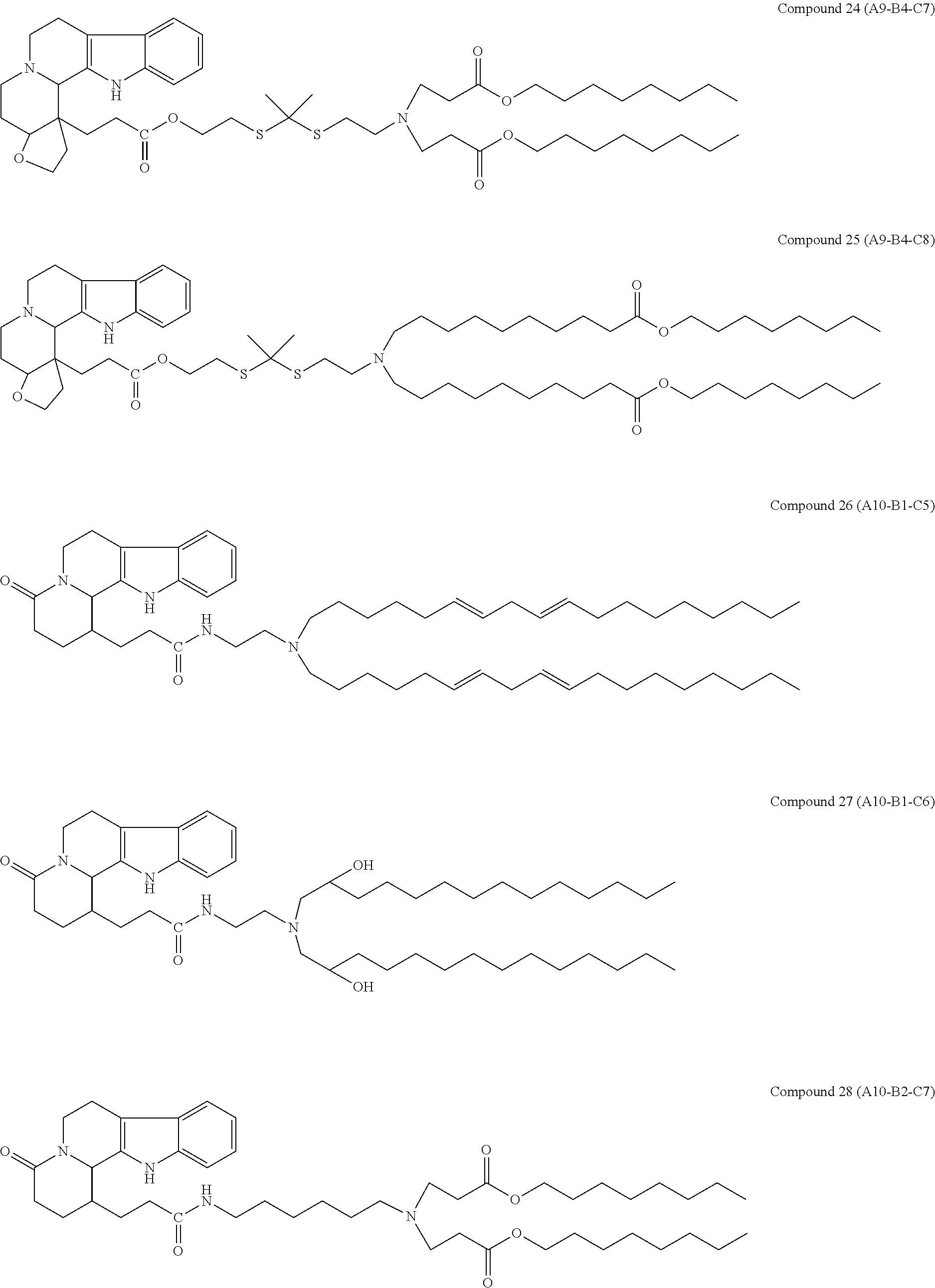

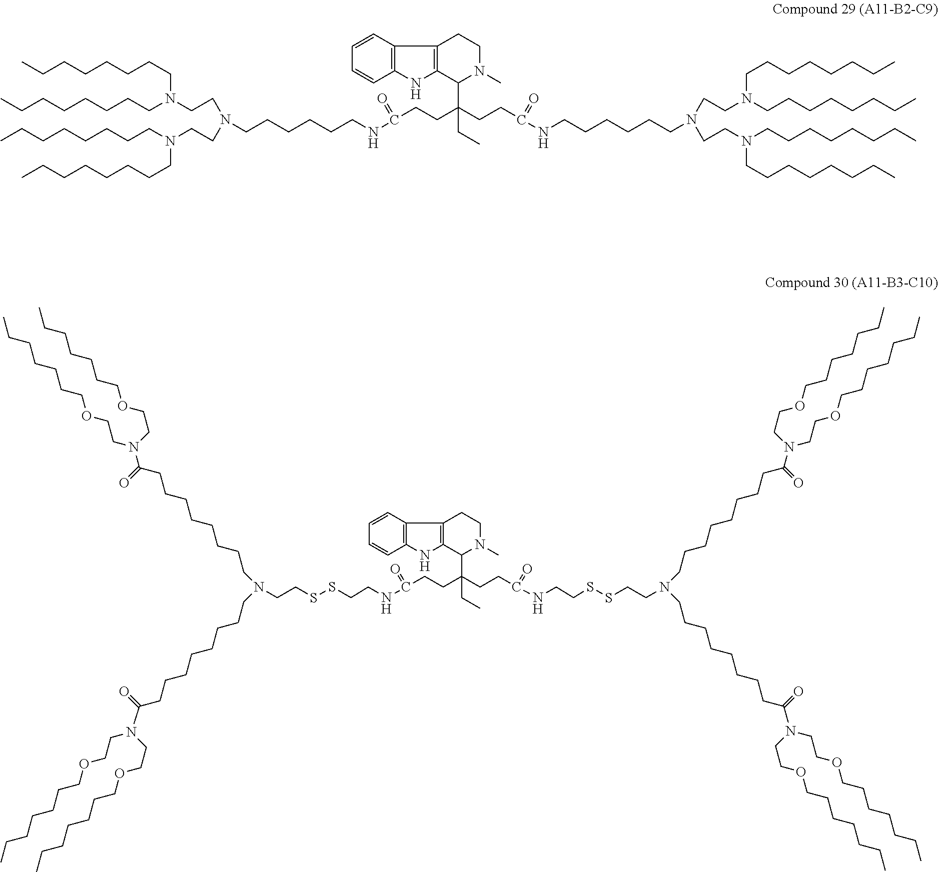

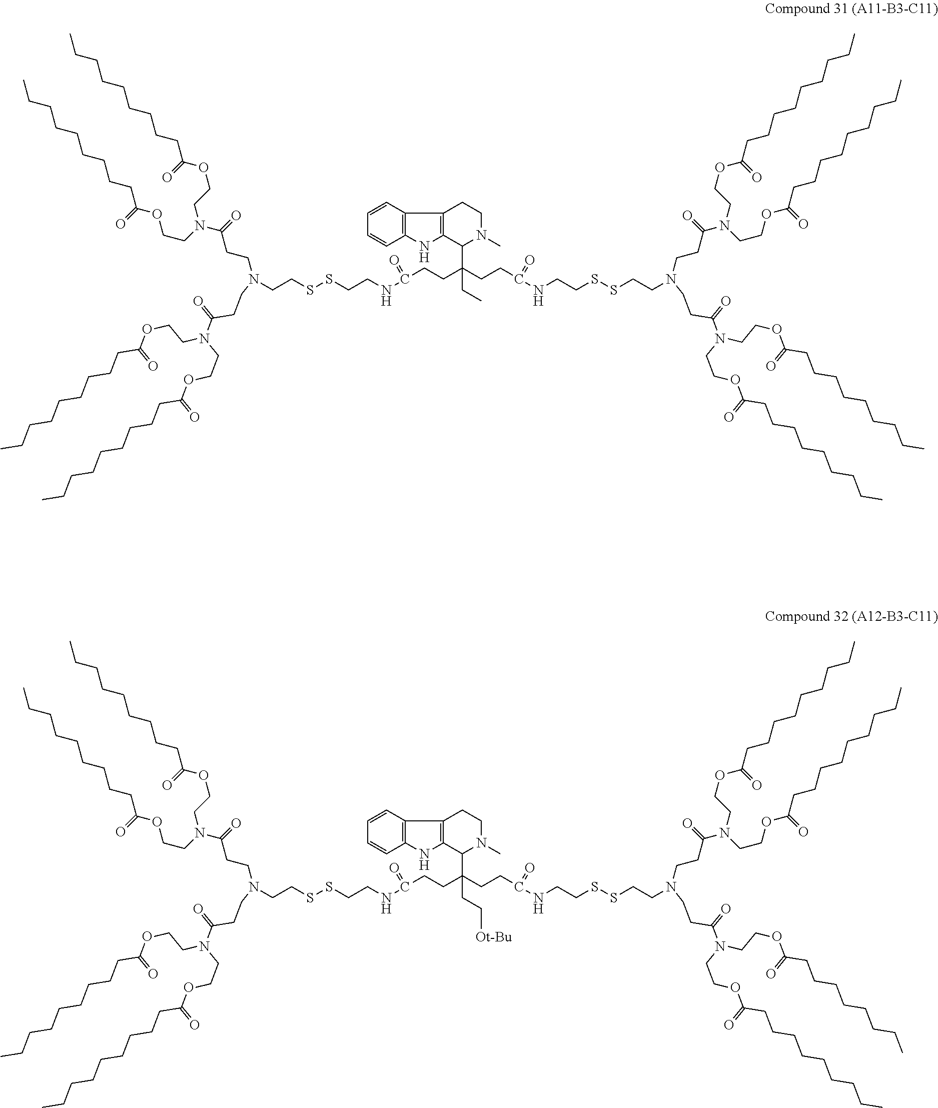

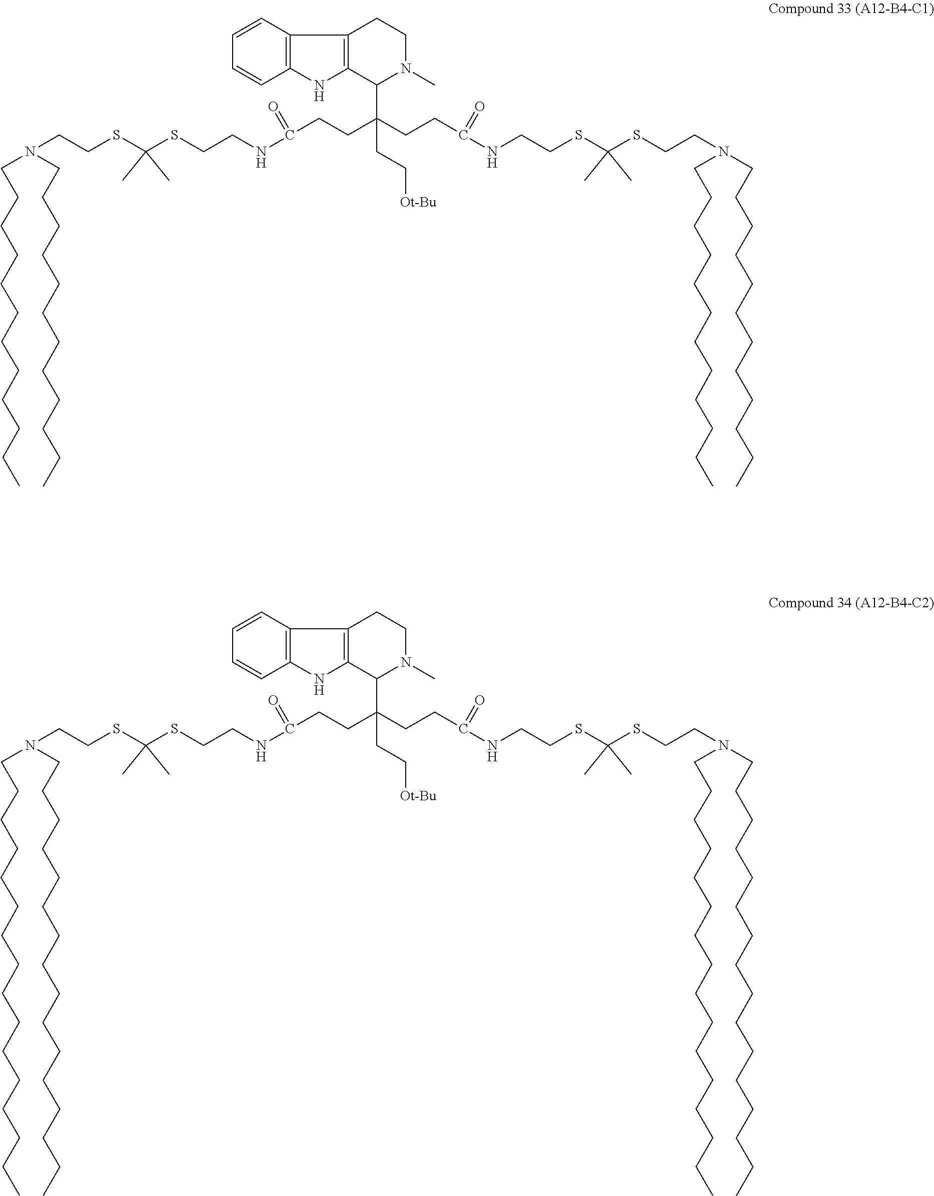

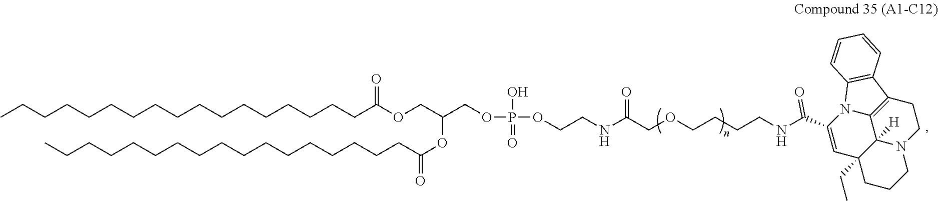

wherein n is a positive integer

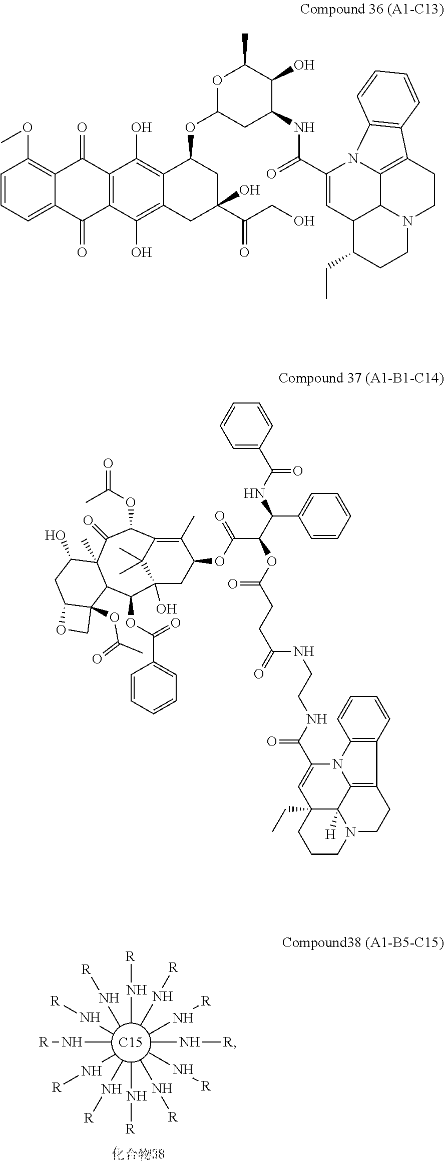

wherein the C15 is bovine serum albumin (BSA), R is

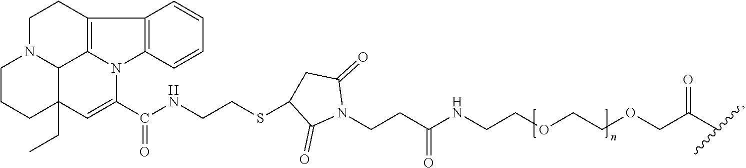

and n is a positive integer.

### Example 1

A vincamine derivative (compound 1) was prepared, the structural formula of which was:

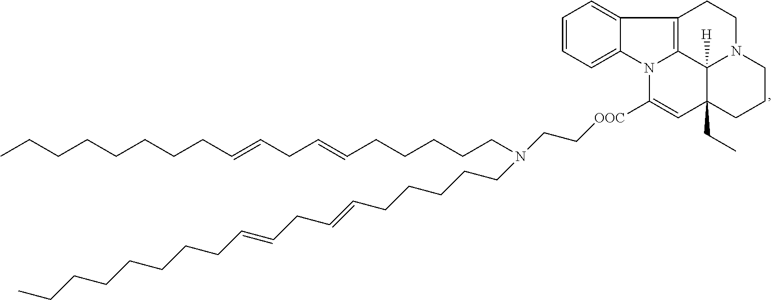

and the preparation method was specifically as follows:

(1) (6Z,9Z)-18-bromooctadeca-6,9-diene (C5-Br) (400 mg, 1.21 mmol), 2-aminoethanol (HO-B1-NH₂) (35 μL, 0.552 mmol), potassium carbonate (330 mg, 2.43 mmol), and potassium iodide (9 mg, 0.552 mmol) were dissolved in 5 mL of acetonitrile in a molar ratio of 1.21:0.552:2.43:0.552. The reaction mixture was heated and stirred at 65° C. for 24 hours.

(2) The reaction mixture was cooled to room temperature, the filter cake was washed three times with n-hexane, the filtrate obtained by filtration was extracted with n-hexane, concentrated under reduced pressure, and separated by silica gel chromatography (methanol/dichloromethane=15/85 (v/v)), and an intermediate compound with the structural formula

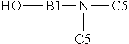

was obtained by rotary evaporation.

(3) A1-OH (50 mg, 0.15 mmol), the intermediate compound with the structural formula

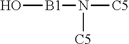

(88 mg, 1.59 mmol), 1-ethyl-3-(3-dimethylaminopropyl)carbodiimide hydrochloride (EDC·HCl, 35 mg, 0.183 mmol), and 4-dimethylaminopyridine (DMAP, 1 mg, 0.0081 mmol) were dissolved in 6 mL of pyridine/dichloromethane (1/1, v/v). The reaction mixture was stirred at room temperature for 24 hours.

(4) The organic phase was diluted with dichloromethane (6 mL), washed four times with 5 mL of 2 M hydrochloric acid solution and saturated brine, then dried over sodium sulfate, concentrated under reduced pressure, and separated by silica gel chromatography (acetone/n-hexane=1/1 (v/v); dichloromethane/methanol=1/3 (v/v)). The final product was obtained by rotary evaporation to obtain the vincamine derivative (compound 1) with the structural formula

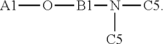

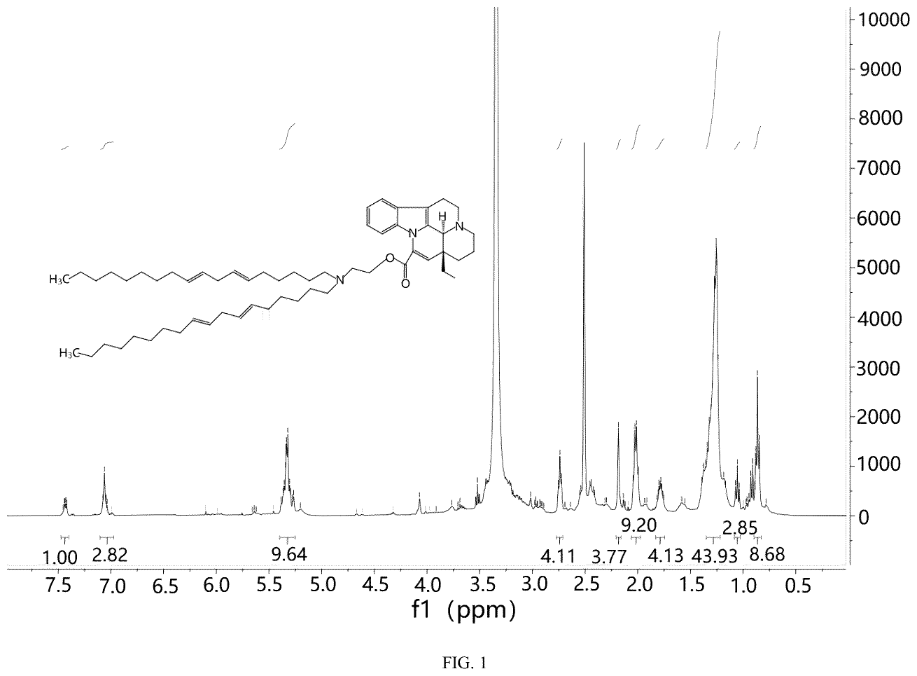

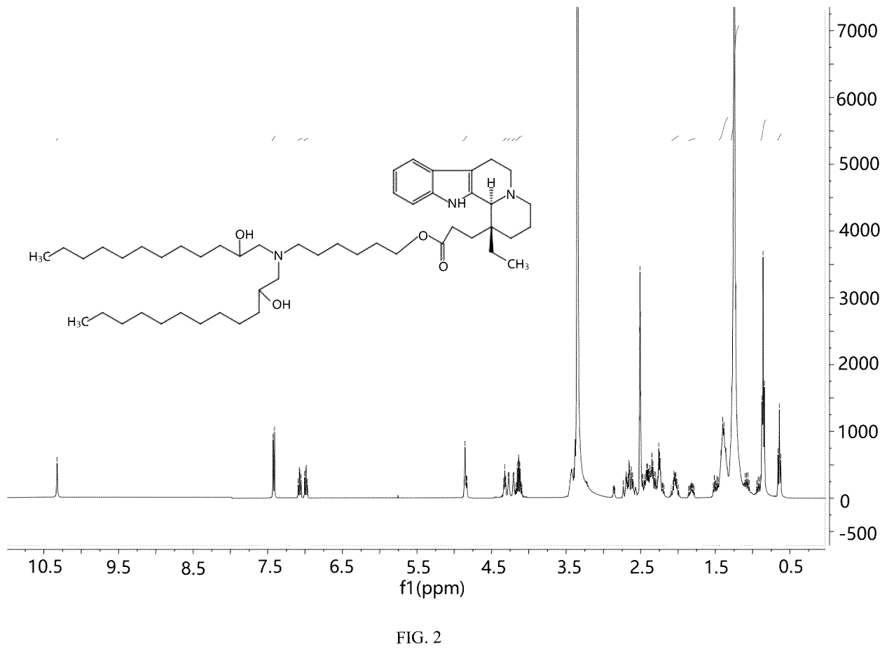

The ¹H NMR (400 MHz, DMSO-d₆) spectrum of this vincamine derivative is shown in FIG. 1.

### Example 2

A vincamine derivative (compound 2) was prepared, the structural formula of which was:

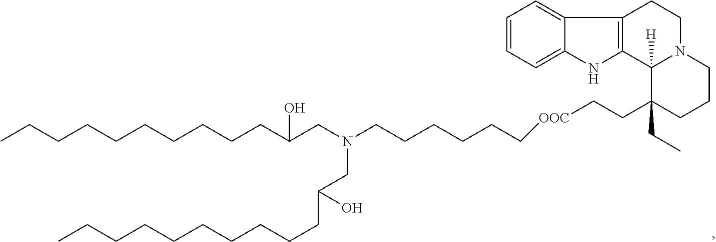

and the preparation method was specifically as follows:

(1) 1,2-Epoxydodecane (C6-epoxy 233 mg, 1.26 mmol) and 6-aminohexanol (HO-B2-NH₂, 202 mg, 0.57 mmol) were dissolved in 2 mL of ethanol in a molar ratio of 1.26:0.57. The reaction mixture was heated and stirred at 75° C. for 24 hours.

(2) The reaction product was concentrated under reduced pressure and separated by silica gel chromatography (methanol/dichloromethane=1/30 (v/v)), and rotary evaporated to obtain an intermediate compound with the structural formula

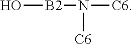

(3) A5-OH (50 mg, 0.15 mmol), the intermediate compound with the structural formula

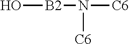

(88 m, 1.59 mmol), 1-ethyl-3-(3-dimethylaminopropyl)carbodiimide hydrochloride (EDC·HCl, 35 mg, 0.183 mmol), and 4-dimethylaminopyridine (DMAP, 1 mg, 0.0081 mmol) were dissolved in 6 mL of pyridine/dichloromethane (1/1, v/v). The reaction mixture was stirred at room temperature for 24 hours.

(4) The organic phase was diluted with dichloromethane (6 mL), washed four times with 2 M hydrochloric acid solution (4×5 mL) and saturated brine, then dried over sodium sulfate, concentrated under reduced pressure, and separated by silica gel chromatography (acetone/n-hexane=1/1 (v/v); dichloromethane/methanol=1/3 (v/v)). The final product was obtained by rotary evaporation to obtain the vincamine derivative with the structural formula

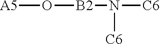

(compound 2). ¹H NMR (400 MHz, DMSO-d6) δ 10.32 (s, 1H), 7.42 (d, J=8.0 Hz, 2H), 7.07 (dd, J=8.4, 6.9 Hz, 1H), 6.99 (dd, J=8.1, 6.8 Hz, 1H), 4.84 (d, J=10.1 Hz, 2H), 4.32 (t, J=5.1 Hz, 1H), 4.27 (d, J=3.7 Hz, 1H), 4.21 (d, J=3.9 Hz, 1H), 4.18-4.09 (m, 2H), 2.09-1.98 (m, 2H), 1.82 (ddd, J=13.7, 10.3, 6.1 Hz, 1H), 1.39 (q, J=9.7 Hz, 10H), 1.29-1.18 (m, 50H), 0.86 (t, J=6.7 Hz, 9H), 0.64 (t, J=7.5 Hz, 3H).

### Example 3

According to the reaction steps in Example 1 (i.e., certain reactants in Example 1 were replaced, and the addition ratio and other reaction conditions were the same as those in Example 1), the following compounds were prepared:

(1) Compound 3 (A1-B1-C2): Compound 3 with the structural formula

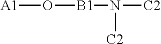

was prepared by replacing (6Z,9Z)-18-bromooctadeca-6,9-diene (C5-Br) with 16-bromohexadecane (C2-Br) and replacing 2-aminoethanol (HO-B1-NH₂) with 2-aminoethanol (HO-B1-NH₂). ¹H NMR (400 MHz, DMSO-d6) δ7.62 (d, 1H), 7.51 (d, 1H), 7.46 (t, 1H), 7.17 (t, 1H), 6.74 (s, 1H), 4.27 (t, 2H), 3.63 (s, 1H), 2.90 (m, 6H), 2.68 (m, 6H), 1.64 (m, 2H), 1.48-1.33 (m, 60H), 0.89 (m, 3H), 0.84 (m, 6H).

(2) Compound 4 (A1-B1-C3): Compound 4 with the structural formula

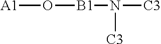

was prepared by replacing (6Z,9Z)-18-bromooctadeca-6,9-diene (C5-Br) with (6Z)-12-bromododec-6-ene (C3-Br). ¹H NMR (400 MHz, DMSO-d6) δ 7.62 (d, 1H), 7.52 (d, 1H), 7.46 (t, 1H), 7.17 (t, 1H), 6.71 (s, 1H), 5.39 (m, 4H), 4.26 (t, 2H), 3.59 (s, 1H), 2.90 (m, 6H), 2.68 (m, 6H), 2.00 (m, 8H), 1.51 (m, 4H), 1.48-1.33 (m, 26H), 0.89 (m, 9H).

(3) Compound 5 (A1-B2-C4): Compound 5 with the structural formula

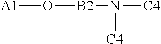

was obtained by replacing (6Z,9Z)-18-bromooctadeca-6,9-diene (C5-Br) with (9Z)-16-bromohexadec-9-ene (C4-Br), replacing 2-aminoethanol (HO-B1-NH₂) with 6-aminohexanol (HO-B2-NH₂), and replacing A1-OH with A2-OH. ¹H NMR (400 MHz, DMSO-d6) δ 7.62 (d, 1H), 7.52 (d, 1H), 7.46 (t, 1H), 7.17 (t, 1H), 6.71 (s, 1H), 5.39 (m, 4H), 4.20 (t, 2H), 3.59 (s, 1H), 2.68 (m, 4H), 2.68 (m, 6H), 1.99 (m, 8H), 1.49 (m, 6H), 1.49-1.29 (m, 50H), 0.89 (m, 9H).

(4) Compound 6 (A2-B2-C8): Compound 6 with the structural formula

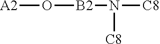

was obtained by replacing (6Z,9Z)-18-bromooctadeca-6,9-diene (C5-Br) with octyl 10-bromodecanoate (C8-Br), replacing 2-aminoethanol (HO-B1-NH₂) with 6-aminohexanol (HO-B2-NH₂), and replacing A1-OH with A2-OH. ¹H NMR (400 MHz, DMSO-d6) δ 7.84 (s, 1H), 7.54 (d, 1H), 7.47 (d, 1H), 6.74 (s, 1H), 4.20 (t, 4H), 4.02 (t, 2H), 3.62 (s, 1H), 2.90 (t, 6H), 2.36 (m, 10H), 1.69-1.29 (m, 66H), 0.89 (m, 9H).

(5) Compound 7 (A2-B3-C9): Compound 7 with the structural formula

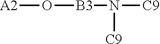

was obtained by replacing (6Z,9Z)-18-bromooctadeca-6,9-diene (C5-Br) with N,N-dioctyl-2-bromo-ethylamine (C9-Br), replacing 2-aminoethanol (HO-B1-NH₂) with 6-amino-3,4-dithia-hexanol (HO-B3-NH₂), and replacing A1-OH with A2-OH. ¹H NMR (400 MHz, DMSO-d6) δ 7.84 (s, 1H), 7.54 (d, 1H), 7.47 (d, 1H), 6.74 (s, 1H), 4.31 (t, 2H), 3.80 (d, 1H), 3.07 (t, 12H), 2.87 (t, 2H), 2.73-2.59 (m, 10H), 2.53 (m, 8H), 1.70-1.32 (m, 50H), 0.89 (m, 15H).

(6) Compound 8 (A2-B3-C10): Compound 8 with the structural formula

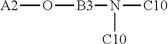

was obtained by replacing (6Z,9Z)-18-bromooctadeca-6,9-diene (C5-Br) with N,N-bis(3-oxodecyl)nonanamide (C10-Br), replacing 6-amino-3,4-dithia-hexanol (HO-B3-NH₂) with 2-aminoethanol (HO-B1-NH₂), and replacing A1-COOH with A2-OH. ¹H NMR (400 MHz, DMSO-d6) δ 7.84 (s, 1H), 7.54 (d, 1H), 7.47 (d, 1H), 6.74 (s, 1H), 4.01 (t, 2H), 3.80 (t, 8H), 3.54 (s, 1H), 3.28 (m, 16H), 3.07 (t, 4H), 2.87 (t, 2H), 2.73-2.54 (m, 10H), 2.35 (t, 4H), 1.52-1.32 (m, 70H), 0.89 (m, 15H).

(7) Compound 9 (A3-B4-C3): Compound 9 with the structural formula

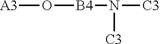

was obtained by replacing (6Z,9Z)-18-bromooctadeca-6,9-diene (C5-Br) with (6Z)-12-bromododec-6-ene (C3-Br), replacing 2-aminoethanol (HO-B1-NH₂) with 4,4-dimethyl-3,5-thioketal-heptanol (HO-B4-NH₂), and replacing A1-OH with A3-OH. ¹H NMR (400 MHz, DMSO-d6) δ 7.74 (d, 1H), 7.61 (d, 1H), 7.44 (t, 1H), 7.16 (t, 1H), 5.48 (m, 4H), 4.30 (t, 2H), 4.15 (s, 1H), 3.04 (t, 4H), 2.77-2.02 (m, 26H), 1.70-1.29 (m, 32H), 0.89 (m, 6H).

(8) Compound 10 (A3-B1-C4): Compound 10 of the structural formula

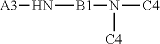

was obtained by replacing (6Z,9Z)-18-bromooctadeca-6,9-diene (C5-Br) with (9Z)-16-bromohexadec-9-ene (C4-Br), replacing 2-aminoethanol (HO-B1-NH₂) with ethylenediamine (NH₂—B1-NH₂), and replacing A1-OH with A3-OH. ¹H NMR (400 MHz, DMSO-d6) δ7.74 (s, 1H), 7.61 (d, 1H), 7.44 (d, 1H), 7.16 (t, 1H), 6.89 (t, 1H), 5.48 (m, 4H), 4.18 (s, 1H), 3.20 (t, 2H), 3.08 (t, 4H), 2.74-1.94 (m, 20H), 1.57-1.28 (m, 44H), 0.89 (m, 6H).

(9) Compound 11 (A3-B1-C5): Compound 11 with the structural formula

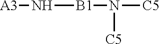

was obtained by replacing 2-aminoethanol (HO-B1-NH₂) with ethylenediamine (NH₂—B1-NH₂) and replacing A1-OH with A3-OH. ¹H NMR (400 MHz, DMSO-d6) δ7.74 (s, 1H), 7.61 (d, 1H), 7.44 (d, 1H), 7.16 (t, 1H), 6.89 (t, 1H), 5.55-5.11 (m, 8H), 4.18 (s, 1H), 3.31 (t, 2H), 3.08 (t, 4H), 2.82 (m, 4H), 2.70-1.94 (m, 20H), 1.53-1.26 (m, 40H), 0.89 (m, 6H).

(10) Compound 13 (A4-B2-C7): Compound 13 with the structural formula

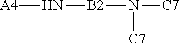

was obtained by replacing (6Z,9Z)-18-bromooctadeca-6,9-diene (C5-Br) with octyl 3-bromopropanoate (C7-Br), replacing 2-aminoethanol (HO-B1-NH₂) with hexamethylenediamine (NH₂—B2-NH₂), and replacing A1-OH with A4-OH. ¹H NMR (400 MHz, DMSO-d6) δ7.73 (s, 1H), 7.61 (d, 1H), 7.51 (d, 1H), 7.44 (t, 1H), 7.16 (t, 1H), 6.75 (t, 1H), 4.38 (t, 4H), 4.15 (t, 1H), 3.72 (t, 4H), 3.22 (t, 2H), 3.03 (t, 2H), 2.82-2.53 (m, 12H), 1.54-1.27 (m, 32H), 1.14 (t, 3H), 0.89 (m, 6H).

(11) Compound 14 (A5-B3-C8): Compound 14 with the structural formula

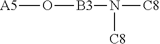

was obtained by replacing (6Z,9Z)-18-bromooctadeca-6,9-diene (C5-Br) with octyl 10-bromodecanoate (C8-Br), replacing 2-aminoethanol (HO-B1-NH₂) with 3,4-dithia-1,6-hexanediamine (HO-B3-NH₂), and replacing A1-OH with A5-OH. ¹H NMR (400 MHz, DMSO-d6) δ 9.20 (s, 1H), 7.54 (s, 1H), 7.30 (d, 1H), 7.23 (d, 1H), 7.16 (t, 1H), 6.79 (t, 1H), 4.38 (t, 4H), 4.14 (s, 1H), 3.40 (t, 2H), 3.05 (t, 4H), 2.79-2.28 (m, 16H), 2.02 (m, 2H), 1.62-1.27 (m, 60H), 0.89 (m, 6H), 0.85 (m, 3H).

(12) Compound 15 (A5-B3-C9): Compound 15 with the structural formula

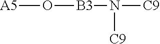

was obtained by replacing (6Z,9Z)-18-bromooctadeca-6,9-diene (C5-Br) with N,N-dioctyl-2-bromo-ethylamine (C9-Br), replacing 2-aminoethanol (HO-B1-NH₂) with 3,4-dithia-1,6-hexanediamine (HO-B3-NH₂), and replacing A1-OH with A5-OH. ¹H NMR (400 MHz, DMSO-d6) δ 9.20 (s, 1H), 7.54 (s, 1H), 7.30 (d, 1H), 7.23 (d, 1H), 7.16 (t, 1H), 6.79 (t, 1H), 3.90 (t, 2H), 3.40 (s, 1H), 3.05 (t, 8H), 2.79-2.30 (m, 20H), 2.02 (t, 2H), 1.77-1.26 (m, 56H), 0.89 (m, 12H), 0.85 (m, 3H).

(13) Compound 16 (A6-B4-C10): Compound 16 with the structural formula

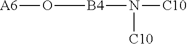

was obtained by replacing (6Z,9Z)-18-bromooctadeca-6,9-diene (C5-Br) with N,N-bis(3-oxodecyl)nonanamide (C10-Br), replacing 2-aminoethanol (HO-B1-NH₂) with 4,4-dimethyl-3,5-thioketal-1,7-heptanediamine (HO-B4-NH₂), and replacing A1-OH with A6-OH. ¹H NMR (400 MHz, DMSO-d6) δ 9.31 (s, 1H), 7.54 (s, 1H), 7.30 (d, 1H), 7.23 (d, 1H), 7.16 (t, 1H), 6.64 (t, 1H), 4.26 (t, 8H), 3.54 (m, 3H), 3.28 (m, 16H), 3.07 (t, 4H), 2.77-2.17 (m, 19H), 1.73-1.27 (m, 74H), 0.89 (m, 12H).

(14) Compound 17 (A6-B4-C11): Compound 17 with the structural formula

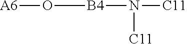

was obtained by replacing (6Z,9Z)-18-bromooctadeca-6,9-diene (C5-Br) with N,N-bis(3-acetyl-nonyl)-propionamide (C11-Br), replacing 2-aminoethanol (HO-B1-NH₂) with 4,4-dimethyl-3,5-thioketal-1,7-heptanediamine (HO-B4-NH₂), and replacing A1-OH with A6-OH. ¹H NMR (400 MHz, DMSO-d6) δ 9.31 (s, 1H), 7.54 (s, 1H), 7.30 (d, 1H), 7.23 (d, 1H), 7.16 (t, 1H), 6.64 (t, 1H), 4.56 (t, 8H), 3.54 (m, 14H), 2.84-2.17 (m, 28H), 1.73-1.26 (m, 66H), 0.89 (m, 12H).

(15) Compound 18 (A7-B1-C1): Compound 18 with the structural formula

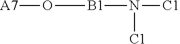

was prepared by replacing (6Z,9Z)-18-bromooctadeca-6,9-diene (C5-Br) with 12-bromododecane (C1-Br) and replacing A1-OH with A7-OH. ¹H NMR (400 MHz, DMSO-d6) δ 9.38 (s, 1H), 7.54 (d, 1H), 7.30 (d, 1H), 7.23 (t, 1H), 7.16 (t, 1H), 4.27 (t, 2H), 3.59 (d, 1H), 3.09 (m, 6H), 2.65-2.13 (m, 8H), 1.97 (m, 2H), 1.57-1.21 (m, 46H), 0.89 (m, 9H).

(16) Compound 19 (A7-B1-C2): Compound 19 with the structural formula

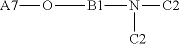

was prepared by replacing (6Z,9Z)-18-bromooctadeca-6,9-diene (C5-Br) with 16-bromohexadecane (C2-Br) and replacing A1-OH with A7-OH. ¹H NMR (400 MHz, DMSO-d6) δ 9.38 (s, 1H), 7.54 (d, 1H), 7.30 (d, 1H), 7.23 (t, 1H), 7.16 (t, 1H), 4.27 (t, 2H), 3.59 (d, 1H), 3.09 (m, 6H), 2.65-2.13 (m, 8H), 1.97 (m, 2H), 1.57-1.21 (m, 62H), 0.89 (m, 9H).

(17) Compound 20 (A8-B2-C3): Compound 20 with the structural formula

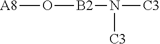

was obtained by replacing (6Z,9Z)-18-bromooctadeca-6,9-diene (C5-Br) with (6Z)-12-bromododec-6-ene (C3-Br), replacing 2-aminoethanol (HO-B1-NH₂) with 6-aminohexanol (HO-B2-NH₂), and replacing A1-OH with A8-OH. ¹H NMR (400 MHz, DMSO-d6) δ 9.27 (s, 1H), 7.54 (d, 1H), 7.30 (d, 1H), 7.23 (t, 1H), 7.16 (t, 1H), 5.48 (m, 4H), 4.14 (t, 2H), 3.90 (s, 1H), 3.48 (t, 2H), 3.05 (t, 6H), 2.74-2.18 (m, 16H), 1.78-1.29 (m, 40H), 1.16 (s, 9H), 0.89 (m, 6H).

(18) Compound 21 (A8-B2-C4): Compound 21 with the structural formula

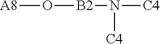

was obtained by replacing (6Z,9Z)-18-bromooctadeca-6,9-diene (C5-Br) with (9Z)-16-bromohexadec-9-ene (C4-Br), replacing 2-aminoethanol (HO-B1-NH₂) with 6-aminohexanol (HO-B2-NH₂), and replacing A1-OH with A8-OH. ¹H NMR (400 MHz, DMSO-d6) δ 9.27 (s, 1H), 7.54 (d, 1H), 7.30 (d, 1H), 7.23 (t, 1H), 7.16 (t, 1H), 5.48 (m, 4H), 4.14 (t, 2H), 3.90 (s, 1H), 3.48 (t, 2H), 3.05 (t, 6H), 2.74-2.18 (m, 16H), 1.78-1.29 (m, 56H), 1.16 (s, 9H), 0.89 (m, 6H).

(19) Compound 22 (A8-B3-C5): Compound 22 with the structural formula

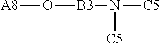

was obtained by replacing (6Z,9Z)-18-bromooctadeca-6,9-diene (C5-Br) with (6Z,9Z)-18-bromooctadeca-6,9-diene (C5-Br), replacing 2-aminoethanol (HO-B1-NH₂) with 6-amino-3,4-dithiahexan-1-ol (HO-B3-NH₂), and replacing A1-OH with A8-OH. ¹H NMR (400 MHz, DMSO-d6) δ 9.27 (s, 1H), 7.54 (d, 1H), 7.30 (d, 1H), 7.23 (t, 1H), 7.16 (t, 1H), 5.48 (m, 4H), 4.14 (t, 2H), 3.90 (s, 1H), 3.48 (t, 8H), 3.05 (t, 6H), 2.74-2.18 (m, 16H), 1.78-1.29 (m, 50H), 1.16 (s, 9H), 0.89 (m, 6H).

(20) Compound 24 (A9-B4-C7): Compound 24 with the structural formula

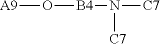

was obtained by replacing (6Z,9Z)-18-bromooctadeca-6,9-diene (C5-Br) with octyl 3-bromopropanoate (C7-Br), replacing 2-aminoethanol (HO-B1-NH₂) with 4,4-dimethyl-3,5-thioketal-heptanol (HO-B4-NH₂), and replacing A1-OH with A9-OH. ¹H NMR (400 MHz, DMSO-d6) δ 9.25 (s, 1H), 7.54 (d, 1H), 7.30 (d, 1H), 7.23 (t, 1H), 7.16 (t, 1H), 4.15 (t, 4H), 4.04 (t, 2H), 3.77 (m, 6H), 3.63 (m, 2H), 2.74-2.30 (m, 18H), 1.77-1.27 (m, 36H), 0.89 (m, 6H).

(21) Compound 25 (A9-B4-C8): Compound 25 with the structural formula

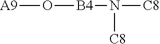

was obtained by replacing (6Z,9Z)-18-bromooctadeca-6,9-diene (C5-Br) with octyl 10-bromodecanoate (C8-Br), replacing 2-aminoethanol (HO-B1-NH₂) with 4,4-dimethyl-3,5-thioketal-heptanol (HO-B4-NH₂), and replacing A1-OH with A9-OH. ¹H NMR (400 MHz, DMSO-d6) δ 9.25 (s, 1H), 7.54 (d, 1H), 7.30 (d, 1H), 7.23 (t, 1H), 7.16 (t, 1H), 4.15 (t, 4H), 4.04 (t, 2H), 3.77 (m, 6H), 3.63 (m, 2H), 2.74-2.30 (m, 18H), 1.77-1.27 (m, 64H), 0.89 (m, 6H).

(22) Compound 26 (A10-B1-C5): Compound 26 of the structural formula

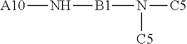

was obtained by replacing (6Z,9Z)-18-bromooctadeca-6,9-diene (C5-Br) with (6Z,9Z)-18-bromooctadeca-6,9-diene (C5-Br), replacing 2-aminoethanol (HO-B1-NH₂) with ethylenediamine (NH₂—B1-NH₂), and replacing A1-OH with A10-OH. ¹H NMR (400 MHz, DMSO-d6) δ 8.42 (s, 1H), 7.54 (s, 1H), 7.30 (d, 1H), 7.23 (d, 1H), 7.16 (t, 1H), 6.71 (t, 1H), 5.51 (m, 8H), 4.55 (d, 1H), 4.03 (m, 2H), 3.32 (t, 2H), 2.91 (m, 4H), 2.88 (m, 6H), 2.57 (t, 2H), 2.32-1.69 (m, 17H), 1.36-1.26 (m, 36H), 0.89 (m, 6H).

(23) Compound 28 (A10-B2-C7): Compound 28 with the structural formula

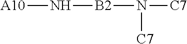

was obtained by replacing (6Z,9Z)-18-bromooctadeca-6,9-diene (C5-Br) with octyl 3-bromopropanoate (C7-Br), replacing 2-aminoethanol (HO-B1-NH₂) with hexamethylenediamine (NH₂—B2-NH₂), and replacing A1-OH with A10-OH. ¹H NMR (400 MHz, DMSO-d6) δ 8.42 (s, 1H), 7.54 (s, 1H), 7.30 (d, 1H), 7.23 (d, 1H), 7.16 (t, 1H), 6.71 (t, 1H), 4.55 (d, 1H), 4.16 (t, 4H), 4.03 (t, 4H), 3.14 (m, 2H), 2.94 (m, 4H), 2.88 (m, 2H), 2.58 (m, 4H), 2.32-1.94 (m, 7H), 1.69-1.27 (m, 34H), 0.89 (m, 6H).

(24) Compound 29 (A11-B2-C9): Compound 29 with the structural formula

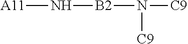

was obtained by replacing (6Z,9Z)-18-bromooctadeca-6,9-diene (C5-Br) with N,N-dioctyl-2-bromo-ethylamine (C9-Br), replacing 2-aminoethanol (HO-B1-NH₂) with hexamethylenediamine (NH₂—B2-NH₂), and replacing A1-OH with A11-OH. ¹H NMR (400 MHz, DMSO-d6) δ 9.70 (s, 1H), 8.03 (t, 2H), 7.58 (d, 1H), 7.39 (d, 1H), 7.12 (d, 2H), 3.71 (s, 1H), 3.11 (t, 24H), 2.45-2.33 (t, 20H), 2.28 (t, 2H), 2.16 (s, 3H), 1.99-1.87 (t, 8H), 1.53-1.26 (m, 112H), 0.92-0.86 (t, 27H).

(25) Compound 30 (A11-B3-C10): Compound 30 with the structural formula

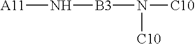

was obtained by replacing (6Z,9Z)-18-bromooctadeca-6,9-diene (C5-Br) with N,N-bis(3-oxodecyl)nonanamide (C10-Br), replacing 2-aminoethanol (HO-B1-NH₂) with 3,4-dithia-1,6-hexanediamine (HO-B3-NH₂), and replacing A1-OH with A11-OH. ¹H NMR (400 MHz, DMSO-d6) δ 9.70 (s, 1H), 8.03 (t, 2H), 7.58 (d, 1H), 7.39 (d, 1H), 7.12 (d, 2H), 3.71 (s, 1H), 3.58 (t, 16H), 3.46 (q, 4H), 3.40 (t, 32H), 3.25 (t, 8H), 2.81-2.64 (t, 12H), 2.48-2.27 (t, 14H), 2.16 (s, 3H), 1.57-1.25 (m, 136H), 0.92-0.88 (t, 27H).

(26) Compound 31 (A11-B3-C11): Compound 31 with the structural formula

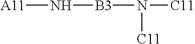

was obtained by replacing (6Z,9Z)-18-bromooctadeca-6,9-diene (C5-Br) with N,N-bis(3-acetyl-nonyl)-propionamide (C11-Br), replacing 2-aminoethanol (HO-B1-NH₂) with 3,4-dithia-1,6-hexanediamine (HO-B3-NH₂), and replacing A1-OH with A11-OH. ¹H NMR (400 MHz, DMSO-d6) δ 9.70 (s, 1H), 8.03 (t, 2H), 7.58 (d, 1H), 7.39 (d, 1H), 7.12 (d, 2H), 4.25 (t, 16H), 3.96 (t, 8H), 3.71 (s, 1H), 3.54 (t, 20H), 2.84-2.28 (t, 40H), 2.16 (s, 3H), 1.63-1.56 (t, 24H), 1.40-1.26 (m, 96H), 0.92-0.88 (t, 27H).

(27) Compound 32 (A12-B3-C11): Compound 32 with the structural formula

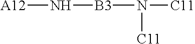

was obtained by replacing (6Z,9Z)-18-bromooctadeca-6,9-diene (C5-Br) with N,N-bis(3-acetyl-nonyl)-propionamide (C11-Br), replacing 2-aminoethanol (HO-B1-NH₂) with 3,4-dithia-1,6-hexanediamine (H₂N-B3-NH₂), and replacing A1-OH with A12-OH. ¹H NMR (400 MHz, DMSO-d6) δ 9.70 (s, 1H), 8.03 (t, 2H), 7.52 (d, 1H), 7.39 (d, 1H), 7.10 (d, 2H), 4.25 (t, 16H), 3.71 (s, 1H), 3.54 (t, 8H), 3.48-3.25 (t, 22H), 2.83-2.64 (t, 8H), 2.44-2.29 (t, 32H), 2.16 (s, 3H), 1.98 (m, 16H), 1.63-1.56 (t, 8H), 1.39-1.26 (m, 96H), 1.20 (s, 9H), 0.92-0.88 (t, 24H).

(28) Compound 33 (A12-B4-C1): Compound 33 with the structural formula

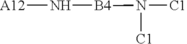

was obtained by replacing (6Z,9Z)-18-bromooctadeca-6,9-diene (C5-Br) with 12-bromododecane (C1-Br), replacing 2-aminoethanol (HO-B1-NH₂) with 4,4-dimethyl-3,5-thioketal-1,7-heptanediamine (H₂N-B4-NH₂), and replacing A1-OH with A12-OH. ¹H NMR (400 MHz, DMSO-d6) δ 9.70 (s, 1H), 8.03 (t, 2H), 7.56 (d, 2H), 7.12 (d, 2H), 3.96 (m, 4H), 3.71 (s, 1H), 3.33 (t, 2H), 3.01 (t, 8H), 2.78-2.51 (t, 12H), 2.31-2.00 (t, 9H), 1.54 (s, 12H), 1.51-1.41 (t, 8H), 1.34-1.27 (m, 80H), 1.20 (s, 9H), 0.88 (t, 12H).

(29) Compound 34 (A12-B4-C2): Compound 34 with the structural formula

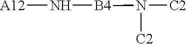

was obtained by replacing (6Z,9Z)-18-bromooctadeca-6,9-diene (C5-Br) with 16-bromohexadecane (C2-Br), replacing 2-aminoethanol (HO-B1-NH₂) with 4,4-dimethyl-3,5-thioketal-1,7-heptanediamine (H₂N-B4-NH₂), and replacing A1-OH with A12-OH. ¹H NMR (400 MHz, DMSO-d6) δ 9.70 (s, 1H), 8.03 (t, 2H), 7.54 (d, 2H), 7.09 (d, 2H), 3.95 (m, 4H), 3.71 (s, 1H), 3.33 (t, 2H), 3.01 (t, 8H), 2.79-2.51 (t, 12H), 2.31-2.00 (t, 9H), 1.54 (s, 12H), 1.51-1.41 (t, 8H), 1.36-1.26 (m, 112H), 1.19 (s, 9H), 0.88 (t, 12H).

### Example 4

According to the reaction steps in Example 2, the following compounds were prepared:

(1) Compound 12 (A4-B2-C6): Compound 12 with the structural formula

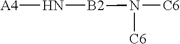

was obtained by replacing 6-aminohexanol (HO-B2-NH₂) with hexamethylenediamine (NH₂—B2-NH₂) and replacing A5-OH with A4-OH. ¹H NMR (400 MHz, DMSO-d6) δ7.73 (s, 1H), 7.61 (d, 1H), 7.51 (d, 1H), 7.44 (t, 1H), 7.16 (t, 1H), 6.75 (t, 1H), 5.37 (s, 2H), 4.38 (t, 1H), 3.45 (m, 2H), 3.22 (t, 2H), 2.68-2.41 (m, 14H), 1.51-1.26 (m, 52H), 1.14 (t, 3H), 0.89 (m, 6H).

(2) Compound 23 (A9-B3-C6): Compound 23 with the structural formula

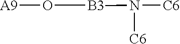

was obtained by replacing 6-aminohexanol (HO-B2-NH₂) with 6-amino-3,4-dithiahexan-1-ol (HO-B3-NH₂), and replacing A5-OH with A9-OH. ¹H NMR (400 MHz, DMSO-d6) δ 9.27 (s, 1H), 7.54 (d, 1H), 7.30 (d, 1H), 7.23 (t, 1H), 7.16 (t, 1H), 4.31 (s, 2H), 4.04 (t, 2H), 3.84 (m, 2H), 3.63 (m, 4H), 2.85 (t, 2H), 2.71-2.30 (m, 16H), 1.74-1.23 (m, 50H), 0.89 (m, 6H).

(3) Compound 27 (A10-B1-C6): Compound 27 with the structural formula

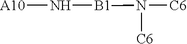

was obtained by replacing 6-amino-3,4-dithiahexan-1-ol (HO-B2-NH₂) with ethylenediamine (NH₂—B1-NH₂) and replacing A5-OH with A10-OH. ¹H NMR (400 MHz, DMSO-d6) δ 8.42 (s, 1H), 7.54 (s, 1H), 7.30 (d, 1H), 7.23 (d, 1H), 7.16 (t, 1H), 6.71 (t, 1H), 5.37 (s, 2H), 4.55 (d, 1H), 3.84 (m, 6H), 3.34 (t, 2H), 2.82-1.69 (m, 15H), 1.38-1.23 (m, 44H), 0.89 (m, 6H).

### Example 5

Compound 1 (A1-B1-C5) was prepared into lipid nanoparticles (LNPs), and then relevant physicochemical properties and in-vitro and in-vivo activities of LNP were tested as follows:

(1) First, lipid nanoparticles (LNPs) were prepared according to the different formulations in Table 1. The specific preparation method was as follows: ionizable lipid, auxiliary lipids (including any one of DOPE, DOPC or DSPC), cholesterol and PEG-DMG were dissolved in an organic solvent according to the corresponding molar ratio and mixed. The mixture was mixed with 6.25 mM sodium acetate buffer (pH=5) containing siRNA (CUU ACG CUG AGU ACU UCG ATT) at a ratio of v/v=3:1 (water:organic solvent) using a microfluidic device. The mixture was transferred to a dialysis bag and dialyzed with PBS buffer solution (pH=7.4) overnight. The mixture was then concentrated using an ultracentrifugal filter. A1-B1-C5 was made into an ionizable lipid and combined with auxiliary lipids, cholesterol and PEG-DMG to form different lipid nanoparticles (LNPs) based on A1-B1-C5 (A1-B1-C5-1 to A1-B1-C5-27). The particle size and PDI (polymer dispersibility index) are shown in Table 2. It can be seen from Table 2 that the LNP prepared under formulation No. 14 has a particle size of <100 nm and good dispersion. The LNP has brain targeting potential in terms of particle size and may be used for subsequent experiments.

Lipid nanoparticles (LNPs) formed from different ionizable lipids with the same formulation were obtained by preparing compounds 1 to 34 prepared in the above examples according to formulation No. 14 in Table 1. The particle size and PDI (polymer dispersibility index) are shown in Table 3. It can be seen from Table 3 that the LNPs prepared from some vincamine compounds have a particle size of <100 nm and good dispersion, which indicates that these LNPs have brain targeting potential in terms of particle size and may be used for subsequent experiments.

TABLE 1
Lipid nanoparticles (LNPs) with different molar ratios

| | Molar ratio | | | | | |
|---|---|---|---|---|---|---|
| Formula | Ionizable | Auxiliary lipids | | | | PEG- |
| No. | lipid | DOPE | DOPC | DSPC | Cholesterol | DMG |
| 1 | 40 | 10 | — | — | 49 | 1 |
| 2 | 40 | 12.5 | — | — | 46 | 1.5 |
| 3 | 40 | 15 | — | — | 43 | 2 |
| 4 | 40 | — | 10 | — | 49 | 1 |
| 5 | 40 | — | 12.5 | — | 46 | 1.5 |
| 6 | 40 | — | 15 | — | 43 | 2 |
| 7 | 40 | — | — | 10 | 49 | 1 |
| 8 | 40 | — | — | 12.5 | 46 | 1.5 |
| 9 | 40 | — | — | 15 | 43 | 2 |
| 10 | 50 | 10 | — | — | 39 | 1 |
| 11 | 50 | 12.5 | — | — | 36 | 1.5 |
| 12 | 50 | 15 | — | — | 33 | 2 |
| 13 | 50 | — | 10 | — | 39 | 1 |
| 14 | 50 | — | 12.5 | — | 36 | 1.5 |
| 15 | 50 | — | 15 | — | 33 | 2 |
| 16 | 50 | — | — | 10 | 39 | 1 |
| 17 | 50 | — | — | 12.5 | 36 | 1.5 |
| 18 | 50 | — | — | 15 | 33 | 2 |
| 19 | 60 | 10 | — | — | 29 | 1 |
| 20 | 60 | 12.5 | — | — | 26 | 1.5 |
| 21 | 60 | 15 | — | — | 23 | 2 |
| 22 | 60 | — | 10 | — | 29 | 1 |
| 23 | 60 | — | 12.5 | — | 26 | 1.5 |
| 24 | 60 | — | 15 | — | 23 | 2 |
| 25 | 60 | — | — | 10 | 29 | 1 |
| 26 | 60 | — | — | 12.5 | 26 | 1.5 |
| 27 | 60 | — | — | 15 | 23 | 2 |

TABLE 2
Particle size and PDI (Polymer Dispersity Index)
of different lipid nanoparticles (LNPs)

| Formulation No. | Size (nm) | PDI |
|---|---|---|
| Compound 1 (A1-B1-C5)-1 | 185.3 | 0.255 |
| Compound 1 (A1-B1-C5)-2 | 132.2 | 0.144 |
| Compound 1 (A1-B1-C5)-3 | >1 μm | — |
| Compound 1 (A1-B1-C5)-4 | 134.1 | 0.218 |
| Compound 1 (A1-B1-C5)-5 | 190.0 | 0.130 |
| Compound 1 (A1-B1-C5)-6 | 158.5 | 0.284 |
| Compound 1 (A1-B1-C5)-7 | 287.5 | 0.076 |
| Compound 1 (A1-B1-C5)-8 | >1 μm | — |
| Compound 1 (A1-B1-C5)-9 | 174.7 | 0.627 |
| Compound 1 (A1-B1-C5)-10 | 102.4 | 0.140 |
| Compound 1 (A1-B1-C5)-11 | 109.3 | 0.181 |
| Compound 1 (A1-B1-C5)-12 | 108.0 | 0.210 |
| Compound 1 (A1-B1-C5)-13 | 216.6 | 0.146 |
| Compound 1 (A1-B1-C5)-14 | 89.3 | 0.213 |
| Compound 1 (A1-B1-C5)-15 | 220.3 | 0.289 |
| Compound 1 (A1-B1-C5)-16 | 100.6 | 0.089 |
| Compound 1 (A1-B1-C5)-17 | 190.0 | 0.197 |
| Compound 1 (A1-B1-C5)-18 | >1 μm | — |
| Compound 1 (A1-B1-C5)-19 | 111.0 | 0.201 |
| Compound 1 (A1-B1-C5)-20 | 140.6 | 0.133 |
| Compound 1 (A1-B1-C5)-21 | 182.0 | 0.184 |
| Compound 1 (A1-B1-C5)-22 | 159.6 | 0.196 |
| Compound 1 (A1-B1-C5)-23 | 214.8 | 0.364 |
| Compound 1 (A1-B1-C5) 5-24 | >1 μm | — |
| Compound 1 (A1-B1-C5)-25 | 198.4 | 0.358 |
| Compound 1 (A1-B1-C5) 5-26 | 218.4 | 0.05 |
| Compound 1 (A1-B1-C5)-27 | 172.8 | 0.199 |

TABLE 3
Particle size and PDI (Polymer Dispersity Index)
of lipid nanoparticles (LNPs) formed from different
ionizable lipids in the same formulation

| Formulation No. | Size (nm) | PDI |
|---|---|---|
| Compound 1 (A1-B1-C5)-14 | 92.6 | 0.225 |
| Compound 2 (A5-B2-C6)-14 | 89.3 | 0.213 |
| Compound 3 (A1-B1-C2)-14 | 114.6 | 0.243 |
| Compound 4 (A1-B1-C3)-14 | 171.3 | 0.136 |
| Compound 5 (A1-B2-C4)-14 | 164.2 | 0.228 |
| Compound 6 (A2-B2-C8)-14 | 198.5 | 0.189 |
| Compound 7 (A2-B3-C9)-14 | >1 μm | — |
| Compound 8 (A2-B3-C10)-14 | 90.3 | 0.115 |
| Compound 9 (A3-B4-C3)-14 | 273.6 | 0.212 |
| Compound 10 (A3-B1-C4)-14 | 165.4 | 0.315 |
| Compound 11 (A3-B1-C5)-14 | >1 μm | — |
| Compound 12 (A4-B2-C6)-14 | 167.7 | 0.305 |
| Compound 13 (A4-B2-C7)-14 | 112.9 | 0.214 |
| Compound 14 (A5-B3-C8)-14 | 100.3 | 0.127 |
| Compound 15 (A5-B3-C9)-14 | 126.9 | 0.411 |
| Compound 16 (A6-B4-C10)-14 | >1 μm | — |
| Compound 17 (A6-B4-C11)-14 | 93.2 | 0.165 |
| Compound 18 (A7-B1-C1)-14 | 115.9 | 0.247 |
| Compound 19 (A7-B1-C2)-14 | 108.4 | 0.175 |
| Compound 20 (A8-B2-C3)-14 | 91.3 | 0.198 |
| Compound 21 (A8-B2-C4)-14 | 88.5 | 0.166 |
| Compound 22 (A8-B3-C5)-14 | >1 μm | — |
| Compound 23 (A9-B3-C6)-14 | 86.6 | 0.201 |
| Compound 24 (A9-B4-C7)-14 | 105.6 | 0.141 |
| Compound 25 (A9-B4-C8)-14 | 143.6 | 0.315 |
| Compound 26 (A10-B1-C5)-14 | 150.4 | 0.251 |
| Compound 27 (A10-B1-C6)-14 | >1 μm | — |
| Compound 28 (A10-B2-C7)-14 | 116.1 | 0.196 |
| Compound 29 (A11-B2-C9)-14 | 104.7 | 0.170 |
| Compound 30 (A11-B3-C10)-14 | 98.3 | 0.157 |
| Compound 31 (A11-B3-C11)-14 | 110.3 | 0.249 |
| Compound 32 (A12-B3-C11)-14 | 137.6 | 0.334 |
| Compound 33 (A12-B4-C1)-14 | 179.3 | 0.225 |
| Compound 34 (A12-B4-C2)-14 | 158.4 | 0.114 |

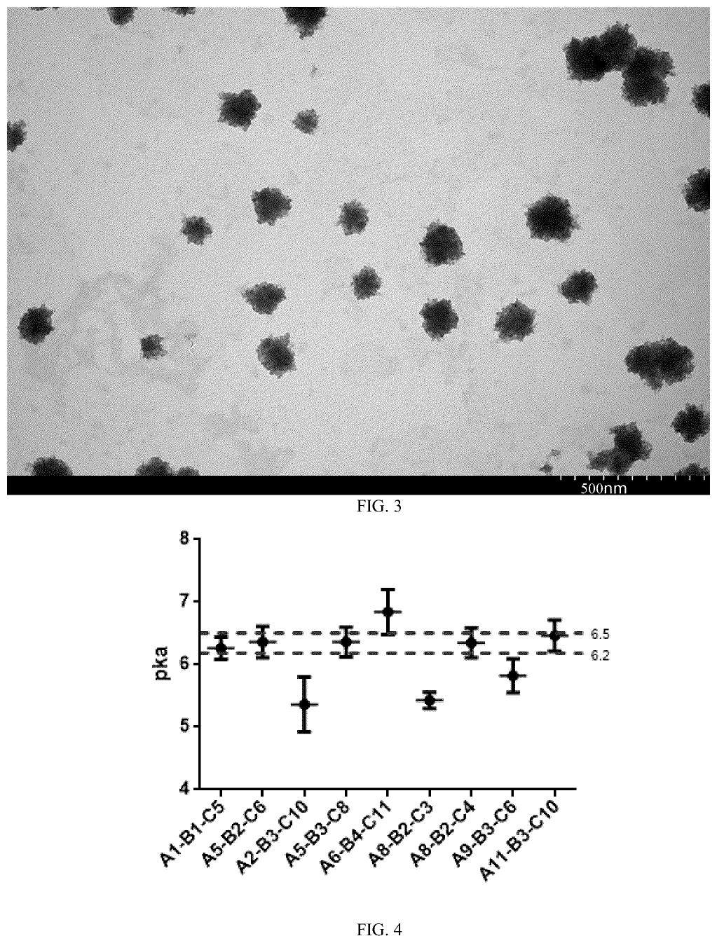

(2) The lipid nanoparticles (LNPs) prepared using compound 2 (A5-B2-C6) as the ionizable lipid according to formulation 14 in Table 1 were analyzed by transmission electron microscopy. The results are shown in FIG. 3. As shown in FIG. 3, the LNPs have a particle size of about 100 nm.

(3) Lipid nanoparticles (A1-B1-C5-14) were prepared using A1-B1-C5 as an ionizable lipid using formulation 14 in Table 1. Lipid nanoparticles (A5-B2-C6-14) were prepared by using A5-B2-C6 as an ionizable lipid according to the formulation 14 in Table 1. Lipid nanoparticles (A2-B3-C10-14) were prepared by using A2-B3-C10 as an ionizable lipid according to the formulation 14 in Table 1. Lipid nanoparticles (A5-B3-C8-14) were prepared by using A5-B3-C8 as an ionizable lipid according to the formulation 14 in Table 1. Lipid nanoparticles (A6-B4-C11-14) were prepared by using A6-B4-C11 as an ionizable lipid according to the formulation 14 in Table 1. Lipid nanoparticles (A8-B2-C3-14) were prepared using A8-B2-C3 as the ionizable lipid according to the formulation 14 in Table 1. Lipid nanoparticles (A8-B2-C4-14) were prepared using A8-B2-C4 as the ionizable lipid according to the formulation 14 in Table 1. Lipid nanoparticles (A9-B3-C6-14) were prepared using A9-B3-C6 as the ionizable lipid according to the formulation 14 in Table 1. Lipid nanoparticles (A11-B3-C10-14) were prepared using A11-B3-C10 as the ionizable lipid using formulation 14 in Table 1. The pKa of the lipid nanoparticles prepared from the vincamine derivatives was determined using the TNS fluorescence assay. Specifically, the prepared lipid nanoparticles were diluted to 2 mM in PBS buffer. A buffer containing 130 mM of NaCl, 10 mM of ammonium acetate, 10 mM of Hepes, and 10 mM of MES was prepared, and the lipid nanoparticles were diluted to 100 μM. The buffer was divided into eight groups, and the pH of the groups was adjusted to 3, 4, 5, 6, 7, 8, 9, and 10 using NaOH or HCl solutions. The anionic fluorescent dye 2-(p-toluidino)-6-naphthalenesulfonic acid (TNS) was dissolved in distilled water to form a solution with a concentration of 100 M, and the final concentration of the lipid nanoparticles was adjusted to 1 μM. The fluorescence intensity of each group was measured using a multifunctional microplate reader at room temperature with an excitation wavelength of 321 nm and an emission wavelength of 445 nm. The pKa was defined as the pH at which the fluorescence intensity reached half of the maximum. The final results are shown in FIG. 4. It may be seen from FIG. 4 that the pKa value of the LNPs prepared from the vincamine derivative of the present application is between 6.2 and 6.5, which is conducive to the efficient encapsulation and lysosomal escape of nucleic acids.

(4) The encapsulation of siRNA by lipid nanoparticles formed from different vincamine derivatives in Table 2 was tested as follows:

The concentration of siRNA in lipid nanoparticles formed from different vincamine derivatives in Table 2 was quantified using the Quant-iT RiboGreen RNA Assay kit. The method was as follows: the dialyzed lipid nanoparticles were diluted in Tris-EDTA (TE) buffer in a 96-well plate. Meanwhile, the dialyzed lipid nanoparticles were diluted into TE buffer containing 2% Triton X-100 for 30 min. Then, RiboGreen working solution was added to the corresponding wells. The fluorescence intensity at an excitation wavelength of 485 nm and an emission wavelength of 530 nm was measured using a microplate reader to reflect the amount of free siRNA and dialyzed siRNA. The siRNA concentration was calculated using the siRNA standard curve, and the encapsulation efficiency was calculated. The results are shown in Table 4. It may be seen from Table 4 that the LNPs prepared from the vincamine derivative of the present application has a good encapsulation efficiency for nucleic acids, which is higher than 90%.

TABLE 4
Encapsulation efficiency of siRNA in lipid nanoparticles
prepared with different vincamine derivatives

| Formulation No. | Encapsulation efficiency (%) |
|---|---|
| Compound 1 (A1-B1-C5)-14 | 90.1 |
| Compound 2 (A5-B2-C6)-14 | 87.6 |
| Compound 6 (A2-B2-C8)-14 | 75.3 |
| Compound 9 (A3-B4-C3)-14 | 62.4 |
| Compound 12 (A4-B2-C6)-14 | 83.8 |
| Compound 16 (A6-B4-C10)-14 | 91.4 |
| Compound 19 (A7-B1-C2)-14 | 78.6 |
| Compound 22 (A8-B3-C5)-14 | 83.7 |
| Compound 24 (A9-B4-C7)-14 | 93.5 |
| Compound 26 (A10-B1-C5)-14 | 80.4 |
| Compound 30 (A11-B3-C10)-14 | 95.9 |
| Compound 32 (A12-B3-C11)-14 | 62.1 |

(5) The in vitro transfection efficiency of siRNA-encapsulated lipid nanoparticles prepared from vincamine derivatives was imaged using a fluorescence microscope:

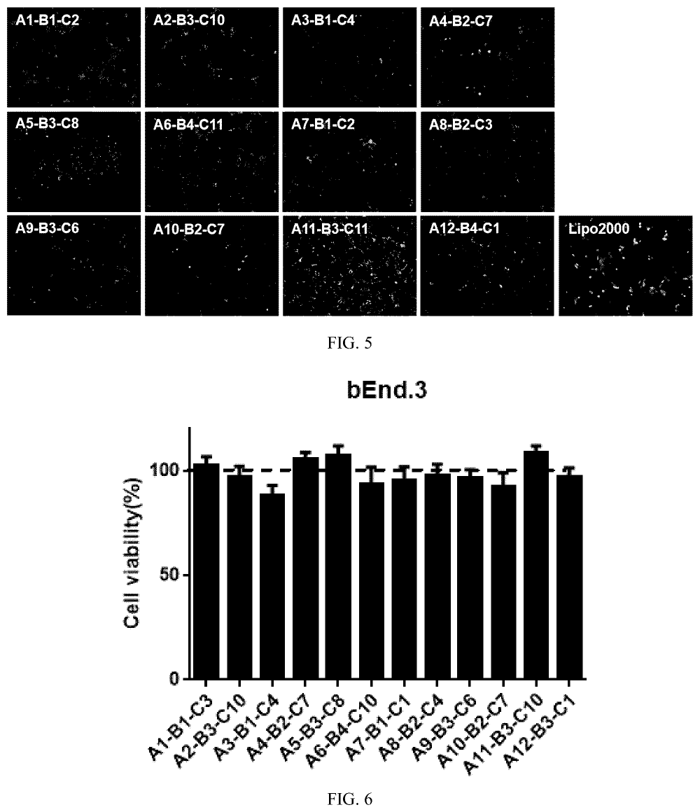

The bEnd.3 cells were seeded into 6-well plates (30% confluency) and cultured in 1 mL of complete medium without antibodies for 12 hours. The culture medium was then replaced with serum-free and antibiotic-free medium. Lipid nanoparticles encapsulated with FAM fluorescent-tagged siRNA (compound 3 (A1-B1-C2), compound 8 (A2-B3-C10), compound 10 (A3-B1-C4), compound 13 (A4-B2-C7), compound 14 (A5-B3-C8), compound 17 (A6-B4-C11), compound 19 (A7-B1-C2), compound 20 (A8-B2-C3), compound 23 (A9-B3-C6), compound 28 (A10-B2-C7), compound 31 (A11-B3-C11), compound 33 (A12-B4-C1), and Lipo2000 (control group) as examples) were added to the cells, and the cells were incubated at 37° C. in a 5% CO₂ environment for 6 hours. The culture medium was replaced with complete culture medium free of double-antibody, and the culture was continued for 18 hours. The FAM fluorescence intensity of the different administration groups was observed under a fluorescence microscope. The results are shown in FIG. 5. It may be seen from FIG. 5 that, compared with the gold-standard transfection reagent Lipofectamine 2000, LNPs prepared from some vincamine compounds can better carry siRNA for transfection into cells.

(6) The cytotoxicity of lipid nanoparticles formed from different vincamine derivatives in Table 2 was determined using MTT:

The bEnd.3 cells were seeded in 96-well plates (6000 cells per well) and cultured in 100 μL of culture medium for 12 hours. Then, lipid nanoparticles formed from different vincamine derivatives listed in Table 2 (compound 3 (A1-B1-C2), compound 8 (A2-B3-C10), compound 10 (A3-B1-C4), compound 13 (A4-B2-C7), compound 14 (A5-B3-C8), compound 17 (A6-B4-C11), compound 19 (A7-B1-C2), compound 20 (A8-B2-C3), compound 23 (A9-B3-C6), compound 28 (A10-B2-C7), compound 31 (A11-B3-C11), and compound 33 (A12-B4-C1)) were added to the cells, and the cells were incubated at 37° C. in a 5% CO₂ atmosphere for 48 hours. At the end of the experiment, 3-(4,5)-dimethylthiazolylazol-2-yl)-2,5-diphenyltetrazolium bromide (MTT) solution (5 mg/ml, 20 L) was added, and the cells were further incubated at 37° C. for 4 hours. The cell viability was calculated by measuring the absorbance of each well at 570 nm using a microplate reader, and the results are shown in FIG. 6. It may be seen from FIG. 6 that, the LNPs prepared from the vincamine derivative of the present application has low cytotoxicity and high biocompatibility.

(7) The effects of lipid nanoparticles formed from different vincamine derivatives in Table 2 on cerebral microvascular blood flow in mice were assessed by laser speckle contrast imaging:

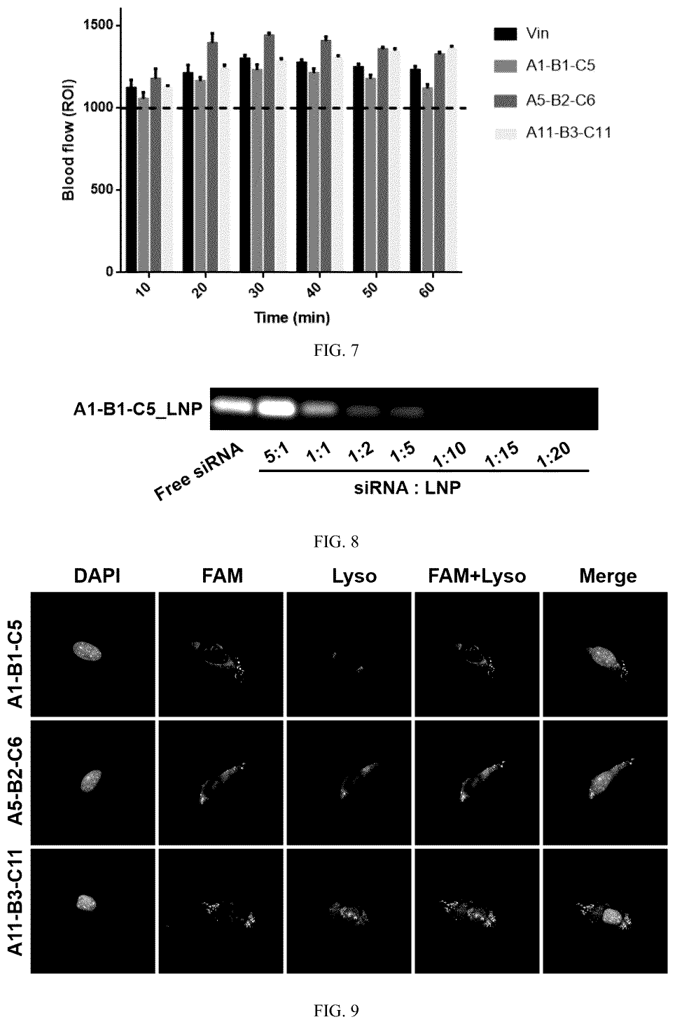

The mice were anesthetized with isoflurane and fixed in a stereotaxic apparatus. The skull skin was incised along the midline, and the cerebral microvascular blood flow of the mice was detected using a laser speckle imager. After the mice were stable for 30 min, 200 L of lipid nanoparticles formed from different vincamine derivatives listed in Table 2 were administered via the tail vein at a dose of 30 mg/kg. Laser speckle blood flow images were recorded within 60 min after administration to identify the cerebral microvascular blood flow perfusion (within ROIs). The results are shown in FIG. 7. It may be seen from FIG. 7 that, LNPs prepared from some vincamine compounds have the effect of increasing cerebral microvascular blood flow in mice.

(8) Taking the lipid nanoparticles containing siRNA formed from the vincamine derivative (A1-B1-C5) in Table 2 as an example, the N/P ratio was determined by a gel electrophoresis:

siRNA (1OD) was dissolved in 125 μL of DEPC water. Lipid nanoparticles (LNPs) formed from vincamine derivative compound 1 (A1-B1-C5) in Table 2 and siRNA solution were mixed at siRNA/LNP mass ratios of 5:1, 1:1, 1:2, 10:1, 1:5, 1:10, 1:15, and 1:20. The mixture was incubated at room temperature for 30 min. The siRNA binding ability of the LNPs was investigated by agarose gel electrophoresis. The LNPs were mixed with 6× loading buffer and loaded onto a 2% agarose gel containing 0.01% gel stain (GlodView). Electrophoresis was performed in 1×TAE buffer at 90 V for 30 min. The results were recorded using a gel image analysis system (Tanon, China) at a UV wavelength of 320 nm, and the results are shown in FIG. 8. It may be seen from FIG. 8 that, the LNPs prepared from the vincamine derivative of the present application may completely encapsulate siRNA when the siRNA/LNP mass ratio is greater than 1:10.

(9) The endosomal escape effects of siRNA-encapsulated lipid nanoparticles formed from different vincamine derivatives in Table 2 (compound 1 (A1-B1-C5), compound 2 (A5-B2-C6), and compound 31 (A11-B3-C11) as examples) were imaged using a confocal microscope:

bEnd.3 cells were seeded in 12-well plates (20000 cells per well) and cultured in 500 μL of culture medium for 12 hours. The culture medium was discarded and replaced with fresh complete culture medium. LNP@FAM-siRNA (lipid nanoparticles formed from different vincamine derivatives as listed in Table 2) was added to the cells and incubated at 37° C. in a 5% CO₂ environment in the dark for 2, 4, 6, and 8 hours. After uptake, the culture medium containing LNPs (lipid nanoparticles formed from different vincamine derivatives as listed in Table 2 (compound 1 (A1-B1-C5), compound 2 (A5-B2-C6), and compound 31 (A11-B3-C11) as examples)) was discarded, the cells were washed three times with PBS buffer, and incubated with Lysotracker Red (1 μM) at 37° C. in the dark for 2 hours. After incubation, the supernatant was discarded, and the cells were washed three times with PBS buffer. The cells were fixed with 4% paraformaldehyde at room temperature for 30 min. The fixative was discarded and the cells were rinsed three times with PBS buffer. Hoechst 33342 nuclear stain was added, and the cells were incubated in the dark for 10 min. After three rinses with PBS buffer, fluorescence colocalization images were taken using a high-content imaging system in confocal mode. The results are shown in FIG. 9. It may be seen from FIG. 9 that, the LNPs prepared from the vincamine derivative of the present application may carry siRNA and efficiently escape from lysosomes.

(10) The effect of lipid nanoparticles formed from the vincamine derivative (compound 2 (A5-B2-C6) as an example) on cellular ROS levels was detected using a single cell analyzer:

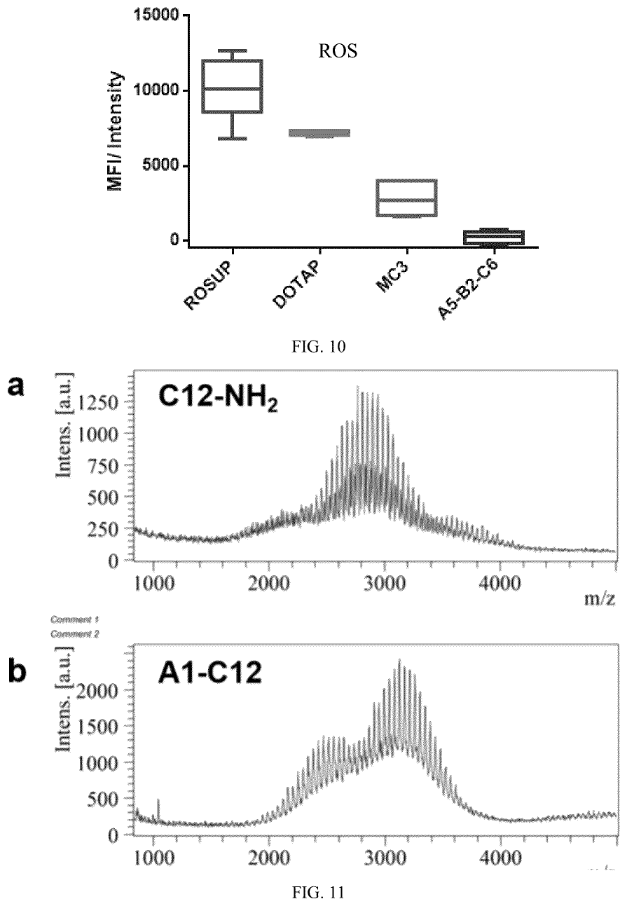

To detect cellular oxidative stress and intracellular reactive oxygen species (ROS) levels, a dichlorodihydrofluorescein diacetate (DCFH-DA) reactive oxygen species assay kit was used to detect the green fluorescence signal of ROS. bEnd.3 cells were seeded in cell culture dishes (50000 cells per dish) and cultured in 1 mL of culture medium for 12 hours. The culture medium in the wells was discarded and replaced with fresh complete medium. Lipid nanoparticles formed from the vincamine derivative (compound 2 (A5-B2-C6)) were added to the cells, and the cells were incubated in the dark at 37° C. in a 5% CO₂ atmosphere for 2 hours. The cells in each group were incubated in PBS buffer containing 10 M DCFH-DA at 37° C. for 30 min. The cells were then washed three times in PBS buffer solution containing 0.1% BSA and fixed on glass slides. ROS levels were detected at the single-cell level using a fiber optic probe with a tip of approximately 10 m. The tip was inserted into the cells fixed in PBS, and photon counts were continuously detected and calculated within 150 s. The results are shown in FIG. 10. It may be seen from FIG. 10 that, compared with the positive control of the kit and the commercially available cationic lipid DOTAP and ionizable lipid Dlin-MC3-DMA (MC3), which induce cells to produce high ROS levels and lead to cytotoxicity, the LNPs prepared by the vincamine compound have low cytotoxicity and high biocompatibility.

### Example 6

A vincamine derivative (compound 35 (A1-C12)) was prepared, specifically including the following steps:

(1) NH₂-PEG₂₀₀₀-DSPE (60 mg, 0.02 mmol) (whose mass spectrum is shown in FIG. 11a), A1 (13 mg, 0.04 mmol), and benzotriazol-1-yloxy-tris(pyrrolidino)phosphonium hexafluorophosphate (PyBOP, 31.2 mg, 0.06 mmol) were dissolved in 3 mL of N,N-dimethylformamide, and the reaction mixture was stirred at room temperature for 24 hours.

(2) The reaction solution was placed in a dialysis bag and dialyzed with pure water for 24 hours. The aqueous solution in the dialysis bag was taken out and freeze-dried to obtain a vincamine derivative having the structural formula A1-PEG₂₀₀₀-DSPE, i.e. compound 35 (A1-C12) (whose mass spectrum is shown in FIG. 11b).

### Example 7

1. A vincamine derivative (compound 36 (A1-C13)) was prepared, specifically including the following steps:

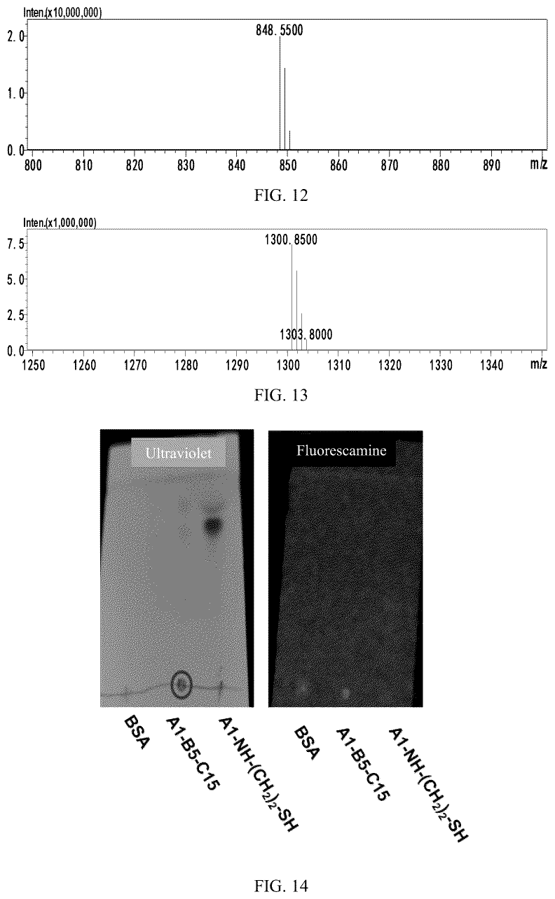

(1) Compound C13-NH₂ (Doxorubicin, DOX-NH₂) (20 mg, 0.037 mmol) and compound A1-OH (16.8 mg, 0.052 mmol) were dissolved in 2 mL of N,N-dimethylformamide and stirred. Benzotriazol-1-yl-oxy-tris(pyrrolidino)phosphonium hexafluorophosphate (27 mg, 0.052 mmol) was added, and the pH was adjusted to 7-8 with N,N-diisopropylethylamine. The reaction solution was reacted at room temperature for 1 hour. N,N-dimethylformamide was then removed by rotary evaporation, and the mixture was dissolved in ethyl acetate (20 mL), washed with 0.10 M hydrochloric acid solution, and then washed to neutral with saturated sodium chloride solution. After drying over anhydrous sodium sulfate. The mixture was filtered under reduced pressure and the organic phase was concentrated by rotary evaporation, and further purified by trituration with methyl tert-butyl ether to obtain a vincamine derivative with the structural formula A1-DOX, i.e., compound 36 (A1-C13). (¹H NMR (400 MHz, DMSO-d6) δ 12.04 (s, 2H), 8.32 (s, 1H), 7.90-7.40 (m, 6H), 6.90 (t, 1H), 6.70 (d, 1H), 5.37 (s, 1H), 4.90 (d, 2H), 4.70-4.65 (m, 4H), 4.22 (t, 1H), 3.90 (s, 3H), 3.65 (m, 3H), 3.37 (d, 2H), 2.65-1.42 (m, 16H), 1.11 (d, 3H), 0.99 (m, 3H). The mass spectrum is shown in FIG. 12).

2. A vincamine derivative (compound 37 (A1-B5-C14)) was prepared, specifically including the following steps:

(1) Compound A1-OH (60 mg, 0.17 mmol) and compound H₂N-B1-NH₂ (44.7 mg, 0.27 mmol) were dissolved in 2 mL of N,N-dimethylformamide, and the mixture was stirred. Benzotriazol-1-yloxy-tris(pyrrolidino)phosphonium hexafluorophosphate (147 mg, 0.28 mmol) was added, and the mixture was reacted at room temperature overnight. After concentration under reduced pressure, the mixture was dissolved in dichloromethane (5 mL), extracted with 10% anhydrous citric acid and saturated brine in sequence, dried over anhydrous sodium sulfate, and concentrated under reduced pressure to remove the organic phase. After the mixture was dissolved in a small amount of methanol (0.5 mL), the mixture was washed with methyl tert-butyl ether (15 mL) three times. After the mixture was dissolved in dichloromethane (1.6 mL), trifluoroacetic acid (0.4 mL) was added and the mixture was stirred at room temperature for 1 hour. 30 mL of methyl tert-butyl ether was added for precipitation, and the precipitate was dried to obtain A1-B5-NH₂.

(2) Paclitaxel (PTX) (25.1 mg, 0.029 mmol) and succinic anhydride (37.5 mg, 0.37 mmol) were added to 0.6 mL of pyridine, and the mixture was stirred at room temperature for 3 hours. The solvent was removed in vacuo and the residue was washed with 1 mL of water and dried. The precipitate was then dissolved in acetone, ice water was added dropwise to the solution to induce crystallization, and PTX-COOH was obtained after drying;

(3) PTX-COOH (30 mg, 0.031 mmol) and A1-B5-NH₂ (16.8 mg, 0.052 mmol) were dissolved in 2 mL of N,N-dimethylformamide and stirred. Benzotriazol-1-yl-oxy-tris(pyrrolidino)phosphonium hexafluorophosphate (24.6 mg, 0.047 mmol) was added, and the pH was adjusted to 7-8 with N,N-diisopropylethylamine. The mixture was reacted at room temperature for 2 hours. After concentration under reduced pressure to remove N,N-dimethylformamide, the mixture was dissolved in dichloromethane (5 mL), extracted three times with 10% anhydrous citric acid (4 mL) and saturated brine, dried over anhydrous sodium sulfate, and concentrated by rotary evaporated to obtain a vincamine derivative with the structural formula A1-B5-PTX, namely compound 37 (A1-B5-C14). (¹H NMR (400 MHz, DMSO-d6) δ 8.87 (s, 1H), 8.41 (s, 1H), 8.05-7.89 (m, 5H), 7.68-7.27 (m, 15H), 6.77 (s, 1H), 6.70 (s, 1H), 6.50 (d, 1H), 6.21 (s, 1H), 6.10 (d, 1H), 5.13-5.03 (m, 4H), 4.27 (d, 1H), 3.73-3.66 (m, 4H), 3.55 (s, 1H), 3.42 (m, 2H), 2.68-2.41 (m, 11H), 2.19 (s, 6H), 2.11-1.25 (m, 16H), 1.01 (s, 6H), 0.89 (s, 3H), the mass spectrum is shown in FIG. 13).

### Example 8

A vincamine derivative (compound 38 (A1-B5-C15)) was prepared, specifically including the following steps:

(1) Compound A1-OH (50 mg, 0.15 mmol), cystamine dihydrochloride (52 mg, 0.23 mmol), and benzotriazol-1-yl-oxy-tris(pyrrolidino)phosphonium hexafluorophosphate (PyBOP, 120 mg, 0.23 mmol) were dissolved in 6 mL of N,N-dimethylformamide/methanol (5/1, v/v). 50 μL of triethylamine was added dropwise. The reaction mixture was stirred at room temperature for 2 hours and then added dropwise to 30 mL of methyl tert-butyl ether (a large amount of yellow semisolid precipitate formed). The precipitate was washed twice with methyl tert-butyl ether and dried under vacuum to obtain the crude product. The crude product was dissolved in 3 mL of N,N-dimethylformamide, and tris(2-carboxyethyl)phosphine hydrochloride (50 mg, 0.17 mmol) was dissolved in 200 L of water and added dropwise to this solution. The reaction mixture was stirred at room temperature for 2 hours, concentrated under reduced pressure, and precipitated by the addition of water. The precipitate was dried to obtain an intermediate with the structure A1-NH—(CH₂)₂—SH.

(2) The intermediate with the structure of A1-NH—(CH₂)₂—SH (7.8 mg, 0.02 mmol) and NHS-PEG₈-Mal (6.9 mg, 0.01 mmol) were dissolved in 100 L N,N-dimethylformamide and stirred at room temperature for 1 hour. The reaction mixture was added dropwise into a solution of BSA in PBS (68 mg, 0.001 mmol) and stirred at room temperature for 3 hours. The reaction solution was placed in an 8000-14000 Da dialysis bag and dialyzed with pure water for 24 hours. Finally, the aqueous solution in the dialysis bag was taken out and freeze-dried to obtain the vincamine derivative, compound 38, with the structural formula A1-B5-C15.

(3) The product was verified by TLC (as shown in FIG. 14) was performed using chloroform and methanol in a volume ratio of 3:1 as the developing solvent, and UV and fluorescamine were used for color development. Compared with the BSA spot, the compound 38 (A1-B5-C15) spot showed a UV signal, indicating that the coupling was successful.

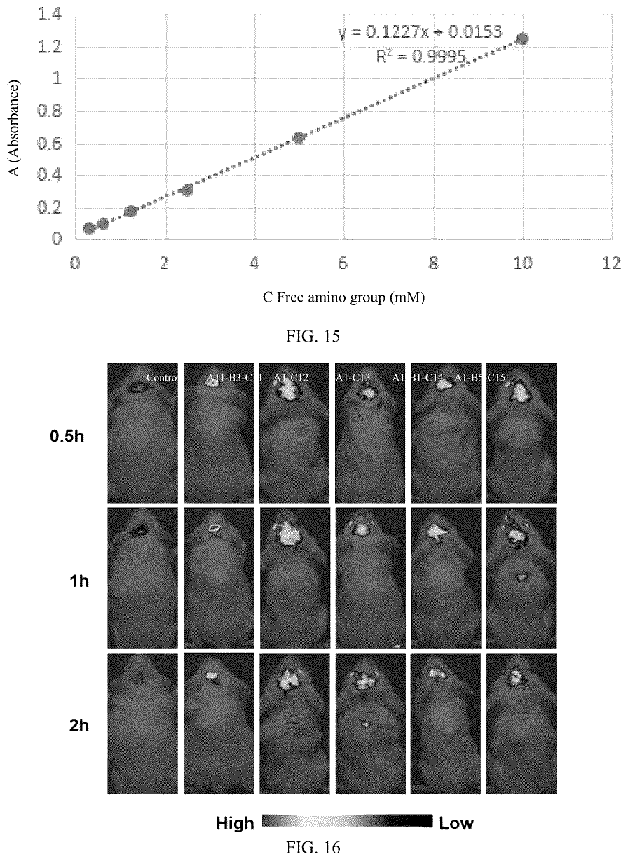

(4) The free amino group content of BSA and A1-B9-C15 was determined using OPA. 40 mg of OPA was dissolved in 1 mL of methanol, and then 25 mL of 0.1 M sodium borate, 200 mg of DMA and 5 mL of 10% SDS were added, and the volume was adjusted to 50 mL with water to obtain the OPA reagent. 65 μL of sample (1% protein) was mixed with 3 mL of the OPA reagent solution. After 2 min, the absorbance was measured at 340 nm using an ultraviolet spectrophotometer. This absorbance corresponds to the alkylisoindole derivative formed after the reaction of OPA with the free amino group. A standard curve was obtained by measuring the absorbance after the reaction of L-leucine (0-10 mM) with the OPA reagent (as shown in FIG. 15). The free amino group content of the protein sample was calculated based on the standard curve (as shown in Table 5), and the coupling efficiency with the structure of A1-NH—(CH₂)₂—SH was estimated. Compared with BSA, the number of free amino groups in A1-B5-C15 decreased by about 10, indicating that on average, approximately 11 small molecules were coupled to each BSA.

TABLE 5
Number of free amino groups in A1-B5-BSA

| | BSA | A1-B5-C15 |
|---|---|---|
| A (Absorbance) | 0.713 | 0.516 |
| Number of free amino | 38 | 27 |
| groups | | |
| Number of modified | 0 | 11 |
| small molecules | | |

### Example 9

The in vivo brain targeting of vincamine derivatives (compound 31 (A11-B3-C11), compound 35 (A1-C12), compound 36 (A1-C13), compound 37 (A1-B1-C14), and compound 38 (A1-B5-C15) as examples) was imaged using in vivo imaging:

Preparations loaded with DiD or compounds labeled with Cy5 (compound 31 (A11-B3-C11), compound 35 (A1-C12), compound 36 (A1-C13), compound 37 (A1-B1-C14), and compound 38 (A1-B5-C15)) were injected into mice via the tail vein. Fluorescence images were collected at predetermined time points (0.5, 1, and 2 hours) using the VISQUE in vivo Smart-LF System. The results are shown in FIG. 16. As can be seen from FIG. 16 that, compared with the control group (Control), the vincamine derivatives of the present application has brain targeting properties.

In summary, the present application discloses a vincamine derivative and a preparation method therefor. The vincamine derivative has the following advantages: (i) Modification of the tail chain increases the lipid solubility of vincamine compounds without affecting the cerebral blood flow regulation of vincamine itself, thereby helping the carried drug cross the blood-brain barrier, exert a brain-protective effect, and improve cerebral microcirculatory disorders. (ii) The tertiary amine group in the parent nucleus structure of the vincamine derivative is ionizable under acidic conditions, which enables efficient delivery and lysosomal escape of nucleic acid drugs through charge adsorption, thereby improving intracellular transport. (iii) The vincamine derivative inherits various pharmacological activities inherent in vincamine, and has a high safety profile. Therefore, the vincamine derivative disclosed in the present application has good application prospects in brain-targeted delivery of drugs for treating brain diseases.

Finally, it should be noted that the foregoing embodiments are merely intended for illustrating the technical solutions of the present application and do not limit the present application. Although the present application is described in detail with reference to the preferred embodiments, those of ordinary skill in the art understand that the technical solutions of the present application can be modified or equivalently substituted without departing from the essence and scope of the technical solutions of the present application and should all be covered by the scope of the claims of the present application.

What is claimed is:

1. A vincamine derivative, wherein the vincamine derivative has a structural formula of any one of 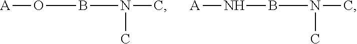
wherein A is a group formed after vincamine and a derivative thereof having an indole-fused ring system lose a hydroxyl group;
B is any one of —(CH₂)ₙ—, —(CH₂)ₙ—S—S—(CH₂)ₙ—, —(CH₂)ₙ-TK-(CH₂)ₙ— or —(CH₂)ₙ—S-Mal-(PEG)ₘ-NHS—, wherein n and m are positive integers; and
C is any one of a linear alkyl group having 10 to 18 carbon atoms, a linear alkenyl group having 10 to 18 carbon atoms, a hydroxyl-substituted linear alkyl group having 10 to 18 carbon atoms, a group with a chemical formula CₓH₂ₓ₊₁OCO(CH₂)_{y}—, a group with a chemical formula (CₐH₂ₐ₊₁)₂N(CH₂)_{b}—, a group with a chemical formula (C_{d}H_{2d+1}OCH₂CH₂)₂NCO(CH₂)ₑ—, a group with a chemical formula (C_{d}H_{2d+1}COOCH₂CH₂)₂NCO(CH₂)ₑ—, DSPE-PEG-, PEG-, a group formed after Doxorubicin loses an amino group, a group formed after paclitaxel is carboxylated at a 2′-OH site, or an amino acid chain, wherein x, a, b, d, and e are all positive integers.

2. The vincamine derivative according to claim 1, wherein A is any one of structural formulas A1 to A12:

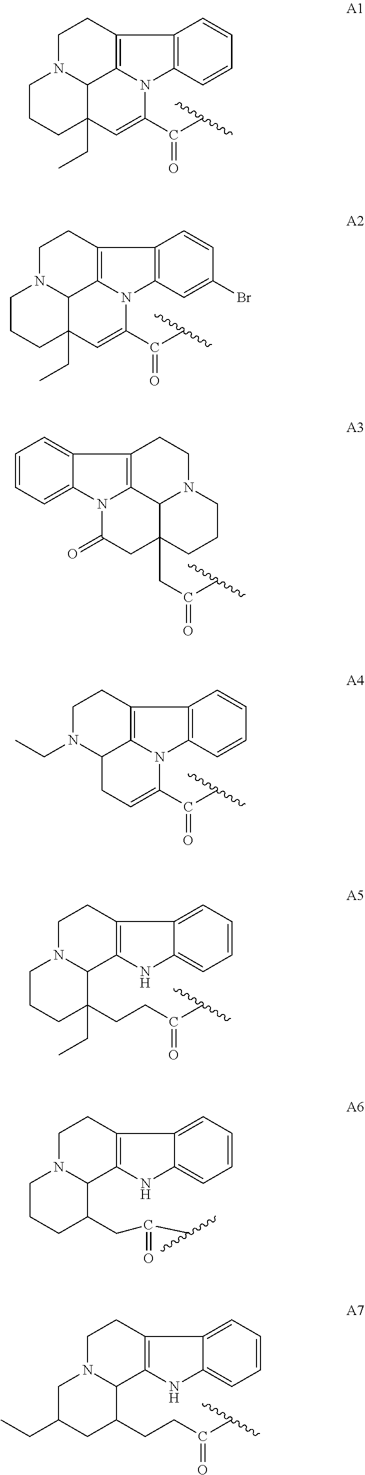

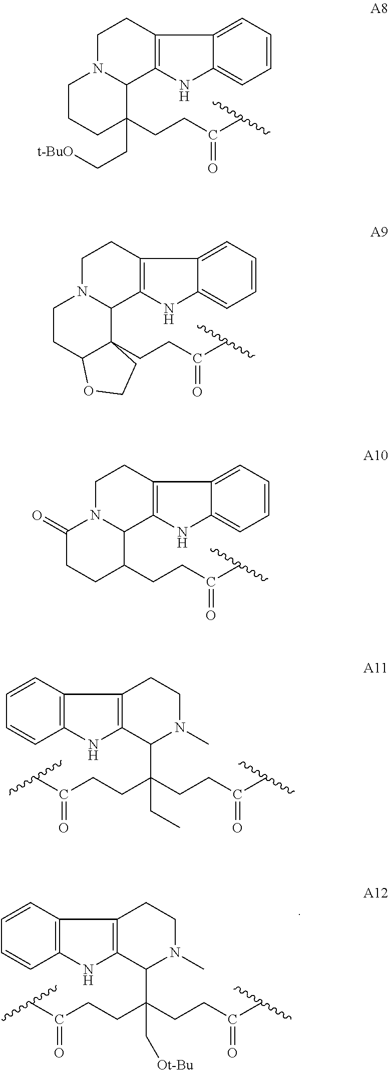 [SMILES: *C(=O)CCC1(CCOC(C)(C)C)CCCN2CCc3c([nH]c4ccccc34)C21 ; *C(=O)CCC12CCOC1CCN1CCc3c([nH]c4ccccc34)C12 ; *C(=O)CCC1CCC(=O)N2CCc3c([nH]c4ccccc34)C12 ; *C(=O)CCC(CC)(CCC(*)=O)C1c2[nH]c3ccccc3c2CCN1C ; *C(=O)CCC(CCC(*)=O)(COC(C)(C)C)C1c2[nH]c3ccccc3c2CCN1C]

3. The vincamine derivative according to claim 1, wherein B is any one of B1 to B5, B1 is —(CH₂)₂—, B2 is —(CH₂)₆—, B3 is —(CH₂)₂—S—S—(CH₂)₂—, B4 is —(CH₂)₂—S—C(CH₃)₂—S—(CH₂)₂—, and B5 is —(CH₂)₂—S-Mal-PEG-NHS—.

4. The vincamine derivative according to claim 1, wherein C or C′ is any one of structural formulas C1 to C14: 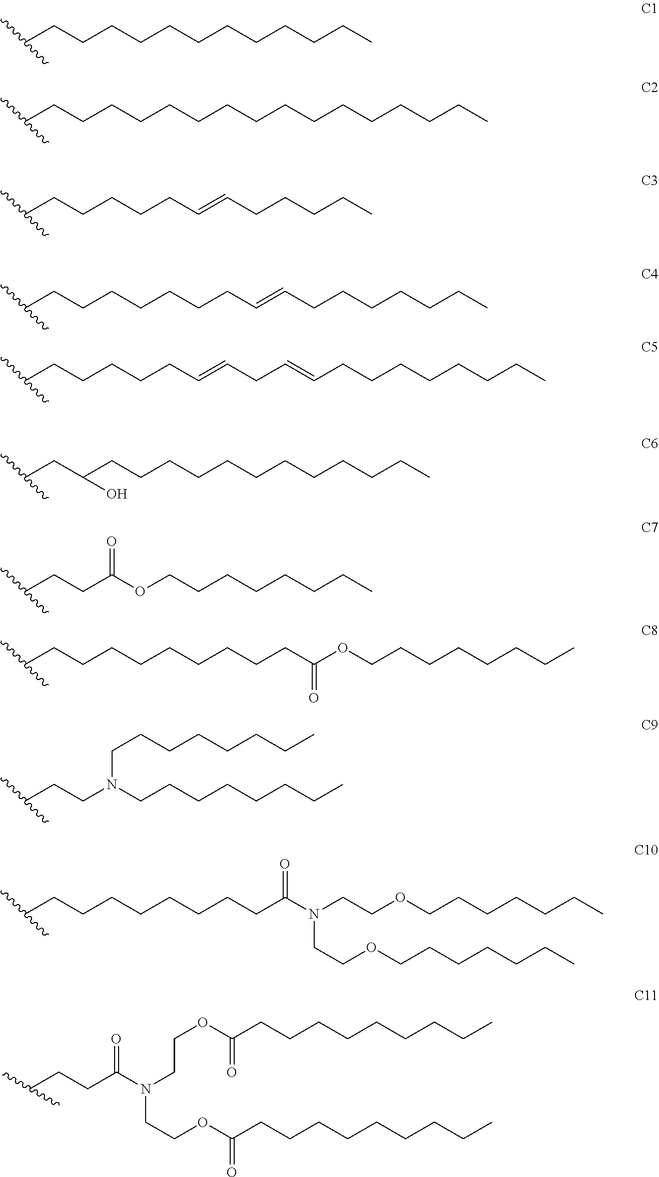 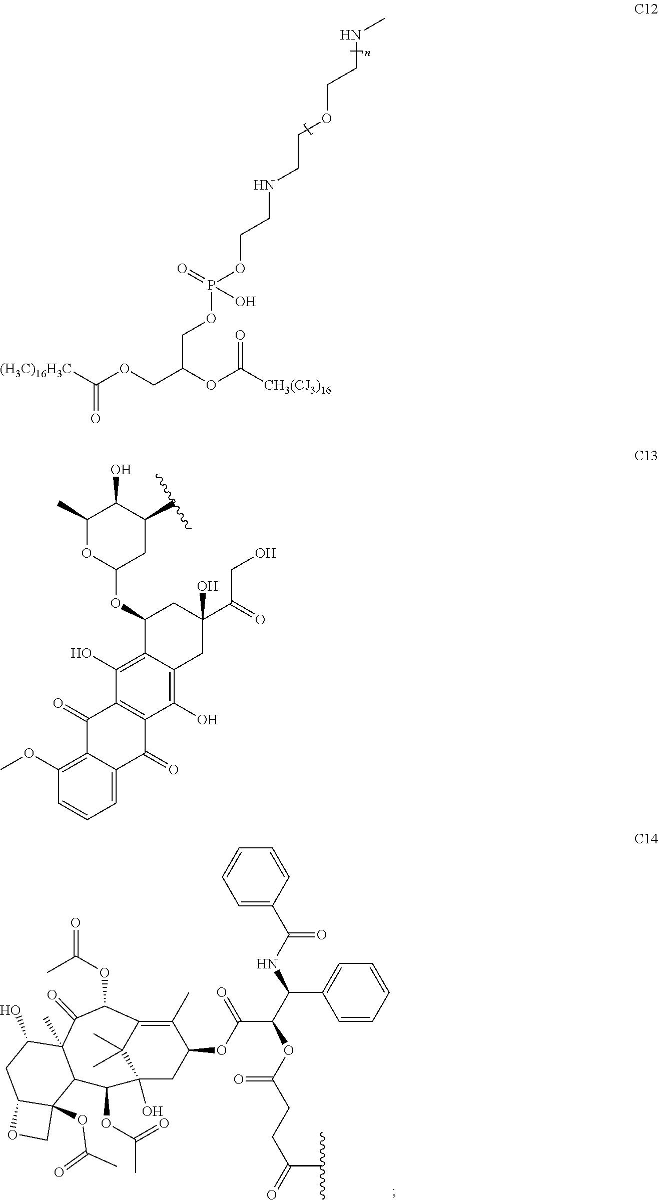
C also comprises C15, wherein the C15 is an amino acid chain with a sequence of SEQ ID NO: 1.

5. The vincamine derivative according to claim 1, wherein the vincamine derivative comprises any one of compound 1 to compound 38 having the following structural formulas: 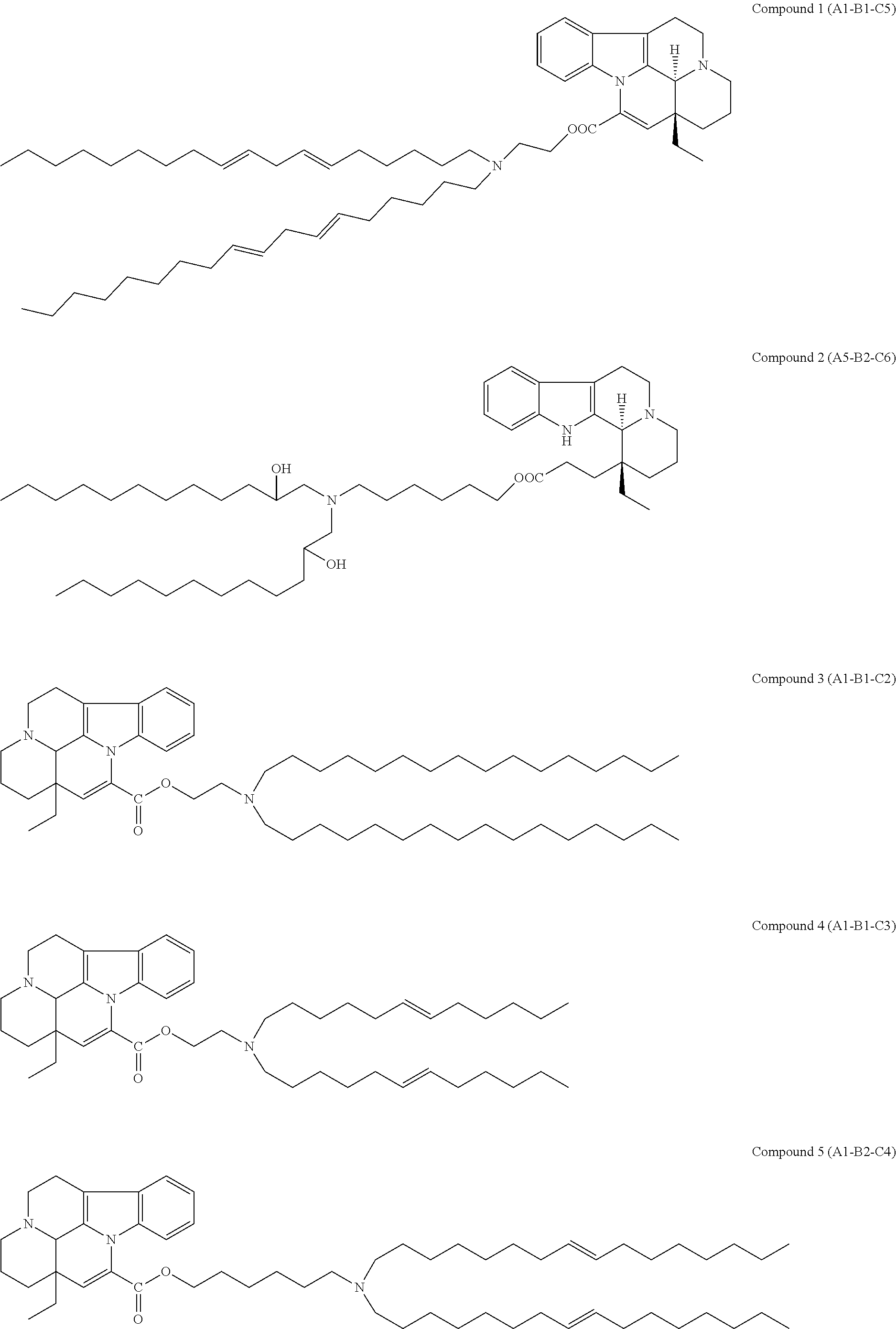 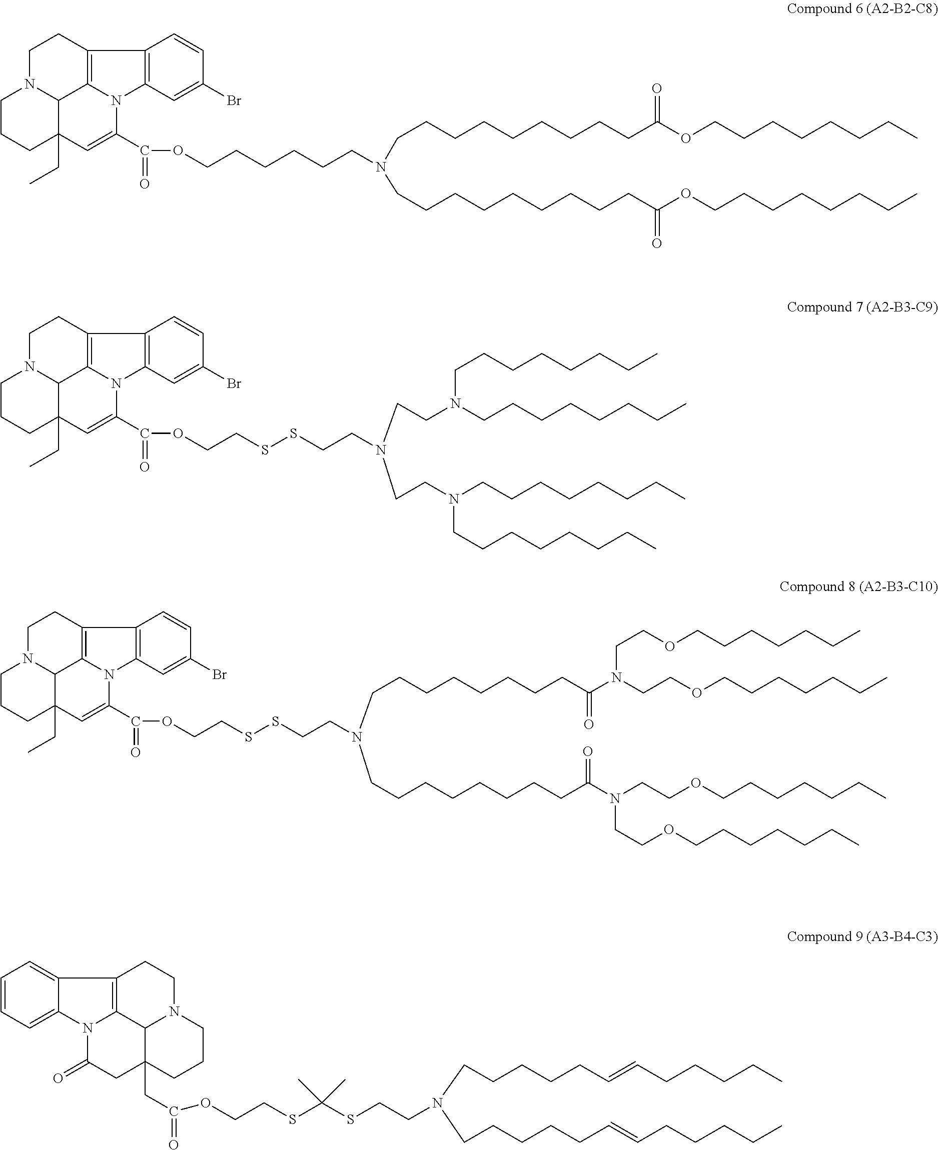 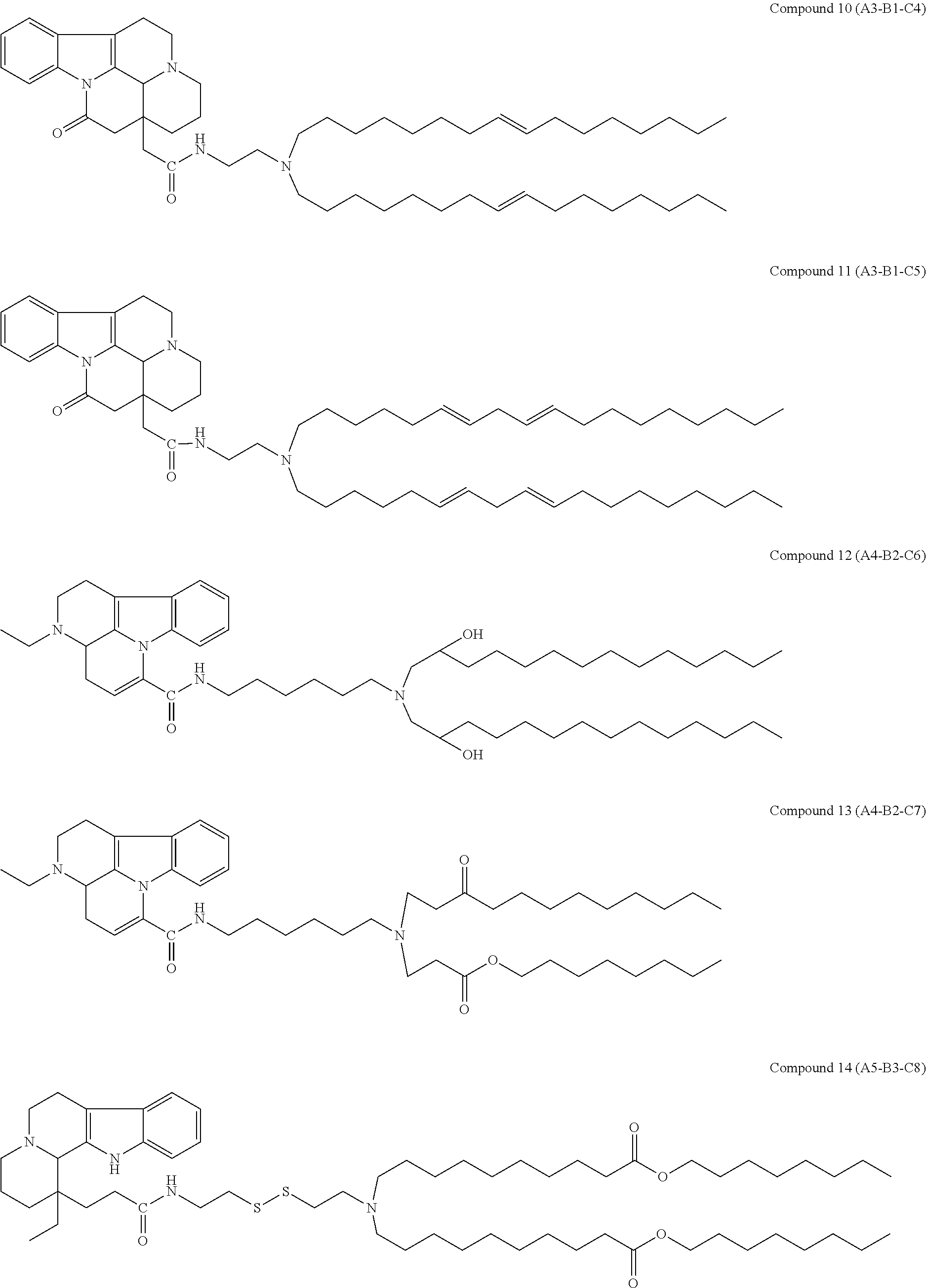 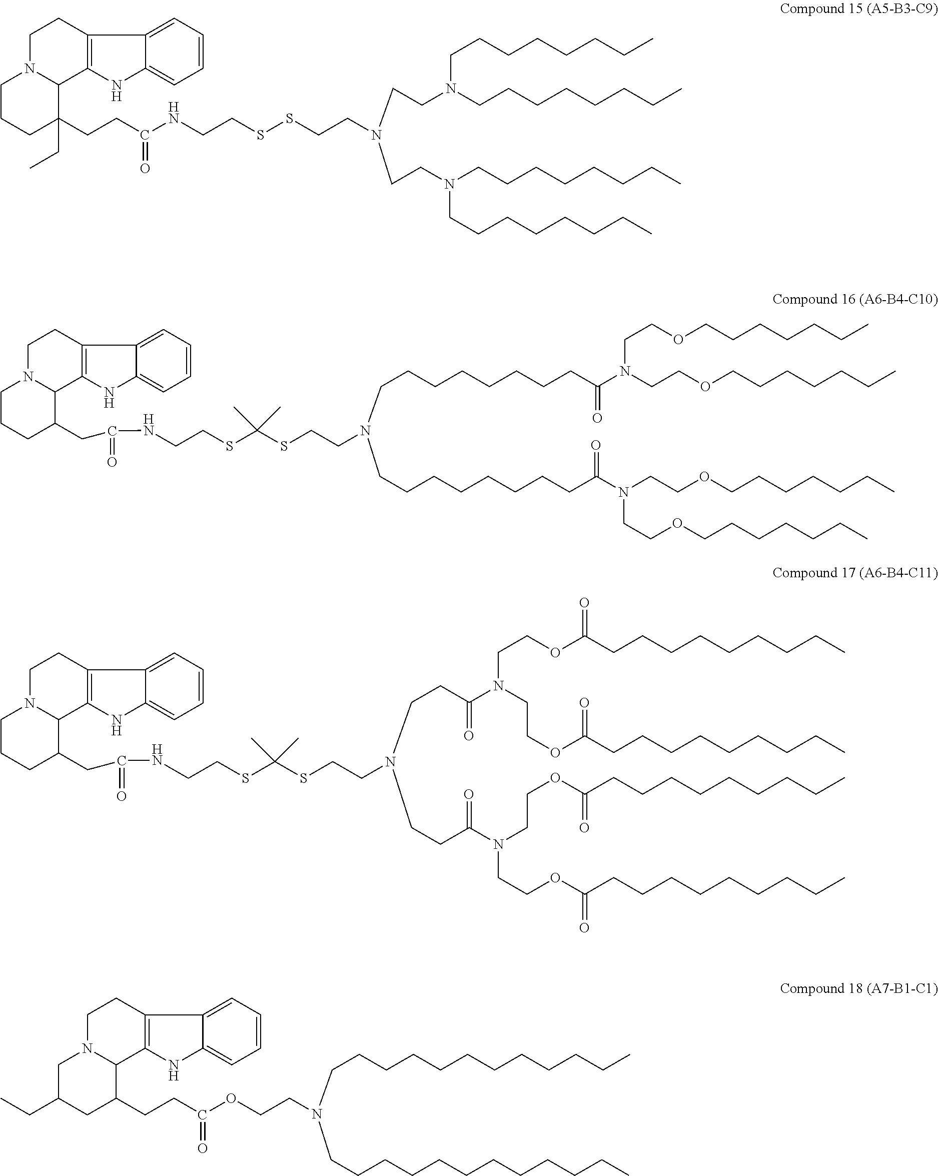 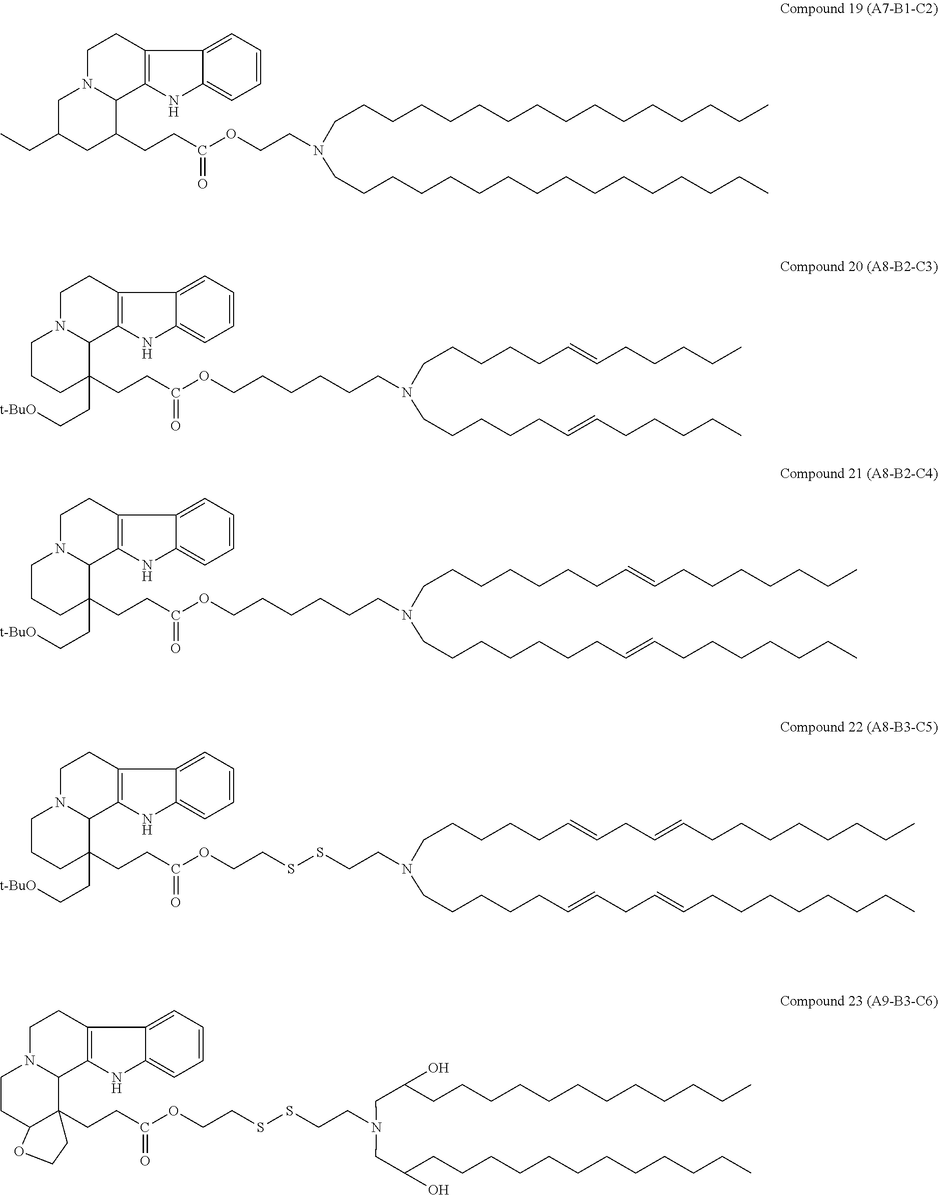 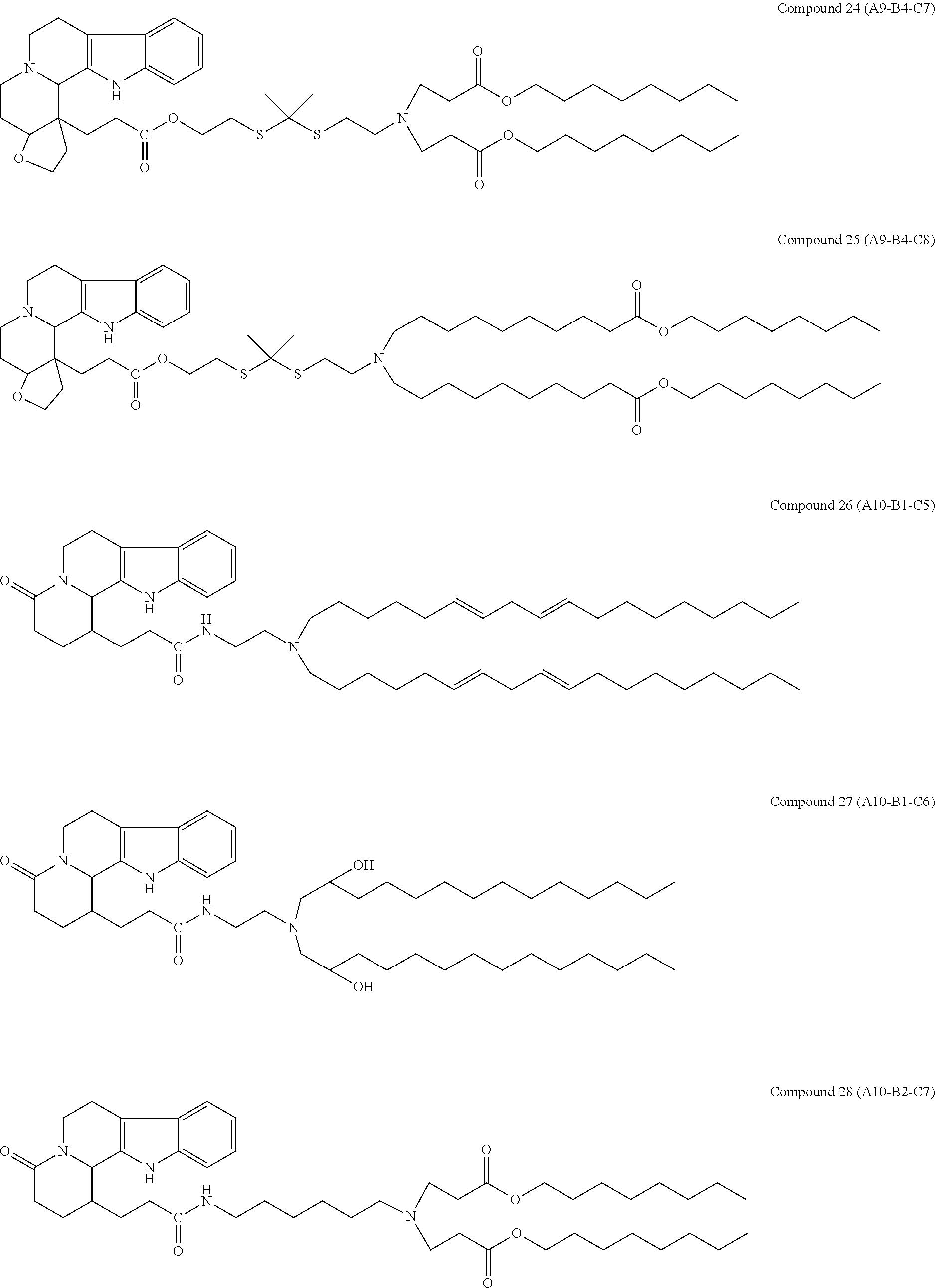 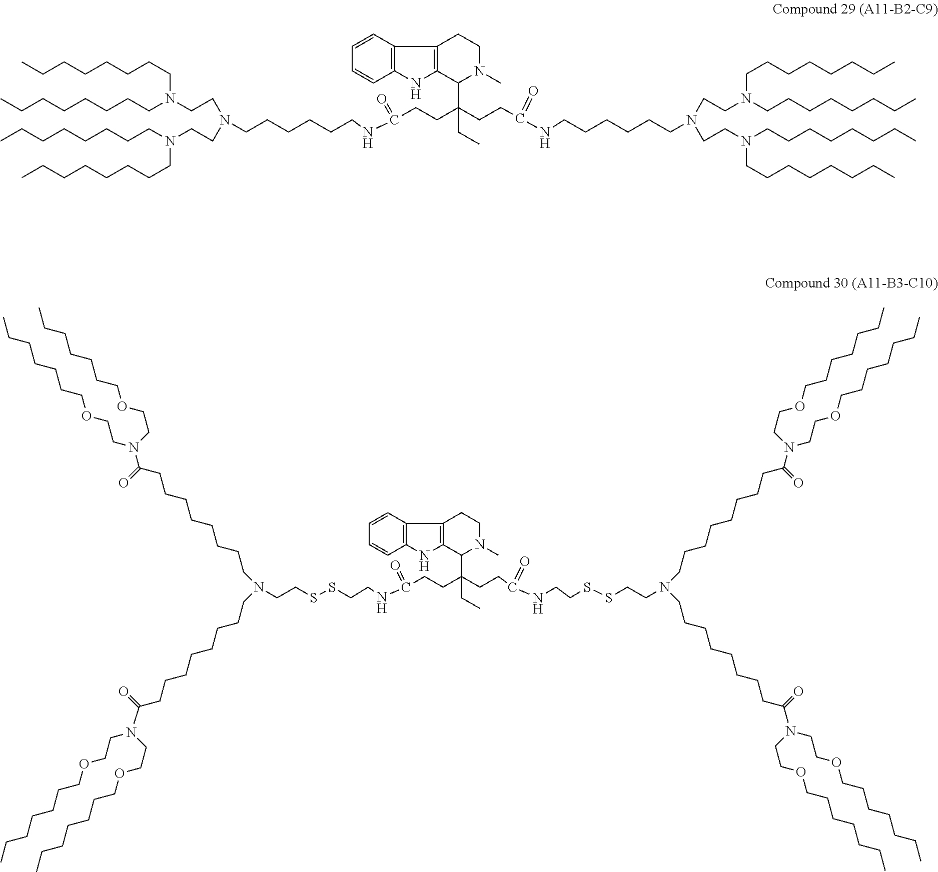 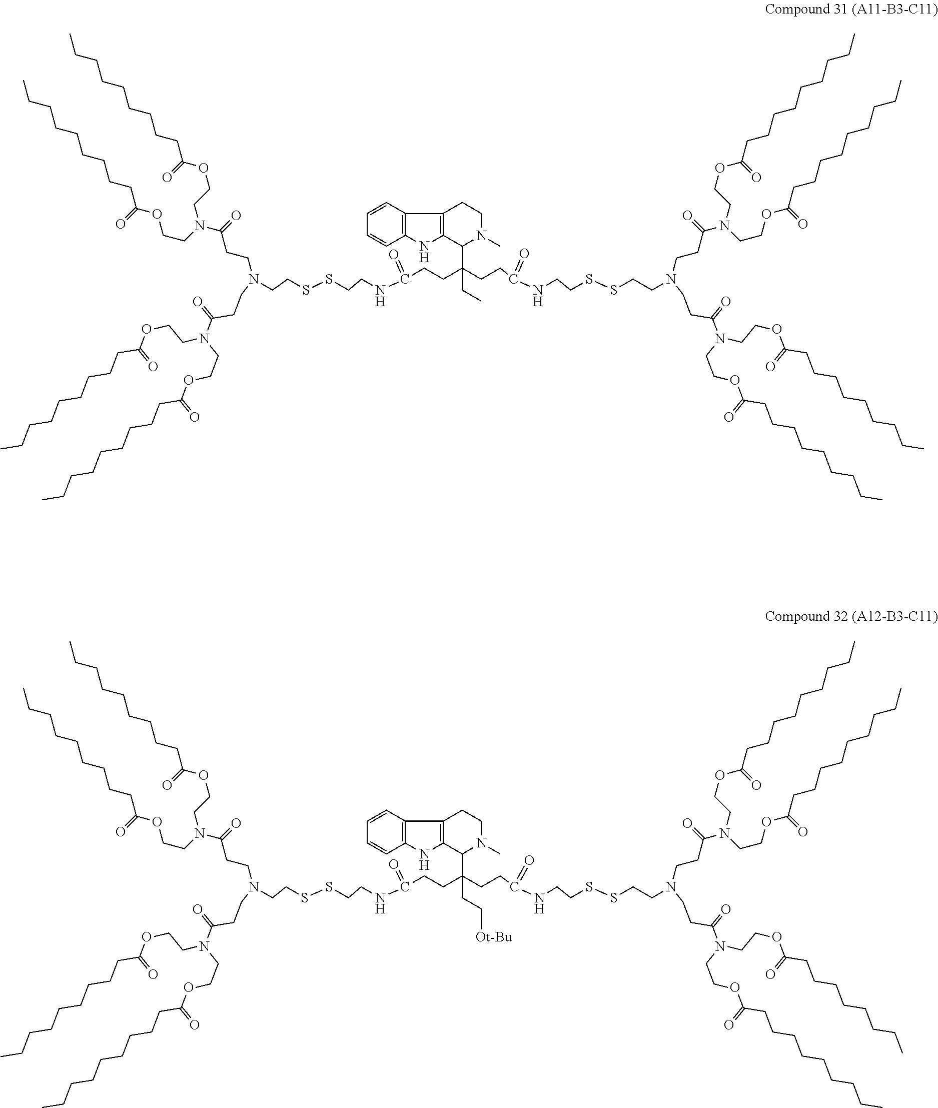 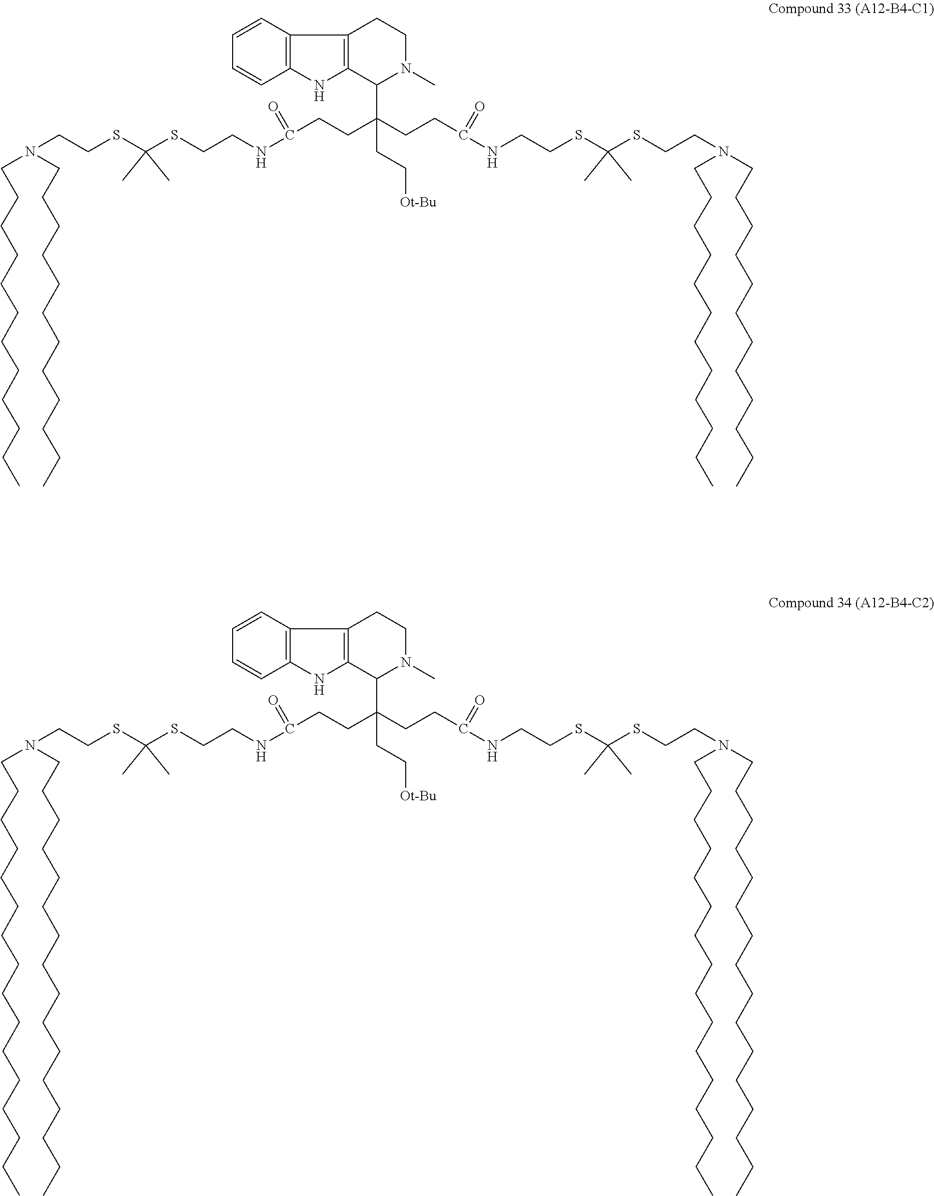 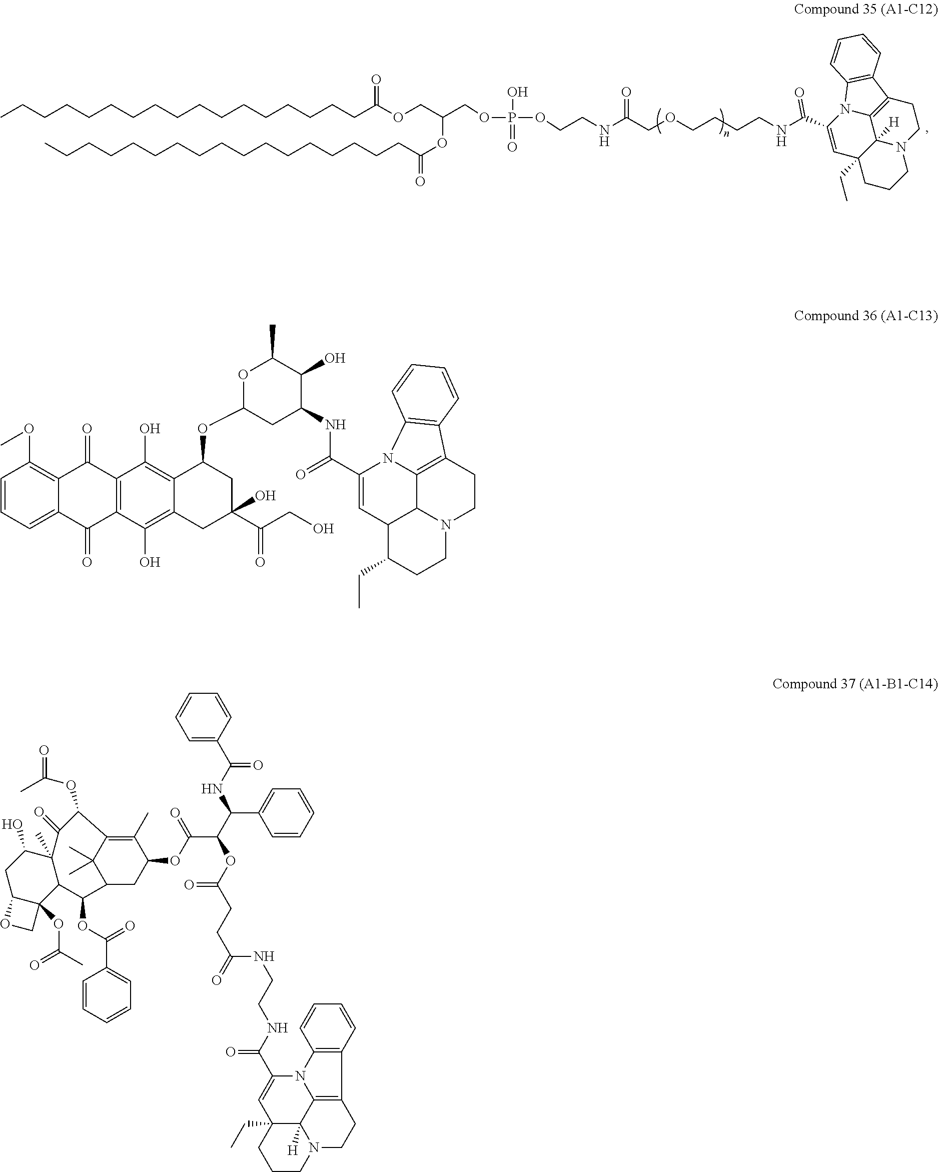 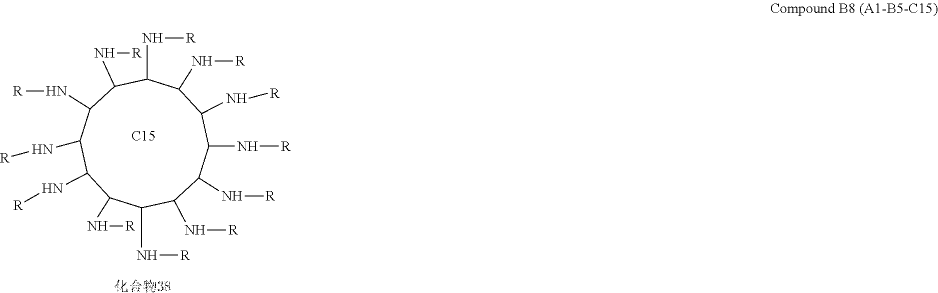
wherein C15 is an amino acid chain with a sequence of SEQ ID NO: 1, R is 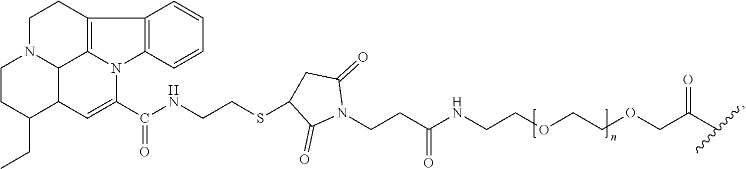
and n is a positive integer.

6. A preparation method of the vincamine derivative according to claim 1, wherein the preparation method comprises any one of Method I, Method II, Method III, and Method IV when the vincamine derivative has a structure represented by formula 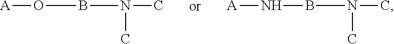
Method I comprises: when A is a group with the structural formula A1, B is a group with the structural formula B1, and C is a group with the structural formula C14, dissolving a compound having the structural formula A1-OH, a compound having the structural formula H₂N-B1-NH₂ and benzotriazol-1-yl-oxy-tris(pyrrolidino)phosphonium hexafluorophosphate in an organic solvent in a molar ratio of 0.17:0.27:0.28, stirring for 12-48 hours at 20-40° C., performing rotary evaporation, redissolution, extraction and drying on a product obtained by the reaction, concentrating under reduced pressure, and curing by using an organic solvent to obtain an intermediate compound I; dissolving a compound having the structural formula C14-OH, the intermediate compound I and benzotriazol-1-yl-oxy-tris(pyrrolidino)phosphonium hexafluorophosphate in an organic solvent in a molar ratio of 0.31:0.052:0.047, adjusting pH to 7-8, stirring and reacting at 20-40° C. for 1-12 hours, and performing rotary evaporation, redissolution, extraction and drying on a product obtained by the reaction, and concentrating under reduced pressure to obtain a vincamine derivative having the structural formula A1-NH-B1-NH-C14;
Method II comprises: when A is a group with the structural formula A1, B is a group of with the structural formula B5, and C is bovine serum albumin (BSA) having an amino acid sequence of SEQ ID NO: 1, dissolving a compound having the structural formula A1-OH, cystamine and benzotriazol-1-yl-oxy-tris(pyrrolidino)phosphonium hexafluorophosphate in an organic solvent in a molar ratio of 0.15:0.23:0.23, adjusting pH to 7-8 by using an organic solvent, stirring and reacting at 20-40° C. for 1-12 hours, curing a product obtained by the reaction with an organic solvent, drying in vacuum to obtain a crude product, dissolving the crude product in an organic solvent, dripping an aqueous solution of tris(2-carboxyethyl)phosphine hydrochloride (a molar ratio of the compound with the structural formula A1-OH to the tris(2-carboxyethyl)phosphine hydrochloride is 0.15:0.17), stirring and reacting at 20-40° C. for 1-12 hours, concentrating under reduced pressure, adding water, precipitating, and drying to obtain an intermediate compound II; dissolving the intermediate compound II and NHS-PEG₁-Mal in an organic solvent in a molar ratio of 0.02:0.01, stirring the reaction mixture at 20-40° C. for 1-12 hours, and adding the reaction mixture to a PBS buffer solution of the bovine serum albumin (BSA) having an amino acid sequence of SEQ ID NO: 1, stirring and reacting at 20-40° C. for 1-12 hours, dialyzing with pure water, and freeze-drying to obtain a vincamine derivative having the structural formula (A1-NH-B5-NH)₁₁-C15;
Method III: a preparation method of vincamine derivatives with the other structural formulas comprises the following steps:
(1) dissolving a compound having the structural formula C—Br, a compound having the structural formula HO—B—NH₂ or H₂N—B—NH₂, potassium carbonate and potassium iodide in an organic solvent in a molar ratio of 1.21:0.552:2.43:0.552, heating to 45-65° C., reacting for 12-48 hours, cooling and filtering a product obtained by the reaction, extracting an obtained filtrate with n-hexane, and separating by silica gel chromatography to obtain an intermediate compound having the structural formula 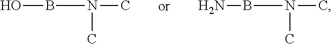
wherein B is any one of —(CH₂)ₙ—, —(CH₂)ₙ—S—S—(CH₂)ₙ—, or —(CH₂)ₙ-TK-(CH₂)ₙ—, and C is a group as defined above except for a hydroxyl-substituted linear alkyl group having 10 to 18 carbon atoms;
or dissolving an epoxy-substituted linear alkane group having 10 to 18 carbon atoms and a compound having the structural formula HO—B—NH₂ or H₂N—B—NH₂ in an organic solvent in a molar ratio of 1.26:0.57, heating to 55-75° C., reacting for 12-48 hours, concentrating a product obtained by the reaction under reduced pressure, and separating by silica gel chromatography to obtain an intermediate compound having the structural formula 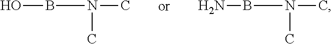
wherein C is a hydroxyl-substituted linear alkyl group having 10 to 18 carbon atoms;
(2) dissolving a compound having the structural formula A-OH, the intermediate compound having the structural formula 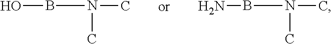
1-ethyl-3-(3-dimethylaminopropyl)carbodiimide hydrochloride and 4-dimethylaminopyridine in an organic solvent in a molar ratio of 0.15:1.59:0.183:0.0081, heating to 20-40° C., reacting for 24-96 hours, diluting and extracting a product obtained by the reaction, drying, concentrating under reduced pressure, and separating by silica gel chromatography to obtain a vincamine derivative having the structural formula 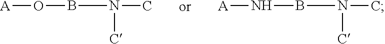
a preparation method of a vincamine derivative having the structural formula A-O—NH—C is as follows:
when A is a group with the structural formula A1 and C is a group with the structural formula C12, dissolving a compound having the structural formula A1-OH, a compound having the structural formula C12-NH₂(DSPE-PEG-NH₂) and benzotriazol-1-yl-oxy-tris(pyrrolidino)phosphonium hexafluorophosphate in an organic solvent, stirring and reacting at 20-40° C. for 24-96 hours, dialyzing with pure water, and freeze-drying to obtain a vincamine derivative having the structural formula A1-O—NH-C12; and
when A is a group with the structural formula A1 and C is a group with the structural formula C13, dissolving a compound having the structural formula A1-OH, a compound having the structural formula C13-NH₂(DOX-NH₂) and benzotriazol-1-yl-oxy-tris(pyrrolidino)phosphonium hexafluorophosphate in an organic solvent, adjusting pH to 7-8, stirring and reacting at 20-40° C. for 1-12 hours, and performing rotary evaporation, redissolution, extraction and drying on a product obtained by the reaction, concentrating under reduced pressure, and curing by using an organic solvent to obtain a vincamine derivative having the structural formula A1-O—NH-C13.

7. The preparation method according to claim 6, wherein an eluent used in the silica gel chromatography separation in the step (1) is a mixed solution of methanol and dichloromethane in a volume ratio of 10:90-90:10, and an eluent used in the silica gel chromatography separation in the step (2) is a mixed solution of acetone and n-hexane in a volume ratio of 20:80-80:20.

8. The preparation method according to claim 6, wherein the organic solvent is any one of acetonitrile, ethanol, methanol, N,N-dimethylformamide, N,N-diisopropylethylamine, ethyl acetate, methyl tert-butyl ether, triethylamine, pyridine, or dichloromethane.

9. Use of the vincamine derivative according to claim 1 in brain-targeted delivery of drugs for treating brain diseases.
